(19) United States
(12) Patent Application Publication
(10) Pub. No.: US 2024/0109850 A1
(43) Pub. Date: Apr. 4, 2024

(54) Covalent PPARG inverse-agonists

(71) Applicants: Bayer Aktiengesellschaft, Leverkusen (DE); The Broad Institute, Inc., Cambridge, MA (US); Dana-Farber Cancer Institute, Inc., Boston, MA (US)

(72) Inventors: Knut EIS, Newton, MA (US); Elisabeth POOK, Cambridge, MA (US); Ulf BRÜGGEMEIER, Leichlingen (DE); Adelaide Clara F. A. DE LEMOS, Somerville, MA (US); Sven CHRISTIAN, Somerville, MA (US); Isabel Sophie JERCHEL-FURAU, Concord, MA (US); Ulrike RAUH, Berlin (DE); Nico BRÄUER, Falkensee (DE); Timo STELLFELD, Berlin (DE); Anders Roland FRIBERG, Berlin (DE); Christian LECHNER, Berlin (DE); Stefan KAULFUSS, Berlin (DE); Hanna MEYER, Berlin (DE); Charlotte Christine KOPITZ, Falkensee (DE); Steven James FERRARA, Cambridge, MA (US); Jonathan GOLDSTEIN, Cambridge, MA (US); Matthew MEYERSON, Boston, MA (US); Christopher LEMKE, Cambridge, MA (US); Timothy A. LEWIS, Cambridge, MA (US)

(73) Assignees: Bayer Aktiengesellschaft, Leverkusen (DE); The Broad Institute, Inc., Cambridge, MA (US); Dana-Farber Cancer Institute, Inc., Boston, MA (US)

(21) Appl. No.: 18/241,147

(22) Filed: Aug. 31, 2023

Related U.S. Application Data
(60) Provisional application No. 63/403,846, filed on Sep. 5, 2022.

(51) Int. Cl. C07D 263/57; C07D 235/18; C07D 249/20; C07D 413/04; C07D 413/12

(52) U.S. Cl. CPC C07D 263/57; C07D 235/18; C07D 249/20; C07D 413/04; C07D 413/12

(57) ABSTRACT

The present invention includes compounds of formula (I)

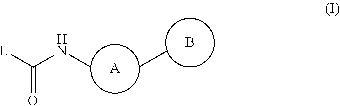

and methods for its preparation and treatment of disorders with activated peroxisome preoliferator-activated receptor gamma (PPARG), particularly cancer.

The present invention includes compositions and methods for treatment of disorders with activated peroxisome preoliferator-activated receptor gamma (PPARG). This invention more specifically includes novel covalent inverse agonists of PPARG that can be used to treat bladder cancer, pancreatic cancer, prostate cancer, colorectal cancer, esophagael cancer, gastric cancer, and breast cancer.

### CROSS-REFERENCE TO RELATED APPLICATION

This application claims priority to and the benefit U.S. Provisional Application No. 63/403,846, filed Sep. 5, 2022, the entire contents of which are incorporated by reference herein.

### BACKGROUND

Peroxisome Proliferator Activated Receptor Gamma (PPARG) is a ligand-activated transcription factor that regulates the expression of target genes. PPARG requires heterodimerization with a member of its obligate nuclear receptor partner retinoid X receptor (RXR); RXR-alpha (RXRA), RXR-beta (RXRB), or RXR-gamma (RXRG). Together they bind to DNA response elements in the promoter and enhancer regions of target genes. The PPARG/RXR complex interacts with nuclear receptor coactivators (NCOA1 and others) and nuclear receptor co-repressors NCOR1 and NCOR2, which recruit additional transcriptional regulatory proteins including histone acetyl-transferases and histone deacetylases, mediator complex (MED1) as well as the basal transcriptional machinery. These dynamic interactions lead to regukation of target gene expression.

Many canonical target genes of the PPARG transcription complex are involved in the regulation of lipid and glucose homeostasis. PPARG agonists improve glucose tolerance and insulin sensitivity, and the thiazolidinedione (glitazone) and glitazar anti-diabetic drug classes exploit this pharmacology for their therapeutic effect. The anti-diabetic thiazolidinedione drugs including rosiglitazone and pioglitazone are selective PPARG agonists, while members of the glitazar family (including saroglitazar and other failed drug candidates) are dual agonists of both PPARA and PPARG. Interestingly, high doses of the PPARG agonist pioglitazone is associated with an increased risk of bladder cancer in rodents and patients, which limited clinical use. Additionally, high incidence of bladder cancer in rodent toxicity studies during pre-clinical development of glitazars has been a challenge for clinical development of this class.

Peroxisome proliferator-activated receptor gamma (PPARG) activated cancers (e.g., breast cancer, esophageal cancer, pancreatic cancer, colorectal cancer, hepatocellular cancer, bladder cancer, and the like) represent a significant class of cancers. For example, bladder cancer is the fifth most commonly diagnosed cancer type in the United States. About half of all bladder cancer patients are diagnosed with non-invasive/superficial urothelial carcinoma of the bladder and respond well to existing chemotherapy regimens, with a 5-year survival rate of 96%. Patients diagnosed with invasive disease have a poorer prognosis, with a 5-year survival rate of 70% or less, depending upon extent of invasion beyond the bladder. Currently, there are no FDA-approved targeted therapeutics available to these patients. While recent approvals of immune checkpoint inhibitors (ICI) have helped prognosis in a subset of patients, there is still a need for additional therapeutoc options and potential therapeutiocs to enhance the activity of ICIs.

PPARG is an enriched genetic dependency in luminal bladder cancer. Hotspot mutations of the heterodimeric partner RXRA at S427 and focal gene ampli-fication of PPARG lead to ligand-independent activation of PPARG and appear to be oncogenic through multiple mechanisms. PPARG inverse agonists exemplified by T0070907 and SR10221 which inhibit gene transactivation by promoting a repressive conformation and show selective anti-proliferative activity in vitro against PPARG-dependent cell lines. Inverse agonists would have the opposite effect of an agonist and counteract the effects of PPARG activation in cancers and other conditions including metabolism, bone biology, and inflammation.

PPARG is in the top ten most enriched genetic dependencies in pancreatic cancer and cancer of the urinary tract lineage within the Cancer Dependency Map genome-wide CRISPR loss-of-function screen across a panel of over 1000 cell lines (DepMap.org). Pancreatic cancer and cancer of the urinary tract lineage have amongst the highest average gene expression profiles for PPARG amongst all lineages indicating a potential patient population for consideration. There are very limited therapeutic options for patients with pancreatic cancer and prognosis remains grim.

Accordingly, there is an urgent need for compositions and methods for treating PPARG activated cancers such as bladder and pancreatic cancer.

### SUMMARY

The present disclosure relates, at least in part, to the discovery of peroxisome proliferator-activated receptor gamma (PPARG) inverse agonists as potential therapeutic for the treatment of various PPARG activated cancers such as, for example, breast cancer, bladder cancer, colorectal cancer, esophageal cancer, gastric cancer, hepatocellular cancer, pancreatic cancer, and prostate cancer. As described herein, the present disclosure provides PPARG modulators, more specifically inverse-agonists, that are able to down regulate PPARG signaling in PPARG activated cancers, thereby decreasing cellular proliferation associated with PPARG activated cancers. In particular, the present disclosure provides inverse-agonists that are able to reverse up-regulation of PPARG signaling in PPARG activated cancers, thereby providing a therapeutic modality capable of treating PPARG activated cancers such as, for example, cancer of the urinary tract, particularly bladder cancer or pancreatic cancer.

In an aspect, the present disclosure provides a method of treating a subject having a peroxisome proliferator-activated receptor gamma (PPARG) activated cancer that includes a step of administering a therapeutically effective amount of a PPARG signaling modulator, particularly an inverse-agonist to the subject.

In an embodiment, the PPARG inverse agonists are a compounds of formula (I)
1. A compound of formula (I)

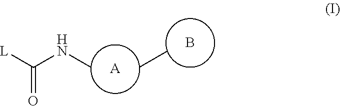

wherein,
L is selected from

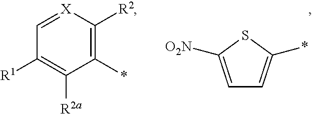

and an ethenylene group
R¹ is selected from a hydrogen atom, a NO₂ group, a cyano group, a —C═(O)NH—(C₁-C₃-alkyl) group, and C₁-C₃-haloalkyl group;
R² is selected from a hydrogen atom and a halogen atom;
R^{2a} is a hydrogen atom or a halogen atom;
X is a CR³ group and a nitrogen atom
R³ is selected from the group consisting of a hydrogen atom or a halogen atom;
Ring A is selected from the groups (a) to (h)

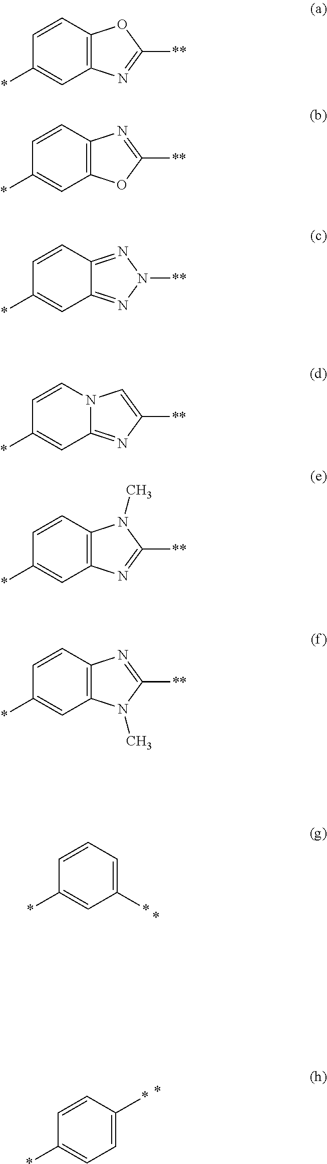

whereby * marks the position where ring A is bound to the nitrogen atom of the amide and ** marks the position where ring A is bound to ring B;
B is selected from the groups

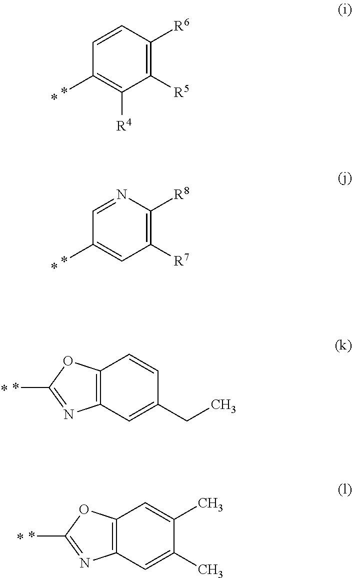

whereby ** is the point of attachment to the A ring,
R⁴ is selected from the group consisting of a hydrogen atom, a halogen atom and a C1-C₃-alkyl group;
R⁵ is selected from the group consisting of a hydrogen atom, a halogen atom, a C1-C₃-alkyl group;
R⁶ is selected from the group consisting of a hydrogen atom, a C₁-C₃-alkyl group, a C1-C₃-alkoxy group;
R⁷ is selected from a hydrogen atom or a C₁-C₃-alkyl group;
R⁸ is selected from a hydrogen atom or a C₁-C₃-alkyl group;
   with the proviso, that those compounds are excluded for which
   R¹ is a nitro group,
   R² is a chlorine atom,
   R^{2a} is a hydrogen atom,
   X is a CH group,
   ring A is a benzoxazole (a),
   R⁴ is selected from the group consisting of a hydrogen atom, a halogen atom and a
   C₁-C₃-alkyl group,
   R⁵ is selected from the group consisting of a hydrogen atom, a halogen atom, a C₁-C₃-alkyl group and
   R⁶ is selected from the group consisting of a hydrogen atom, a C₁-C₃-alkyl group, a C₁-C₃-alkoxy group,
or a stereoisomer, a tautomer, an N-oxide, a hydrate, a solvate, or a salt thereof, or a mixture of same and combinations with other anti-cancer agents

In an embodiment, the PPARG signaling modulator is an inverse-agonist of PPARG signaling.

### BRIEF DESCRIPTION OF THE DRAWINGS

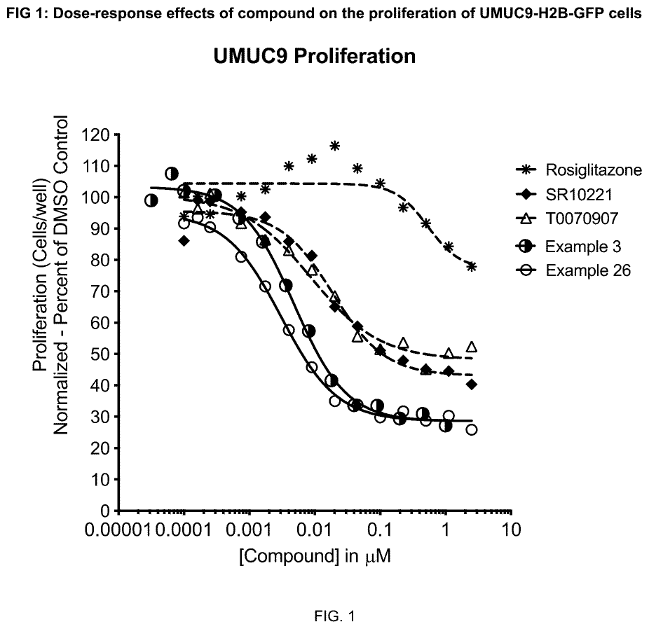

FIG. 1 (FIG. 1): shows the dose-response effects of the compound of example 3 on the proliferation of UMUC9-H2B-GFP bladder cancer cells.
Open box □: Rosiglitazone
Closed triangle ▴: SR10221
Open Triangle Δ: T007907
Open circle ∘: compound of example 26
Closed circle •: compound of example 3
SR10221, is an inverse PPARG agonist, having the structure:

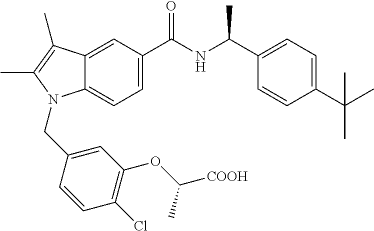 [SMILES: Cc1c(C)n(Cc2ccc(Cl)c(O[C@@H](C)C(=O)O)c2)c2ccc(C(=O)N[C@@H](C)c3ccc(C(C)(C)C)cc3)cc12]

(Nature communications, Jun. 12, 2015, DOI:10.1038/ncomms8443)
and T0070907 is a PPARG antagonist, CAS No.: 313516-66-4, having the structure:

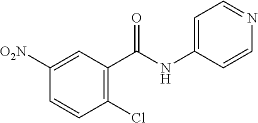 [SMILES: O=C(Nc1ccncc1)c1cc([N+](=O)[O-])ccc1Cl]

and
Rosiglitazone is a marketed antidiabetic drug binding PPARG having the structure:

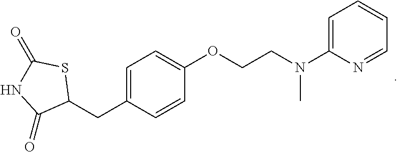 [SMILES: CN(CCOc1ccc(CC2SC(=O)NC2=O)cc1)c1ccccn1]

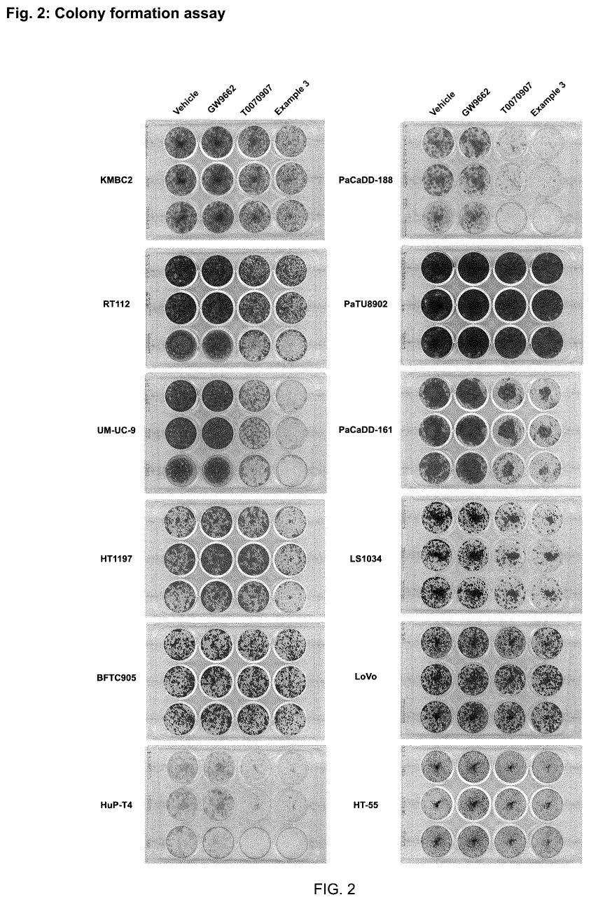

FIG. 2 (FIG. 2): shows the result of a colony formation assay performed with the compound of example 3 and several cancer cell lines:
Bladder cancer cell lines: KMBC2, RT112, UM-UC-9, HT1197, BFTC905
Pancreatic cancer cell lines: Hup-T4, PaCaDD-188, PaTU8902, PaCaDD-161
and
Colorectal cancer cell cancer lines: LS1034, LoVo, HT-55.
GW9662 is a commercially available PPARG antagonist, CAS. Nr.: 22978-25-2, having the structure:

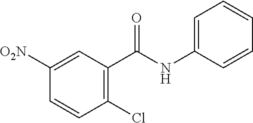 [SMILES: O=C(Nc1ccccc1)c1cc([N+](=O)[O-])ccc1Cl]

and T0070907 is another PPARG antagonist, CAS No.: 313516-66-4, having the structure:

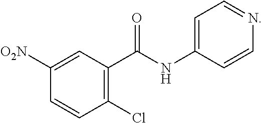 [SMILES: O=C(Nc1ccncc1)c1cc([N+](=O)[O-])ccc1Cl]

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

Structures drawn include all permissible rotations about bonds.

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen atom.

Commonly, it is possible for the number of optional substituents, when present, to be 1, 2, 3, in particular 1, or 2.

As used herein, the term “one or more”, e.g. in the definition of the substituents of the compounds of general formula (I) of the present invention, means “1, 2, 3, 4 or 5, particularly 1, 2, 3 or 4, more particularly 1, 2 or 3, even more particularly 1 or 2”.

As used herein, an oxo substituent represents an oxygen atom, which is bound to a carbon atom or to a sulfur atom via a double bond.

The term “ring substituent” means a substituent attached to an aromatic or nonaromatic ring which replaces an available hydrogen atom on the ring.

Should a composite substituent be composed of more than one parts, e.g. (C₁-C₄-alkyl)-O—(C₁-C₄-alkyl)-, a hyphen at the beginning or at the end of such a composite substituent indicates the point of attachment of said composite substituent to the rest of the molecule. Should the composite substituent be substituted said substitutent may be bound at any suitable carbon atom of the composite substitutent.

Should a ring, comprising carbon atoms and optionally one or more heteroatoms, such as nitrogen, oxygen or sulfur atoms for example, be substituted with a substituent, it is possible for said substituent to be bound at any suitable position of said ring, be it bound to a suitable carbon atom and/or to a suitable heteroatom.

The term “comprising” when used in the specification includes “consisting of”.

If within the present text any item is referred to as “as mentioned herein”, it means that it may be mentioned anywhere in the present text.

The terms as mentioned in the present text have the following meanings:

The term “halogen atom” means a fluorine, chlorine, bromine or iodine atom, particularly a fluorine, chlorine or bromine atom.

The term “C₁-C₆-alkyl” means a linear or branched, saturated, monovalent hydrocarbon group having 1, 2, 3, 4, 5 or 6 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl, 1,1-dimethylpropyl, hexyl, 1-methylpentyl, 2-methylpentyl, 3-methylpentyl, 4-methylpentyl, 1-ethylbutyl, 2-ethylbutyl, 1,1-dimethylbutyl, 2,2-dimethylbutyl, 3,3-dimethylbutyl, 2,3-dimethylbutyl, 1,2-dimethylbutyl or 1,3-dimethylbutyl group, or an isomer thereof. Particularly, said group has 1, 2, 3 or 4 carbon atoms (“C₁-C₄-alkyl”), e.g. a methyl, ethyl, propyl, isopropyl, butyl, sec-butyl isobutyl, or tert-butyl group, more particularly 1, 2 or 3 carbon atoms (“C₁-C₃-alkyl”), e.g. a methyl, ethyl, n-propyl or isopropyl group.

The term “alkenylene” derives from the term “alkenyl” as being a bivalent constituent bearing a double bond and being named by addition of “ene” to the term “alkenyl” e.g. “ethenyl” becomes “ethenylene” meaning a “—CH═CH—” constituent whereby the open bonds of branched constituents are located at the respective ends of the longest chain.

The term “C₁-C₆-alkoxy” means a linear or branched, saturated, monovalent group of formula (C₁-C₆-alkyl)-O—, in which the term “C₁-C₆-alkyl” is as defined supra, e.g. a methoxy, ethoxy, n-propoxy, isopropoxy, n-butoxy, sec-butoxy, isobutoxy, tert-butoxy, pentyloxy, isopentyloxy or n-hexyloxy group, or an isomer thereof.

The term “heteroaryl” means a monovalent, monocyclic or bicyclic aromatic ring having 5, to 9 ring atoms (a “5- to 9-membered heteroaryl” group), containing at least one ring heteroatom and optionally one, two or three further ring heteroatoms from the series: N, O and/or S, and which is bound via a ring carbon atom to the rest of the molecule.

Said heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl, thiadiazolyl or tetrazolyl; or a 6-membered heteroaryl group, such as, for example, pyridinyl, pyridazinyl, pyrimidinyl, pyrazinyl or triazinyl; or bicyclic heteroaryl groups such as e.g. benzoxazole, benzimidazole, bentotriazole, imidazo[1,2-a]pyridine or a tricyclic heteroaryl group, such as, for example, carbazolyl, acridinyl or phenazinyl.

In general, and unless otherwise mentioned, the heteroaryl or heteroarylene groups include all possible isomeric forms thereof, e.g.: tautomers and positional isomers with respect to the point of linkage to the rest of the molecule. Thus, for some illustrative non-restricting examples, the term pyridinyl includes pyridin-2-yl, pyridin-3-yl and pyridin-4-yl; or the term thienyl includes thien-2-yl and thien-3-yl.

The term “C₁-C₆”, as used in the present text, e.g. in the context of the definition of “C₁-C₆-alkyl”, “C₁-C₆-haloalkyl”, “C₁-C₆-alkoxy” or “C₁-C₆-haloalkoxy” means an alkyl group having a finite number of carbon atoms of 1 to 6, i.e. 1, 2, 3, 4, 5 or 6 carbon atoms.

Further, as used herein, the term “C₃-C₈”, as used in the present text, e.g. in the context of the definition of “C₃-C₈-cycloalkyl”, means a cycloalkyl group having a finite number of carbon atoms of 3 to 8, i.e. 3, 4, 5, 6, 7 or 8 carbon atoms.

When a range of values is given, said range encompasses each value and sub-range within said range.

For example:
“C₁-C₆” encompasses C₁, C₂, C₃, C₄, C₅, C₆, C₁-C₆, C₁-C₅, C₁-C₄, C₁-C₃, C₁-C₂, C₂- C₆, C₂-C₅, C₂-C₄, C₂-C₃, C₃-C₆, C₃-C₅, C₃-C₄, C₄-C₆, C₄-C₅, and C₅-C₆;
“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;
“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;
“C₃-C₆” encompasses C₃, C₄, C₅, C₆, C₃-C₆, C₃-C₅, C₃-C₄, C₄-C₆, C₄-C₅, and C₅-C₆;
“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;
“C₄-C₇” encompasses C₄, C₅, C₆, C₇, C₄-C₇, C₄-C₆, C₄-C₅, C₅-C₇, C₅-C₆ and C₆-C₇;
“C₄-C₆” encompasses C₄, C₅, C₆, C₄-C₆, C₄-C₅ and C₅-C₆;
“C₅-C₁₀” encompasses C₅, C₆, C₇, C₈, C₉, C₁₀, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;
“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

By “agent” is meant any small molecule chemical compound, antibody, nucleic acid molecule, or polypeptide, or fragments thereof.

“Alkylating agents” directly damage DNA to prevent the cancer cell from reproducing. As a class of drugs, these agents are not phase-specific; in other words, they work in all phases of the cell cycle. Alkylating agents are used to treat many different cancers. Examples of alkylating agents include, for example, nitrogen mustards (e.g., mechlorethamine, chlorambucil, cyclophosphamide (Cytoxan®), ifosfamide, and melphalan), alkyl sulfonates (e.g., busulfan), triazines (e.g., dacarbazine (DTIC), temozolomide (Temodar®)), Nitrosoureas (including streptozocin, carmustine (BCNU), and Iomustine), and ethylenimines (e.g., thiotepa and altretamine). In addition, platinum drugs (e.g., cisplatin, carboplatin, and oxalaplatin) are often considered alkylating agents because they kill cancer cells in a similar way. The disclosure contemplates all of these drugs, or combinations thereof.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

“Anti-metabolites” are a class of drugs that interfere with DNA and RNA growth by substituting for the normal building blocks of RNA and DNA. These agents damage cells during the S phase. They are commonly used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), Capecitabine (Xeloda®), Cladribine, Clofarabine, Cytarabine (Ara-C®), Floxuridine, Fludarabine, Gemcitabine (Gemzar®), Hydroxyurea, Methotrexate, Pemetrexed (Alimta®), Pentostatin, and Thioguanine.

“Anti-microtubule agents” interfere with the microtubule function, some of them directly binding to the soluble tubulin or to the tubulin in the mircrotubules. Microtubules are building the spindle apparatus during mitosis. Examples for these agents are the taxanes, like docetaxel, paclitaxel, and the vinca alkaloids like vincristine, vinblastine, vindesine.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “disease” is meant any condition or disease that damages or interferes with the normal function of a cell, tissue, or organ.

By “effective amount” is meant the amount of a compound described herein required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount In still other embodiments, the PDE3A modulator is a compound of formula (I).

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)sulfonyl]oxy, (phenylsulfonyl)oxy, [(4-methylphenyl)sulfonyl]oxy, [(4-bromophenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethylphenyl)sulfonyl]oxy, [(4-tert-butylphenyl)sulfonyl]oxy and [(4-methoxyphenyl)sulfonyl]oxy.

Unless specifically stated or obvious from context, as used herein, the term “or” is understood to be inclusive. Unless specifically stated or obvious from context, as used herein, the terms “a”, “an”, and “the” are understood to be singular or plural.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

PPARG modulators are compounds that bind to PPARG and can generally alter its function. Modulators can further be subdivided into agonists, antagonists, and inverse agonists. An agonist will lead to the canonical activation of PPARG resulting in the upregulation of canonical target genes. An inverse-agonist will have the opposite effect of an agonist, and lead to the repression of canonical target genes. While an antagonist can be neutral on its own, yet block the binding and activity of either agonist or antagonist.

The term “prodrugs” or “prodrug” designates compounds which themselves can be biologically active or inactive, but are converted (for example metabolically or hydrolytically) into compounds according to the invention during their residence time in the body. Derivatives of the compound 6 and the salts thereof which are converted into compound 6 or a salt thereof in a biological system (bioprecursors or pro-drugs) are covered by the invention. Said biological system may be, for example, a mammalian organism, particularly a human subject. The bioprecursor is, for example, converted into a compound of formula (I) or a salt thereof by metabolic processes.

Unless specifically stated or obvious from context, as used herein, if a range is provided, the upper and lower limit are always meant to be included. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reference” is meant a standard or control condition.

“Topoisomerase inhibitors” interfere with enzymes called topoisomerases, which help separate the strands of DNA so they can be copied. They are used to treat certain leukemias, as well as lung-, ovarian-, gastrointestinal-, and other cancers. Examples of topoisomerase 1 inhibitors include topotecan and irinotecan (CPT-11). Examples of topoisomerase II inhibitors include etoposide (VP-16), teniposide and Mitoxantrone.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

### DETAILED DESCRIPTION

As a first aspect the invention provides compounds of formula (I)

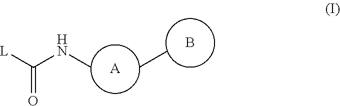

wherein,
L is selected from

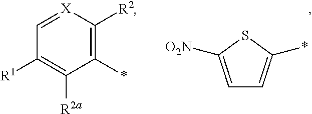

and an ethenylene group
R¹ is selected from a hydrogen atom, a NO₂ group, a cyano group, a —C═(O)NH—(C₁-C₃-alkyl) group, and C₁-C₃-haloalkyl group;
R² is selected from a hydrogen atom and a halogen atom;
R^{2a} is a hydrogen atom or a halogen atom;
X is a CR³ group and a nitrogen atom
R³ is selected from the group consisting of a hydrogen atom or a halogen atom;
Ring A is selected from the groups (a) to (h)

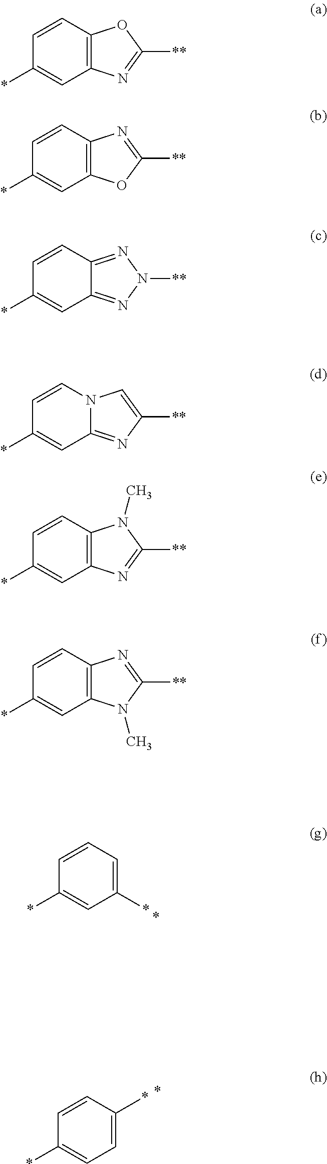

whereby * marks the position where ring A is bound to the nitrogen atom of the amide and ** marks the position where ring A is bound to ring B;
B is selected from the groups

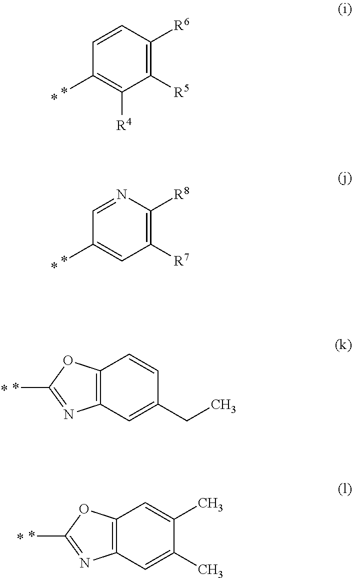

whereby ** is the point of attachment to the A ring,
R⁴ is selected from the group consisting of a hydrogen atom, a halogen atom and a C₁-C₃-alkyl group;
R⁵ is selected from the group consisting of a hydrogen atom, a halogen atom, a C₁-C₃-alkyl group;
R⁶ is selected from the group consisting of a hydrogen atom, a C₁-C₃-alkyl group, and a C₁-C₃-alkoxy group,
R⁷ is selected from the group consisting of a hydrogen atom and a C₁-C₃-alkyl group;
R⁸ is selected from the group consisting of a hydrogen atom and a C₁-C₃-alkyl group;
   with the proviso, that those compounds are excluded for which
   R¹ is a nitro group,
   R² is a chlorine atom,
   R^{2a} is a hydrogen atom,
   X is a CH group,
   ring A is a benzoxazole (a),
   R⁴ is selected from the group consisting of a hydrogen atom, a halogen atom and a
   C₁-C₃-alkyl group,
   R⁵ is selected from the group consisting of a hydrogen atom, a halogen atom, a C₁-C₃-alkyl group and
   R⁶ is selected from the group consisting of a hydrogen atom, a C₁-C₃-alkyl group, a C₁-C₃-alkoxy group,
or a stereoisomer, a tautomer, an N-oxide, a hydrate, a solvate, or a salt thereof, or a mixture of same as well as combinations thereof and methods for their preparation.

The following compounds of this class are known and hence are disclaimed from claim 1 by the proviso:

| No | name |
|---|---|
| 1 | 2-Chloro-5-nitro-N-(2-phenyl-5-benzoxazolyl)benzamide, |
| 2 | 2-Chloro-N-[2-(2-chlorophenyl)-5-benzoxazolyl]-5-nitrobenzamide, |
| 3 | N-[2-(2-Bromophenyl)-5-benzoxazolyl]-2-chloro-5-nitrobenzamide, |
| 4 | 2-Chloro-N-[2-(3,4-dimethoxyphenyl)-5-benzoxazolyl]-5-nitrobenzamide, |
| 5 | 2-Chloro-N-[2-(4-fluorophenyl)-5-benzoxazolyl]-5-nitroenzamide, |
| 6 | 2-Chloro-N-[2-(2-fluorophenyl)-5-benzoxazolyl]-5-nitrobenzamide, |
| 7 | 2-Chloro-N-[2-(2-methylphenyl)-5-benzoxazolyl]5-nitrobenzamide, |
| 8 | 2-Chloro-5-nitro-N-[2-(3,4,5-trimethoxyphenyl)-5-benzoxazolyl]benzamide, |
| 9 | 2-Chloro-N-[2-(3-methylphenyl)-5-benzoxazolyl]-5-nitrobenzamide, |
| 10 | 2-Chloro-N-[2-(4-ethylphenyl)-5-benzoxazolyl]-5-nitrobenzamide, |
| 11 | 2-Chloro-N-[2-(3-iodohenyl)-5-benzoxyzolyl]-5-nitrobenzamide, |
| 12 | 2-Chloro-N-[2-(3,5-dimethyphenyl)5-benzoxazolyl]-5-nitrobenzamide, |
| 13 | 2-Chloro-N-[2-(4-iodophenyl)-5-benzoxazolyl]-5-nitrobenzamide, |
| 14 | 2-Chloro-N-[2-(4-chlorophenyl)-5-benzoxazolyl]-5-nitrobenzamide, |
| 15 | N-[2-(4-Bromophenyl)-5-benzoxazolyl]-2-chloro-5-nitrobenzamide, |
| 16 | 2-Chloro-N-[2-(3,4-dimethylphenyl-5-benzoxazolyl]-5-nitrobenzamide, |
| 17 | N-[2-(3-Bromophenyl)-5-benzoxazolyl]-2-chloro-5-nitrobenzamide, |
| 18 | 2-Chloro-N-[2-(3-chlorophenl)-5-benzoxazolyl]-5-nitrobenzamide, |
| 19 | 2-Chloro-N-[2-(1-naphthalenyl)-5-benzoxazolyl]-5-nitrobenzamide, |
| 20 | N-(2-[1,1′-Biphenyl]-4-yl-5-benzoxazolyl)-2-chloro-5-nitrobenzamide, |
| 21 | 2-Chloro-N-[2-(4-methoxyphenyl)-5-benzoxazolyl]-5-nitrobenzamide, |
| 22 | 2-Chloro-N-[2-(3-fluorophenyl)-5-benzoxazolyl]-5-nitrobenzamide, |
| 23 | 2-Chloro-N-[2-(2-naphthalenyl)-5-benzoxazolyl]-5-nitrobenzamide, |
| 24 | 2-Chloro-N-[2-[4-(dimethylamino)phenyl]-5-benzoxazolyl]-5-nitrobenzamide, |
| 25 | 2-Chloro-N-[2-[4-(1-methylethyl)phenyl]-5-benzoxazolyl]-5-nitrobenzamide, |
| 26 | 2-Chloro-N-[2-(3-chloro-4-methylphenyl)-5-benzoxazolyl]-5-benzamide |
| 27 | 2-Chloro-N-[2-[4-(1,1-dimethylethyl)phenyl]-5-benzoxazolyl]-5-nitrobenzamide, |
| 28 | 2-Chloro-N-[2-(5-chloro-1-naphthalenyl)-5-benzoxazolyl]-5-nitrobenzamide, |
| 29 | 2-Chloro-N-[2-(3-methoxyphenyl)-5-benzoxazolyl]-5-nitrobenzamide, |
| 30 | N-[2-(3-Bromo-4-methylphenyl)-5-benzoxazolyl]-2-chloro-5-nitrobenzamide, |
| 31 | N-[2-(5-Bromo-1-naphthalenyl)-5-benzoxazolyl]-2-chloro-5-nitrobenzamide, |
| 32 | N-[2-(3-Bromo-4-hydroxyphenyl)-5-benzoxazolyl]-2-chloro-5-nitrobenzamide, |
| 33 | 2-Chloro-N-[2-[4-(diethylamino)phenyl]-5-benzoxazolyl]-5-nitrobenzamide, |
| 34 | 2-Chloro-N-[2-(2,4-dichlorophenyl)-5-benzoxazolyl]-5-nitrobenzamide,, |
| 35 | 2-Chloro-N-[2-(2-chloro-5-iodophenyl)-5-benzoxazolyl]-5-nitrobenzamide |
| 36 | 2-Chloro-N-[2-(3-chloro-4-methoxyphenyl)-5-benzoxazolyl]-5-nitrobenzamide, |
| 37 | N-[2-(2-Bromo-5-iodophenyl)-5-benzoxazolyl]-2-chloro-5-nitrobenzamide, |
| 38 | 2-Chloro-N-[2-(2,3-dichlorophenyl)-5-benzoxazolyl]-5-nitrobenzamide, |
| 39 | N-[2-(5-Bromo-2-chlorophenyl)-5-benzoxazolyl]-2-chloro-5-nitrobenzamide, |
| 40 | N-[2-(3-Bromo-4-methoxyphenyl)-5-benzoxazolyl]-2-chloro-5-nitrobenzamide, |
| 41 | 2-Chloro-N-[2-(2-chloro-4,5-difluorophenyl)-5-benzoxazolyl]-5-nitrobenzamide, |
| 42 | 2-Chloro-5-nitro-N-[2-(2,3,4,5,6-pentafluorophenyl)-5-benzoxazolyl]benzamide, |
| 43 | N-[2-(2-Bromophenyl)-1,3-benzoxazol-5-yl]-2-chloro-5-nitrobenzamide,and |
| 44 | 2-Chloro-N-[3-(5-ethyl-1,3-benzoxazol-2-yl)phenyl]-5-nitrobenzamide |

In one embodiment the invention relates to a method of treatment of cancer, particularly a PPARG activated cancer, such as e.g. certain types of breast cancer, colorectal cancer, esophagael cancer, gastric cancer, hepatocellular cancer, pancreatic cancer, and prostate cancer; and more particularly bladder cancer or pancreatic cancer.

In an embodiment, the invention includes a method of treating a subject diagnosed with a peroxisome proliferator activated receptor gamma (PPARG) activated cancer, the method comprising a step of administering to the subject in need thereof a compound of formula (I) and in addition a step of administering one or more chemotherapeutic agents. In an embodiment, the one or more chemotherapeutic agents are selected from the group consisting of an alkylating agent, an anti-metabolite, an anti-microtubule agent, and a topoisomerase inhibitor.

In an embodiment, the method may further include a step of administering one or more chemotherapeutic agents by intravesical dosing directly into the bladder via an intraurethral catheter. In an embodiment, the one or more chemotherapeutic agents are selected from the group consisting of an alkylating agent, an anti-metabolite, an anti-microtubule agent, and a topoisomerase inhibitor.

In an embodiment, the method may further include a step of administering one or more immune checkpoint inhibitors or immune modulators. Immune checkpoint inhibitors are selected from the group consisting of agents targeting PD1, PD-L1, CTLA4, or immune modulators such as Bacillus Calmette-Guerin (BCG) vaccine, or other modulators.

In an embodiment, the method may further include a step of administering one or more receptor tyrosine kinase inhibitors (RTK). RTK inhibitors are selected from the group consisting of an agents targeting FGFR1, FGFR2, FGFR3, EGFR, ERBB2, or ERBB3.

In an embodiment, the method may further include a step of administering one or more Mitogen-activated protein kinase (MAPK) pathway inhibitors. MAPK inhibitors are selected from the group consisting of an agents targeting KRAS, NRAS, HRAS, BRAF, RAF1 (c-RAF), MAP2K1 (MEK1), MAP2K2 (MEK2), MAPK1 (ERK2), or MAPK3 (ERK1).

### Compound Forms and Salts

Further, it is possible for the compounds of the present invention to exist in free form, e.g. as a free base, or as a free acid, or as a zwitterion, or to exist in the form of a salt. Said salt may be any salt, either an organic or inorganic addition salt, particularly any pharmaceutically acceptable organic or inorganic addition salt, which is customarily used in pharmacy, or which is used, for example, for isolating or purifying the compounds of the present invention.

The term “pharmaceutically acceptable salt” refers to an inorganic or organic acid addition salt of a compound of the present invention. For example, see S. M. Berge, et al. “Pharmaceutical Salts,” J. Pharm. Sci. 1977, 66, 1-19.

A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, or “mineral acid”, such as hydrochloric, hydrobromic, hydroiodic, sulfuric, sulfamic, bisulfuric, phosphoric, or nitric acid, for example, or with an organic acid, such as hydrochloric acid, hydrobromic acid, hydroiodic acid, sulfuric acid, bisulfuric acid, phosphoric acid, and nitric acid or with an organic acid, such as formic acid, acetic acid, acetoacetic acid, pyruvic acid, trifluoroacetic acid, propionic acid, butyric acid, hexanoic acid, heptanoic acid, undecanoic acid, lauric acid, benzoic acid, salicylic acid, 2-(4-hydroxybenzoyl)-benzoic acid, camphoric acid, cinnamic acid, cyclopentanepropionic acid, digluconic acid, 3-hydroxy-2-naphthoic acid, nicotinic acid, pamoic acid, pectinic acid, persulfuric acid, 3-phenylpropionic acid, formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-*2-naphthoic, nicotinic, pamoic, pectinic, 3-phenylpropionic, picric acid, pivalic acid, 2-hydroxyethanesulfonate acid, itaconic acid, sulfamic acid, trifluoromethanesulfonic acid, dodecylsulfuric acid, ethansulfonic acid, benzenesulfonic acid, para-toluenesulfonic acid, methansulfonic acid, 2-naphthalenesulfonic acid, naphthalenedisulfonic acid, camphorsulfonic acid, citric acid, tartaric acid, stearic acid, lactic acid, oxalic acid, malonic acid, succinic acid, malic acid, adipic acid, alginic acid, maleic acid, fumaric acid, D-gluconic acid, mandelic acid, ascorbic acid, glucoheptanoic acid, glycerophosphoric acid, aspartic acid, sulfosalicylic acid, hemisulfuric acid and thiocyanic acid for example.

A “pharmaceutically acceptable anion” refers to the deprotonated form of a conventional acid, such as, for example, a hydroxide, a carboxylate, a sulfate, a halide, a phosphate, or a nitrate.

Physiologically acceptable salts of the compounds according to the invention also comprise salts of conventional bases, such as, by way of example and by preference, alkali metal salts (for example lithium, sodium and potassium salts), alkaline earth metal salts (for example calcium, strontium and magnesium salts) or an aluminium salt or a zinc salt, or an ammonium salt derived from ammonia or from an organic primary, secondary or tertiary amine having 1 to 20 carbon atoms, such as by way of example ethylamine, diethylamine, triethylamine, ethyldiisopropylamine, monoethanolamine, diethanolamine, triethanolamine, dicyclohexylamine, dimethylaminoethanol, diethylaminoethanol, tris(hydroxymethyl)aminomethane, procaine, dibenzylamine, N-methylmorpholine, arginine, lysine, 1,2-ethylenediamine, N-methylpiperidine, N-methyl-glucamine, N,N-dimethyl-glucamine, N-ethyl-glucamine, 1,6-hexanediamine, glucosamine, sarcosine, serinol, 2-amino-1,3-propanediol, 3-amino-1,2-propanediol, 4-amino-1,2,3-butanetriol.

Additionally, the compounds according to the invention may form salts with a quaternary ammonium ion obtainable, e.g., by quaternisation of a basic nitrogen-containing group with agents such as lower alkylhalides, such as alkylchlorides, e.g. methylchloride, ethylchloride, propylchloride and butylchloride; such as alkylbromides, e.g. methylbromide, ethylbromide, propylbromide and butylbromide; and such as alkyliodides; e.g. methyliodide, ethyliodide, propyliodide and butyliodide; dialkylsulfates such as dimethylsulfate, diethylsulfate, dibutylsulfate and diamylsulfates, long chain halides such as e.g. decylchloride, laurylchloride, myristylchloride and stearylchloride, decylbromide, laurylbromide, myristylbromide and stearylbromide, decyliodide, lauryliodide, myristyliodide and stearyliodide, aralkylhalides such as benzylchloride, benzylbromide, benzyliodide and phenethylbromides and others. Examples of suitable quaternary ammonium ions are tetramethylammonium, tetraethylammonium, tetra(n-propyl)ammonium, tetra (n-butyl)ammonium, or N-benzyl-N,N,N-trimethylammonium.

Those skilled in the art will further recognise that it is possible for acid addition salts of the claimed compounds to be prepared by reaction of the compounds with the appropriate inorganic or organic acid via any of a number of known methods. Alternatively, alkali and alkaline earth metal salts of acidic compounds of the present invention are prepared by reacting the compounds of the present invention with the appropriate base via a variety of known methods.

The present invention includes all possible salts of the compounds of the present invention as single salts, or as any mixture of said salts, in any ratio.

In the present text, in particular in the Experimental Section, for the synthesis of intermediates and of examples of the present invention, when a compound is mentioned as a salt form with the corresponding base or acid, the exact stoichiometric composition of said salt form, as obtained by the respective preparation and/or purification process, is, in most cases, unknown.

Unless specified otherwise, suffixes to chemical names or structural formulae relating to salts, such as “hydrochloride”, “trifluoroacetate”, “sodium salt”, or “x HCl”, “x CF3COOH”, “x Na+”, for example, mean a salt form, the stoichiometry of which salt form not being specified.

This applies analogously to cases in which synthesis intermediates or example compounds or salts thereof have been obtained, by the preparation and/or purification processes described, as solvates, such as hydrates, with (if defined) unknown stoichiometric composition.

The compounds of the present invention can exist as a hydrate, or as a solvate, wherein the compounds of the present invention contain polar solvents, in particular water, methanol or ethanol for example, as structural element of the crystal lattice of the compounds. It is possible for the amount of polar solvents, in particular water, to exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

The present invention also includes various hydrate and solvate forms of the compounds.

The compounds of the present invention optionally contain one or more asymmetric centres, depending upon the location and nature of the various substituents desired, which are e.g. carbon atoms having four different substituents. It is possible that one or more asymmetric carbon atoms are present in the (R) or (S) configuration, which can result in racemic mixtures in the case of a single asymmetric centre, and in diastereomeric mixtures in the case of multiple asymmetric centres. The term “(±)” is used to designate a racemic mixture where appropriate. In certain instances, it is possible that asymmetry also be present due to restricted rotation about a given bond, for example, the central bond adjoining two substituted aromatic rings of the specified compounds. When a compound is an enantiomer, the stereochemistry at each chiral carbon can be specified by either (R) or (S). Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and can thus give rise to enantiomers, diastereomers, and other stereoisomeric forms that can be defined, in terms of absolute stereochemistry at each asymmetric atom, as (R)- or (S)-. The present chemical entities, pharmaceutical compositions and methods are meant to include all such possible isomers, including racemic mixtures, optically substantially pure forms and intermediate mixtures.

Preferred compounds are those which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art including chiral high pressure liquid chromatography (HPLC), the formation and crystallization of chiral salts, or prepared by asymmetric syntheses.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Preferred is the stereoisomer which shows the desired effect. For compounds of formula (I) wherein R⁴=methyl it is discovered that the compounds having said methyl group in the S-configuration do have a significantly better pharmacological effect.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope which is higher than its natural abundance. The natural abundances of isotopes to be applied in this context are described in “Isotopic Compositions of the Elements 1997”, Pure Appl. Chem., 70(1), 217-235, 1998.

Examples of such isotopes include stable and radioactive isotopes of hydrogen, carbon, nitrogen, oxygen, phosphorus, sulfur, fluorine, chlorine, bromine and iodine, such as 2H (deuterium), 3H (tritium), 11C, 13C, 14C, 15N, 17O, 18O, 32P, 33P, 33S, 34S, 35S, 36S, 18F, 36Cl, 82Br, 123I, 124I, 125I, 129I and 131I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as 3H or 14C, are incorporated are useful e.g. in drug and/or substrate tissue distribution studies. These isotopes are particularly preferred for the ease of their incorporation and detectability. Positron emitting isotopes such as 18F or 11C may be incorporated into a compound of general formula (I). These isotopic variants of the compounds of general formula (I) are useful for in vivo imaging applications. Deuterium-containing and 13C-containing compounds of general formula (I) can be used in mass spectrometry analyses (H. J. Leis et al., Curr. Org. Chem., 1998, 2, 131) in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D20 can be incorporated either directly into the compounds or into reagents that are useful for synthesizing such compounds (Esaki et al., Tetrahedron, 2006, 62, 10954; Esaki et al., Chem. Eur. J., 2007, 13, 4052). Deuterium gas is also a useful reagent for incorporating deuterium into molecules. Catalytic deuteration of olefinic bonds (H. J. Leis et al., Curr. Org. Chem., 1998, 2, 131; J. R. Morandi et al., J. Org. Chem., 1969, 34 (6), 1889) and acetylenic bonds (N. H. Khan, J. Am. Chem. Soc., 1952, 74 (12), 3018; S. Chandrasekhar et al., Tetrahedron Letters, 2011, 52, 3865) is a rapid route for incorporation of deuterium. Metal catalysts (i.e. Pd, Pt, and Rh) in the presence of deuterium gas can be used to directly exchange deuterium for hydrogen in functional groups containing hydrocarbons (J. G. Atkinson et al., U.S. Pat. No. 3,966,781). A variety of deuterated reagents and synthetic building blocks are commercially available from companies such as for example C/D/N Isotopes, Quebec, Canada; Cambridge Isotope Laboratories Inc., Andover, MA, USA; and CombiPhos Catalysts, Inc., Princeton, NJ, USA. Further information on the state of the art with respect to deuterium-hydrogen exchange is given for example in Hanzlik et al., J. Org. Chem. 55, 3992-3997, 1990; R. P. Hanzlik et al., Biochem. Biophys. Res. Commun. 160, 844, 1989; P. J. Reider et al., J. Org. Chem. 52, 3326-3334, 1987; M. Jarman et al., Carcinogenesis 16(4), 683-688, 1995; J. Atzrodt et al., Angew. Chem., Int. Ed. 2007, 46, 7744; K. Matoishi et al., Chem. Commun. 2000, 1519-1520; K. Kassahun et al., WO2012/112363.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490; A. Streitwieser et al., J. Am. Chem. Soc., 1963, 85, 2759;], basicity [C. L. Perrin et al., J. Am. Chem. Soc., 2005, 127, 9641; C. L. Perrin, et al., J. Am. Chem. Soc., 2003, 125, 15008; C. L. Perrin in Advances in Physical Organic Chemistry, 44, 144], lipophilicity [B. Testa et al., Int. J. Pharm., 1984, 19(3), 271]) and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102; D. J. Kushner et al., Can. J. Physiol. Pharmacol., 1999, 77, 79). These changes in the exposure to parent drug and metabolites can have important consequences with respect to the pharmacodynamics, tolerability and efficacy of a deuterium-containing compound of general formula (I). In some cases deuterium substitution reduces or eliminates the formation of an undesired or toxic metabolite and enhances the formation of a desired metabolite (e.g. Nevirapine: A. M. Sharma et al., Chem.

Res. Toxicol., 2013, 26, 410; Efavirenz: A. E. Mutlib et al., Toxicol. Appl. Pharmacol., 2000, 169, 102). In other cases the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208) and Odanacatib (K. Kassahun et al., WO2012/112363) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch. /Drug. Res., 2006, 56, 295; Telaprevir: F. Maltais et al., J. Med. Chem., 2009, 52, 7993). Deuterated drugs showing this effect may have reduced dosing requirements (e.g. lower number of doses or lower dosage to achieve the desired effect) and/or may produce lower metabolite loads.

A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P450.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention optionally contain one or more asymmetric centres, depending upon the location and nature of the various substituents desired. It is possible that one or more asymmetric carbon atoms are present in the (R) or (S) configuration, which can result in racemic mixtures in the case of a single asymmetric centre, and in diastereomeric mixtures in the case of multiple asymmetric centres. In certain instances, it is possible that asymmetry also be present due to restricted rotation about a given bond, for example, the central bond adjoining two substituted aromatic rings of the specified compounds.

One embodiment includes those compounds which produce the more desirable biological activity. Separated, pure or partially purified isomers and stereoisomers or racemic or diastereomeric mixtures of the compounds of the present invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., HPLC columns using a chiral phase), with or without conventional derivatisation, optimally chosen to maximise the separation of the enantiomers. Suitable HPLC columns using a chiral phase are commercially available, such as those manufactured by Daicel, e.g., Chiracel OD and Chiracel OJ, for example, among many others, which are all routinely selectable. Enzymatic separations, with or without derivatisation, are also useful. The optically active compounds of the present invention can likewise be obtained by chiral syntheses utilizing optically active starting materials.

In order to distinguish different types of isomers from each other reference is made to IUPAC Rules Section E (Pure Appl Chem 45, 11-30, 1976).

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example.

The present invention also includes useful forms of the compounds of the present invention, such as metabolites, hydrates, solvates, prodrugs, salts, in particular pharmaceutically acceptable salts, and/or co-precipitates.

### DESCRIPTION

In some embodiments the invention includes compounds of formula (I) wherein, independently from each occurrence,
L is selected from R

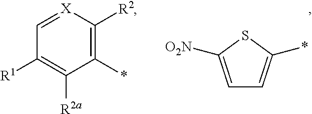

R¹ is selected from NO₂, a cyano group, a —C═(O)NH—CH₃ group, a —C═(O)NH—CH═CH₂ group and CF₃
R² is selected from a hydrogen atom, a fluorine atom, a chlorine atom, a bromine atom and an iodine atom;
R^{2a} is a hydrogen atom;
X is a CR³ group and a nitrogen atom
R³ is a hydrogen atom;
A is selected from the groups (a) to (h)

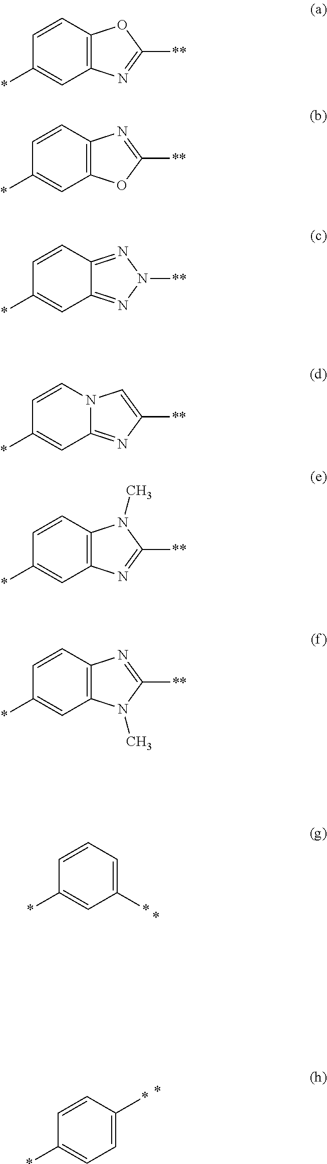

whereby * marks the position where ring A is bound to the nitrogen atom of the amide and ** marks the position where ring A is bound to ring B;
B is selected from the groups (i) and (j)

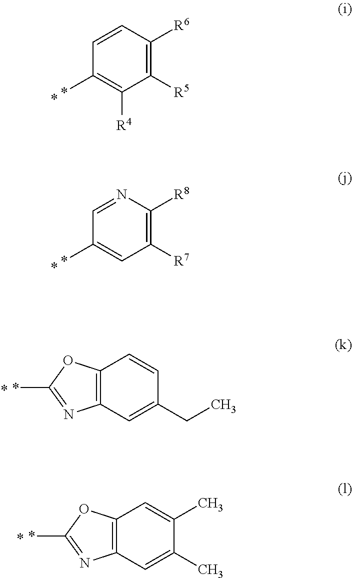

whereby ** is the point of attachment to the A ring;
R⁴ is selected from the group consisting of a hydrogen atom, a bromine atom and a methyl group;
R⁵ is selected from the group consisting of a hydrogen atom, a fluorine atom, a chlorine atom, and a methyl group;
R⁶ is selected from the group consisting of a hydrogen atom, a methyl group, an ethyl group, a methoxy group, and a group

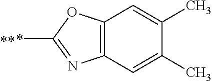 [SMILES: *c1nc2cc(C)c(C)cc2o1]

whereby the *** marks the point of attachment to the phenyl ring;
R⁷ is selected from the group consisting of a hydrogen atom and a methyl group;
R⁸ is selected from the group consisting of a hydrogen atom and an ethyl group; with the proviso, that those compounds are excluded for which
   R¹ is a nitro group,
   R² is a chlorine atom,
   R^{2a} is a hydrogen atom,
   X is a CH group,
   ring A is a benzoxazole (a),
   R⁴ is selected from the group consisting of a hydrogen atom, a halogen atom and a methyl group,
   R⁵ is selected from the group consisting of a hydrogen atom, a fluorine atom, a chlorine atom, a methyl group and
   R⁶ is selected from the group consisting of a hydrogen atom, a methyl group, an ethyl group, a methoxy group,
or a stereoisomer, a tautomer, an N-oxide, a hydrate, a solvate, or a salt thereof, or a mixture of same.

In yet other embodiments the invention includes compounds of formula (I) selected from the group consisting of
2-Chloro-N-[2-(3-methylphenyl)-1,3-benzoxazol-5-yl]-5-nitrobenzamide,
2-Chloro-N-[2-(2-methylphenyl)-1,3-benzoxazol-5-yl]-5-nitrobenzamide,
2-Chloro-N-[2-(4-ethylphenyl)-1,3-benzoxazol-5-yl]-5-nitrobenzamide,
2-Chloro-N-[2-(3-chlorophenyl)-1,3-benzoxazol-5-yl]-5-nitrobenzamide,
2-Chloro-N-[2-(3-fluoro-4-methoxyphenyl)-1,3-benzoxazol-5-yl]-5-nitrobenzamide,
2-Chloro-N-[2-(5-methylpyridin-3-yl)-1,3-benzoxazol-5-yl]-5-nitrobenzamide,
2-Chloro-N-[2-(6-ethylpyridin-3-yl)-1,3-benzoxazol-5-yl]-5-nitrobenzamide,
2-Chloro-N-[2-(4-methylphenyl)-1,3-benzoxazol-6-yl]-5-nitrobenzamide,
2-Chloro-N-[2-(4-ethylphenyl)-1-methyl-1H-benzimidazol-5-yl]-5-nitrobenzamide,
2-Chloro-N-[2-(4-ethylphenyl)-1-methyl-1H-benzimidazol-6-yl]-5-nitrobenzamide,
2-Chloro-5-nitro-N-(2-phenylimidazo[1,2-a]pyridin-6-yl)benzamide,
2-Chloro-N-[2-(4-methylphenyl)-2H-benzotriazol-5-yl]-5-nitrobenzamide,
2-Chloro-N-[2-(4-ethylphenyl)-1,3-benzoxazol-5-yl]-5-nitrobenzene-1-sulfonamide, 2-chloro-N-[4-(5,6-dimethyl-1,3-benzoxazol-2-yl)phenyl]-5-nitrobenzamide,
2-Chloro-N-[2-(4-ethylphenyl)-1,3-benzoxazol-5-yl]-5-(trifluoromethyl) benzamide, 4-Chloro-N³-[2-(4-ethylphenyl)-1,3-benzoxazol-5-yl]-N¹-methylbenzene-1,3-dicarboxamide,
2-Chloro-N-[2-(4-ethylphenyl)-1,3-benzoxazol-5-yl]pyridine-3-carboxamide,
2-chloro-5-cyano-N-[2-(4-ethylphenyl)-1,3-benzoxazol-5-yl]benzamide,
N-[2-(4-Ethylphenyl)-1,3-benzoxazol-5-yl]-3-nitrobenzamide,
2-Chloro-N-[2-(3-methylphenyl)-1,3-benzoxazol-5-yl]-3-nitrobenzamide,
N-[2-(4-Ethylphenyl)-1,3-benzoxazol-5-yl]-5-nitrothiophene-2-carboxamide,
N-[2-(4-Ethylphenyl)-1,3-benzoxazol-5-yl]prop-2-enamide,
N-[2-(4-Ethylphenyl)-1,3-benzoxazol-5-yl]-2-fluoro-5-nitrobenzamide, 2-Bromo-N-[2-(4-ethylphenyl)-1,3-benzoxazol-5-yl]-5-nitrobenzamide, and
N-[2-(4-ethylphenyl)-1,3-benzoxazol-5-yl]-2-iodo-5-nitrobenzamide,
   or a stereoisomer, a tautomer, an N-oxide, a hydrate, a solvate, or a salt thereof, or a mixture of same.

In other embodiments the invention relates to the compound of formula (I) which is 2-Bromo-N-[2-(4-ethylphenyl)-1,3-benzoxazol-5-yl]-5-nitrobenzamide or a stereoisomer, a tautomer, an N-oxide, a hydrate, a solvate, or a salt thereof, or a mixture of same.

In other embodiments the invention relates to the compound of formula (I) which is 2-Chloro-N-[2-(4-ethylphenyl)-1,3-benzoxazol-5-yl]-5-nitrobenzene-1-sulfonamide or a stereoisomer, a tautomer, an N-oxide, a hydrate, a solvate, or a salt thereof, or a mixture of same.

In further embodiments the invention includes compounds of formula (I) selected from the group consisting of
2-Chloro-N-[2-(3-methylphenyl)-1,3-benzoxazol-5-yl]-5-nitrobenzamide,
2-Chloro-N-[2-(2-methylphenyl)-1,3-benzoxazol-5-yl]-5-nitrobenzamide,
2-Chloro-N-[2-(4-ethylphenyl)-1,3-benzoxazol-5-yl]-5-nitrobenzamide,
2-Chloro-N-[2-(3-chlorophenyl)-1,3-benzoxazol-5-yl]-5-nitrobenzamide,
2-Chloro-N-[2-(3-fluoro-4-methoxyphenyl)-1,3-benzoxazol-5-yl]-5-nitrobenzamide,
2-Chloro-N-[2-(5-methylpyridin-3-yl)-1,3-benzoxazol-5-yl]-5-nitrobenzamide,
2-Chloro-N-[2-(6-ethylpyridin-3-yl)-1,3-benzoxazol-5-yl]-5-nitrobenzamide,
2-Chloro-N-[2-(4-methylphenyl)-1,3-benzoxazol-6-yl]-5-nitrobenzamide,
2-Chloro-N-[2-(4-ethylphenyl)-1-methyl-1H-benzimidazol-5-yl]-5-nitrobenzamide,
2-Chloro-N-[2-(4-ethylphenyl)-1-methyl-1H-benzimidazol-6-yl]-5-nitrobenzamide,
2-Chloro-5-nitro-N-(2-phenylimidazo[1,2-a]pyridin-6-yl)benzamide,
2-Chloro-N-[2-(4-methylphenyl)-2H-benzotriazol-5-yl]-5-nitrobenzamide,
2-chloro-N-[4-(5,6-dimethyl-1,3-benzoxazol-2-yl)phenyl]-5-nitrobenzamide,
2-Chloro-N-[2-(4-ethylphenyl)-1,3-benzoxazol-5-yl]-5-(trifluoromethyl) benzamide,
4-Chloro-N³-[2-(4-ethylphenyl)-1,3-benzoxazol-5-yl]-N¹-methylbenzene-1,3-dicarboxamide,
2-Chloro-N-[2-(4-ethylphenyl)-1,3-benzoxazol-5-yl]pyridine-3-carboxamide,
2-chloro-5-cyano-N-[2-(4-ethylphenyl)-1,3-benzoxazol-5-yl]benzamide,
N-[2-(4-Ethylphenyl)-1,3-benzoxazol-5-yl]-3-nitrobenzamide,
2-Chloro-N-[2-(3-methylphenyl)-1,3-benzoxazol-5-yl]-3-nitrobenzamide,
N-[2-(4-Ethylphenyl)-1,3-benzoxazol-5-yl]-5-nitrothiophene-2-carboxamide,
N-[2-(4-Ethylphenyl)-1,3-benzoxazol-5-yl]prop-2-enamide,
N-[2-(4-Ethylphenyl)-1,3-benzoxazol-5-yl]-2-fluoro-5-nitrobenzamide,
2-Bromo-N-[2-(4-ethylphenyl)-1,3-benzoxazol-5-yl]-5-nitrobenzamide, and
N-[2-(4-ethylphenyl)-1,3-benzoxazol-5-yl]-2-iodo-5-nitrobenzamide,
   or a stereoisomer, a tautomer, an N-oxide, a hydrate, a solvate, or a salt thereof, or a mixture of same.

In other embodiments the invention relates to compounds of formula (I

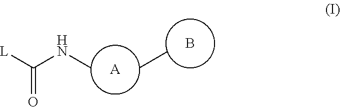

wherein,
L is selected from

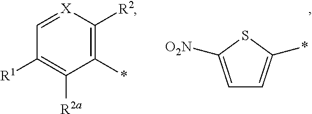

and an ethenylene group
R¹ is selected from a hydrogen atom, a NO₂ group, a cyano group, a —C═(O)NH—(C₁-C₃-alkyl) group, and C₁-C₃-haloalkyl group;
R² is selected from a hydrogen atom and a halogen atom;
R^{2a} is a hydrogen atom;
X is a CR³ group and a nitrogen atom
R³ is a hydrogen atom;
Ring A is selected from the groups (a) to (h)

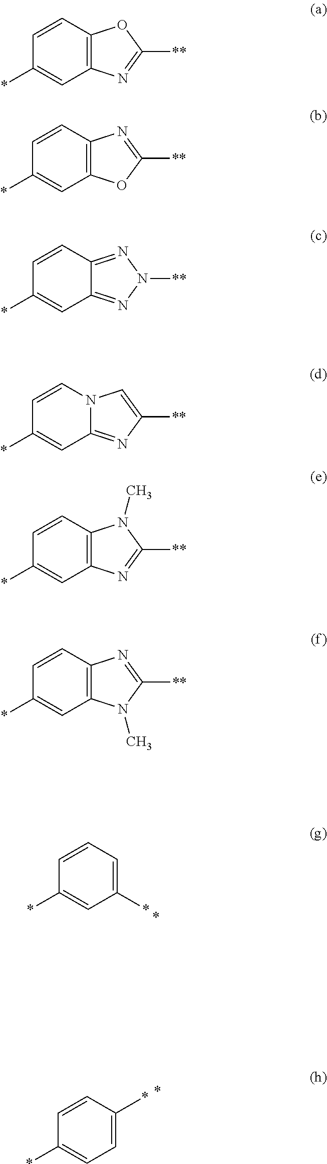

whereby * marks the position where ring A is bound to the nitrogen atom of the amide and ** marks the position where ring A is bound to ring B;
B is selected from the groups

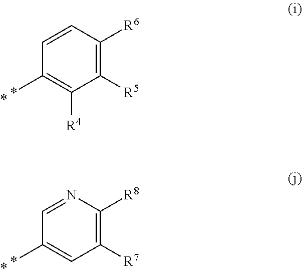

whereby ** is the point of attachment to the A ring,
R⁴ is selected from the group consisting of a hydrogen atom, a halogen atom and a C₁-C₃-alkyl group;
R⁵ is selected from the group consisting of a hydrogen atom, a halogen atom, and a C₁-C₃-alkyl group;
R⁶ is selected from the group consisting of a hydrogen atom, a C₁-C₃-alkyl group, and a C₁-C₃-alkoxy group,
R⁷ is selected form the group consisting of a hydrogen atom and a C₁-C₃-alkyl group;
R⁸ is selected form the group consisting of a hydrogen atom and a C₁-C₃-alkyl group; with the proviso, that those compounds are excluded for which
   R¹ is a nitro group,
   R² is a chlorine atom,
   R^{2a} is a hydrogen atom,
   X is a CH group,
   ring A is a benzoxazole (a) or (b),
   R⁴ is selected from the group consisting of a hydrogen atom, a halogen atom and a C₁-C₃-alkyl group,
   R⁵ is selected from the group consisting of a hydrogen atom, a halogen atom, a C₁-C₃-alkyl group and
   R⁶ is selected from the group consisting of a hydrogen atom, a C₁-C₃-alkyl group, a C₁-C₃-alkoxy group,
or a stereoisomer, a tautomer, an N-oxide, a hydrate, a solvate, or a salt thereof, or a mixture of same.

In other embodiments the invention relates to compounds of formula (I)

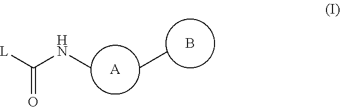

wherein,
L is selected from

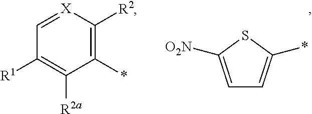

and an ethenylene group
R¹ is selected from a hydrogen atom, a NO₂ group, a cyano group, a —C═(O)NH—(C₁-C₃-alkyl) group, and C₁-C₃-haloalkyl group;
R² is selected from a hydrogen atom and a halogen atom;
R^{2a} is a hydrogen atom;
X is a CR³ group and a nitrogen atom
R³ is a hydrogen atom;
Ring A is selected from the groups (a) to (f)

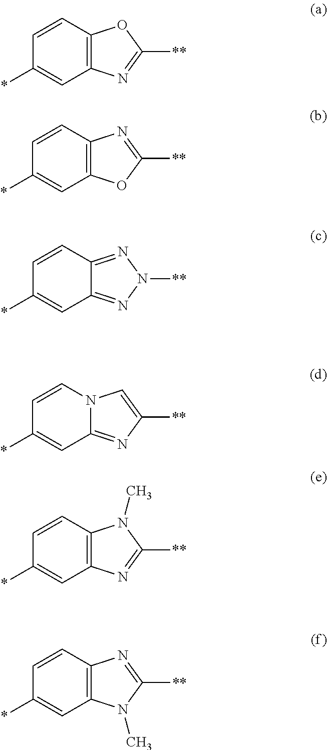

whereby * marks the position where ring A is bound to the nitrogen atom of the amide and ** marks the position where ring A is bound to ring B;
B is selected from the groups

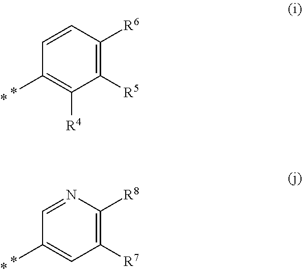

whereby ** is the point of attachment to the A ring,
R⁴ is selected from the group consisting of a hydrogen atom, a halogen atom and a C₁-C₃-alkyl group;
R⁵ is selected from the group consisting of a hydrogen atom, a halogen atom, and a C₁-C₃-alkyl group;
R⁶ is selected from the group consisting of a hydrogen atom, a C₁-C₃-alkyl group, and a C₁-C₃-alkoxy group,
R⁷ is selected form the group consisting of a hydrogen atom and a C₁-C₃-alkyl group;
R⁸ is selected form the group consisting of a hydrogen atom and a C₁-C₃-alkyl group;
   with the proviso, that those compounds are excluded for which
   R¹ is a nitro group,
   R² is a chlorine atom,
   R^{2a} is a hydrogen atom,
   X is a CH group,
   ring A is a benzoxazole (a) or (b),
   R⁴ is selected from the group consisting of a hydrogen atom, a halogen atom and a C₁-C₃-alkyl group,
   R⁵ is selected from the group consisting of a hydrogen atom, a halogen atom, a C₁-C₃-alkyl group and
   R⁶ is selected from the group consisting of a hydrogen atom, a C₁-C₃-alkyl group, a C₁-C₃-alkoxy group,
or a stereoisomer, a tautomer, an N-oxide, a hydrate, a solvate, or a salt thereof, or a mixture of same.

In other embodiments the invention relates to compounds of formula (I),
wherein, independently from each occurrence
R¹ is selected from a hydrogen atom, a NO₂ group, a cyano group, a —C═(O)NH—CH₃ group, and a CF₃ group;
R² is selected from a hydrogen atom, a fluorine atom, a chlorine atom, a bromine atom and an iodine atom;
R^{2a} is a hydrogen atom;
X is a CR³ group and a nitrogen atom;
R³ is a hydrogen atom;
A is selected from the groups (a) to (h)

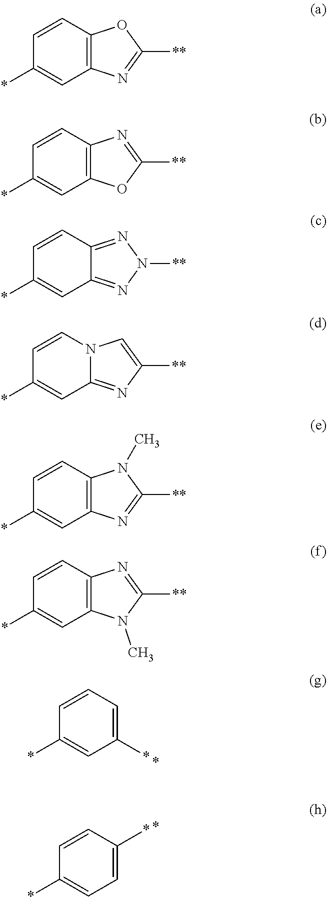

whereby * marks the position where ring A is bound to the nitrogen atom of the amide and ** marks the position where ring A is bound to ring B;
B is selected from the groups (i) and (I)

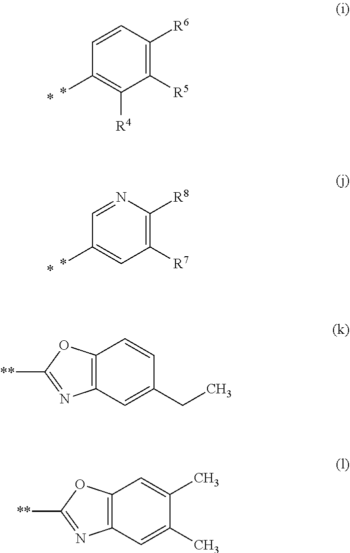

whereby ** ist the point of attachment to the A ring;
R⁴ is selected from the group consisting of a hydrogen atom, a bromine atom and a methyl group;
R⁵ is selected from the group consisting of a hydrogen atom, a fluorine atom, a chlorine atom, and a methyl group
R⁶ is selected from the group consisting of a hydrogen atom, a methyl group, an ethyl group, and a methoxy group,
R⁷ is a hydrogen atom or a methyl group;
R⁸ is a hydrogen atom or an ethyl group;
   with the proviso, that those compounds are excluded for which
   R¹ is a nitro group,
   R² is a chlorine atom,
   R^{2a} is a hydrogen atom,
   X is a CH group,
   ring A is a benzoxazole (a) or (b),
   R⁴ is selected from the group consisting of a hydrogen atom, a halogen atom and a methyl group,
   R⁵ is selected from the group consisting of a hydrogen atom, a fluorine atom, a chlorine atom, a methyl group and
   R⁶ is selected from the group consisting of a hydrogen atom, a methyl group, a methoxy group,
or a stereoisomer, a tautomer, an N-oxide, a hydrate, a solvate, or a salt thereof, or a mixture of same.

In other embodiments the invention relates to compounds of formula (I),
wherein, independently from each occurrence
R¹ is selected from a hydrogen atom, a NO₂ group, a cyano group, a —C═(O)NH—CH₃ group, and a CF₃ group;
R² is selected from a hydrogen atom, a fluorine atom, a chlorine atom, a bromine atom and an iodine atom;
R^{2a} is a hydrogen atom;
X is a CR³ group and a nitrogen atom;
R³ is a hydrogen atom;
A is selected from the groups (a) to (f)

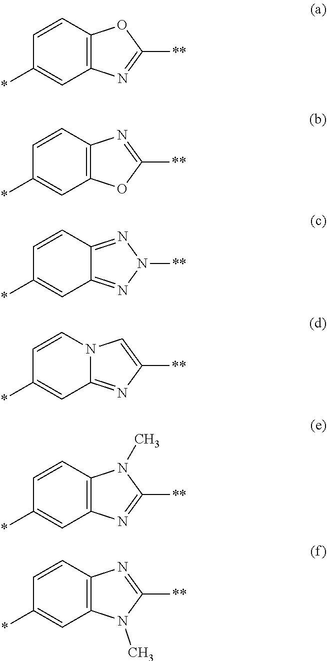

whereby * marks the position where ring A is bound to the nitrogen atom of the amide and ** marks the position where ring A is bound to ring B;
B is selected from the groups (i) and (I)

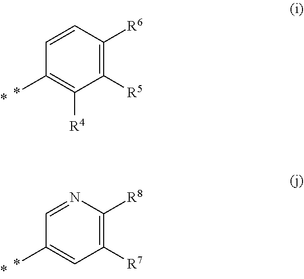

whereby ** ist the point of attachment to the A ring;
R⁴ is selected from the group consisting of a hydrogen atom, a bromine atom and a methyl group;
R⁵ is selected from the group consisting of a hydrogen atom, a fluorine atom, a chlorine atom, and a methyl group
R⁶ is selected from the group consisting of a hydrogen atom, a methyl group, an ethyl group, and a methoxy group,
R⁷ is a hydrogen atom or a methyl group;
R⁸ is a hydrogen atom or an ethyl group;
   with the proviso, that those compounds are excluded for which
   R¹ is a nitro group,
   R² is a chlorine atom,
   R^{2a} is a hydrogen atom,
   X is a CH group,
   ring A is a benzoxazole (a) or (b),
   R⁴ is selected from the group consisting of a hydrogen atom, a halogen atom and a methyl group,
   R⁵ is selected from the group consisting of a hydrogen atom, a fluorine atom, a chlorine atom, a methyl group and
   R⁶ is selected from the group consisting of a hydrogen atom, a methyl group, a methoxy group,
or a stereoisomer, a tautomer, an N-oxide, a hydrate, a solvate, or a salt thereof, or a mixture of same.

In some embodiments the invention relates to compounds of formula (I), wherein R¹ is selected from a hydrogen atom, a NO₂ group, a cyano group, a —C═(O)NH—(C₁-C₃-alkyl) group, and C₁-C₃-haloalkyl group.

In some embodiments the invention relates to compounds of formula (I), wherein R¹ is a hydrogen atom.

In yet other embodiments the invention relates to compounds of formula (I) wherein R¹ is a NO₂ group, a cyano group, a —C═(O)NH—(C₁-C₃-alkyl) group, and C₁-C₃-haloalkyl group.

In other embodiments the invention relates to compounds of formula (I) wherein R¹ is a cyano group, a —C═(O)NH—(C₁-C₃-alkyl) group, and C₁-C₃-haloalkyl group.

In other embodiments the invention relates to compounds of formula (I) wherein R¹ is a hydrogen atom, a cyano group, a —C═(O)NH—(C₁-C₃-alkyl) group, and C₁-C₃-haloalkyl group.

In other embodiments the invention relates to compounds wherein R¹ is selected from a hydrogen atom, NO₂, a cyano group, a —C═(O)NH—CH₃ group, and a CF₃ group.

In other embodiments the invention relates to compounds wherein R¹ is selected from a NO₂ group, a cyano group, a —C═(O)NH—CH₃ group, and a CF₃ group.

In other embodiments the invention relates to compounds wherein R¹ is selected from a hydrogen atom, a cyano group, a —C═(O)NH—CH₃ group, and a CF₃ group.

In other embodiments the invention relates to compounds wherein R¹ is selected from a cyano group, a —C═(O)NH—CH₃ group, and a CF₃ group.

In further embodiments the invention relates to compounds of formula (I) wherein R² is selected from hydrogen atom and a halogen atom.

In further embodiments the invention relates to compounds of formula (I) wherein R² is a halogen atom.

In further embodiments the invention relates to compounds of formula (I) wherein R² is a hydrogen atom.

In further embodiments the invention relates to compounds of formula (I) wherein R² is selected from a hydrogen atom, a fluorine atom, a chlorine atom, a bromine atom and an iodine atom.

In further embodiments the invention relates to compounds of formula (I) wherein R² is selected from a fluorine atom, a chlorine atom, a bromine atom and an iodine atom.

In further embodiments the invention relates to compounds of formula (I) wherein R² is selected from a fluorine atom, a bromine atom and an iodine atom.

In further embodiments the invention relates to compounds of formula (I) wherein R² is a chlorine atom.

In further embodiments the invention relates to compounds of formula (I) wherein R² is selected from a bromine atom and an iodine atom.

In further embodiments the invention relates to compounds of formula (I) wherein R^{2a} is a hydrogen atom or a halogen atom.

In further embodiments the invention relates to compounds of formula (I) wherein R^{2a} is a halogen atom.

In further embodiments the invention relates to compounds of formula (I) wherein R^{2a} is a hydrogen atom or a chlorine atom.

In further embodiments the invention relates to compounds of formula (I) wherein R^{2a} is a chlorine atom.

In further embodiments the invention relates to compounds of formula (I) wherein R^{2a} is a hydrogen atom.

In other embodiments the invention relates to compounds of formula (I) wherein R³ is a hydrogen atom, or a halogen atom.

In other embodiments the invention relates to compounds of formula (I) wherein R³ is a hydrogen atom.

In other embodiments the invention relates to compounds of formula (I) wherein R³ is a halogen atom.

In other embodiments the invention relates to compounds of formula (I) wherein R³ is a hydrogen atom, or chlorine atom.

In other embodiments the invention relates to compounds of formula (I) wherein R³ is a chlorine atom.

In some embodiments the invention relates to compounds of formula (I) wherein R² is a fluorine atom, a chlorine atom, a bromine atom or a iodine atom and R¹ is a NO₂ group.

In some embodiments the invention relates to compounds of formula (I) wherein R² is a a chlorine atom and R¹ is a NO₂ group.

In further embodiments the invention relates to compounds of formula (I) wherein R⁴ is selected from a hydrogen atom, a halogen atom and a C₁-C₃-alkyl group.

In further embodiments the invention relates to compounds of formula (I) wherein R⁴ is a hydrogen atom.

In further embodiments the invention relates to compounds of formula (I) wherein R⁴ is selected from a halogen atom and a C₁-C₃-alkyl group.

In further embodiments the invention relates to compounds of formula (I) wherein R⁴ is a a C₁-C₃-alkyl group.

In further embodiments the invention relates to compounds of formula (I) wherein R⁴ is a bromine atom.

In further embodiments the invention relates to compounds of formula (I) wherein R⁴ is selected from a hydrogen atom, a bromine atom and a methyl group.

In further embodiments the invention relates to compounds of formula (I) wherein R⁴ is selected from a bromine atom and a methyl group.

In further embodiments the invention relates to compounds of formula (I) wherein R⁴ is selected from a hydrogen atom, and a bromine atom.

In further embodiments the invention relates to compounds of formula (I) wherein R⁴ is selected from a hydrogen atom, and a methyl group.

In further embodiments the invention relates to compounds of formula (I) wherein R⁵ is selected from a hydrogen atom, a halogen atom, and a C₁-C₃-alkyl group.

In further embodiments the invention relates to compounds of formula (I) wherein R⁵ is a hydrogen atom.

In further embodiments the invention relates to compounds of formula (I) wherein R⁵ is selected from a hydrogen atom, a and C₁-C₃-alkyl group.

In further embodiments the invention relates to compounds of formula (I) wherein R⁵ is selected from a hydrogen atom, a fluorine atom, a chlorine atom, and a methyl group.

In further embodiments the invention relates to compounds of formula (I) wherein R⁵ is a hydrogen atom.

In other embodiments the invention relates compounds of formula (I) wherein R⁶ is selected from is a hydrogen atom or a C₁-C₃-alkyl group.

In other embodiments the invention relates compounds of formula (I) wherein R⁶ is selected from a hydrogen atom, a C₁-C₃-alkyl group, a C₁-C₃-alkoxy group.

In other embodiments the invention relates compounds of formula (I) wherein R⁶ is selected from a C₁-C₃-alkyl group, a C₁-C₃-alkoxy group.

In other embodiments the invention relates compounds of formula (I) wherein R⁶ is selected from is a hydrogen atom or a C₁-C₃-alkoxy group.

In other embodiments the invention relates compounds of formula (I) wherein R⁶ is a hydrogen atom.

In further embodiments the invention relates compounds of formula (I) wherein R⁷. is selected from a hydrogen atom and a C₁-C₃-alkyl group.

In further embodiments the invention relates compounds of formula (I) wherein R⁷. is a hydrogen atom.

In further embodiments the invention relates compounds of formula (I) wherein R⁷. is selected from a C₁-C₃-alkyl group.

In further embodiments the invention relates compounds of formula (I) wherein R⁷. is selected from a hydrogen atom and a methyl group.

In further embodiments the invention relates compounds of formula (I) wherein R⁷. is selected from a methyl group.

In further embodiments the invention relates compounds of formula (I) wherein R⁸ is selected from a hydrogen atom and a C₁-C₃-alkyl group.

In further embodiments the invention relates compounds of formula (I) wherein R⁸ is a hydrogen atom.

In further embodiments the invention relates compounds of formula (I) wherein R⁸ is a C₁-C₃-alkyl group.

In further embodiments the invention relates compounds of formula (I) wherein R⁸ is selected from a hydrogen atom and an ethyl group.

In further embodiments the invention relates compounds of formula (I) wherein R⁸ is an ethyl group.

In yet other embodiments the invention relates compounds of formula (I) wherein L is selected from

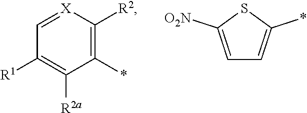

and an ethenylen group.

In other embodiments the invention relates compounds of formula (I) wherein L is selected from

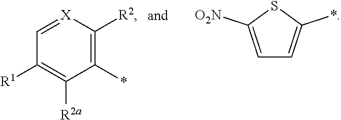

In yet other embodiments the invention relates compounds of formula (I) wherein L is selected from

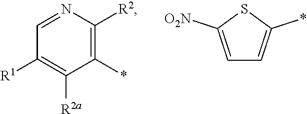

and an ethenylen group.

In yet other embodiments the invention relates compounds of formula (I) wherein L is selected from

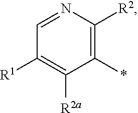

and an ethenylen group.

In yet other embodiments the invention relates compounds of formula (I) wherein L is selected from

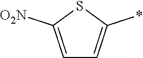 [SMILES: *c1ccc([N+](=O)[O-])s1]

and an ethenylen group.

In yet other embodiments the invention relates compounds of formula (I) wherein L is selected from

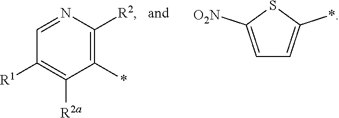

In yet other embodiments the invention relates compounds of formula (I) wherein L is selected from

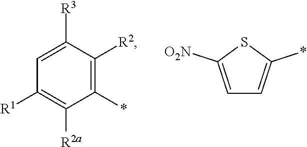

and an ethenylen group.

In yet other embodiments the invention relates compounds of formula (I) wherein L is selected from

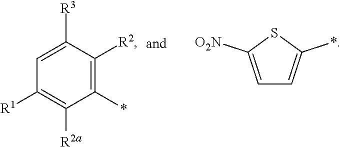

In yet other embodiments the invention relates compounds of formula (I) wherein L is selected from

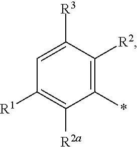

and an ethenylen group.

In yet other embodiments the invention relates compounds of formula (I) wherein L is selected from

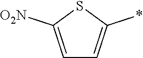 [SMILES: *c1ccc([N+](=O)[O-])s1]

and an ethenylen group.

In yet other embodiments the invention relates compounds of formula (I) wherein L is selected from

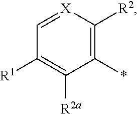

and an ethenylen group.

In yet other embodiments the invention relates compounds of formula (I) wherein L is an ethenylen group.

In yet other embodiments the invention relates compounds of formula (I) wherein L is a

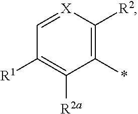

group.

In yet other embodiments the invention relates compounds of formula (I) wherein L is a

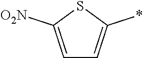 [SMILES: *c1ccc([N+](=O)[O-])s1]

group.

In yet other embodiments the invention relates compounds of formula (I) wherein L is a

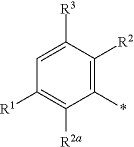

group.

In yet other embodiments the invention relates compounds of formula (I) wherein L is a

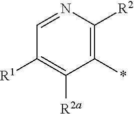

group.

In some embodiments the invention relates to compounds of formula (I), wherein L is an ethenylene group —CH═CH—.

In further embodiments the invention relates compounds of formula (I) wherein Ring A is selected from the groups (a) to (h)

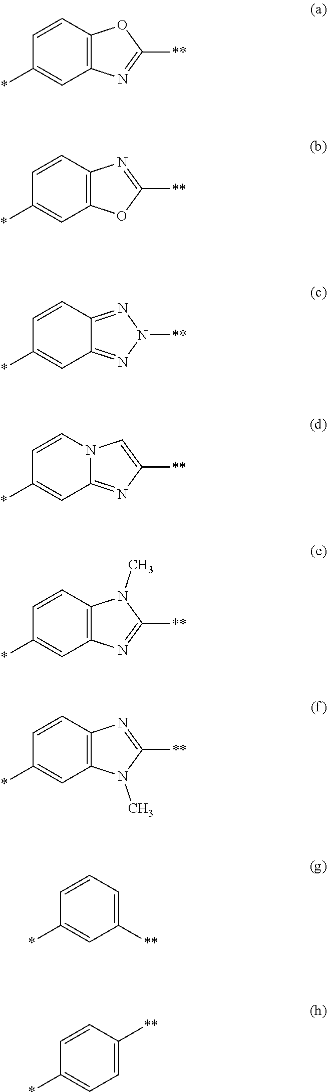

whereby * marks the position where ring A is bound to the nitrogen atom of the amide and ** marks the position where ring A is bound to ring B.

In further embodiments the invention relates compounds of formula (I) wherein Ring A is selected from the groups (c) to (h)

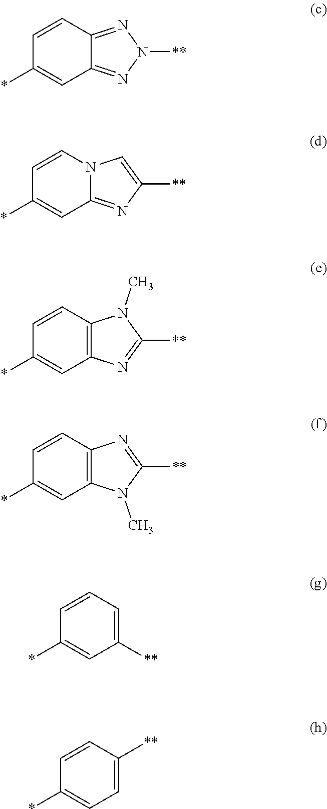

whereby * marks the position where ring A is bound to the nitrogen atom of the amide and ** marks the position where ring A is bound to ring B.

In further embodiments the invention relates compounds of formula (I) wherein Ring A is selected from

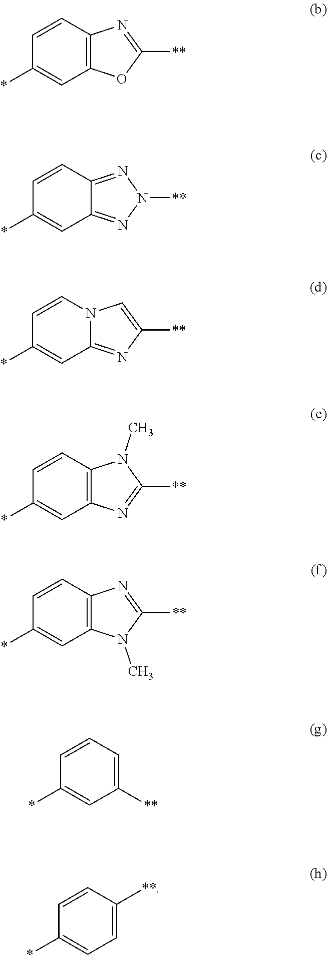

In further embodiments the invention relates compounds of formula (I) wherein Ring A is selected from

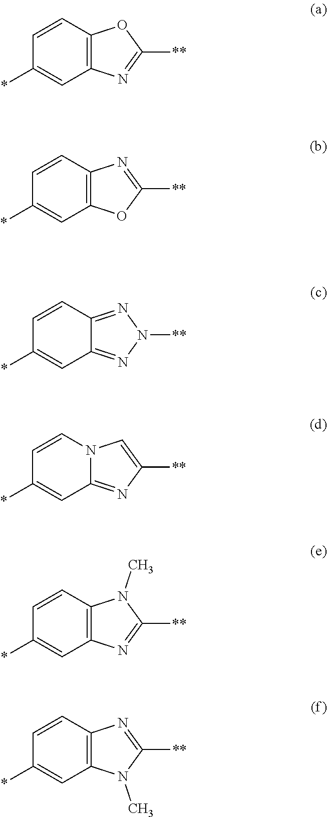

In further embodiments the invention relates compounds of formula (I) wherein Ring A is selected from

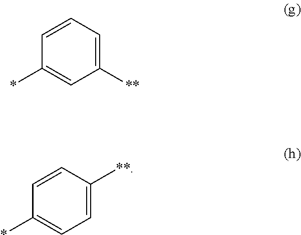

In some embodiments the invention relates compounds of formula (I) wherein Ring B is selected from

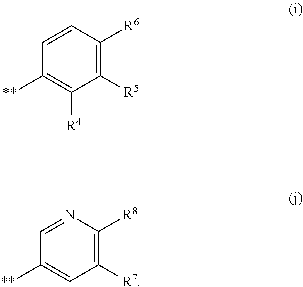

In some embodiments the invention relates compounds of formula (I) wherein Ring B is

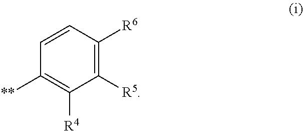

In some embodiments the invention relates compounds of formula (I) wherein Ring B is

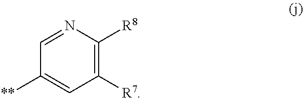

In some embodiments the invention relates compounds of formula (I) wherein Ring B is selected from

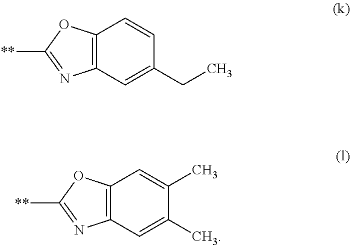

In some embodiments the invention relates compounds of formula (I) wherein Ring B is

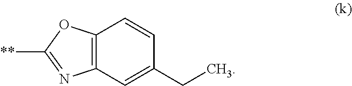

In some embodiments the invention relates compounds of formula (I) wherein Ring B is

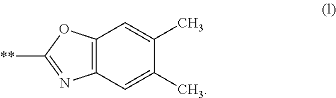

### General Procedures

### Synthetic Routes:

### Route 1

The compounds according to the invention of general formula (I) can be prepared according to the following reflected in schemes 1. The schemes and procedures described below illustrate synthetic routes to the compounds of general formula (I) of the invention and are not intended to be limiting. A person skilled in the art relying on the general knowledge knows that the order of transformations as exemplified in scheme 1 can be modified in various ways. In addition, interconversion of any of the substituents, R¹, R², R³ or R⁴ can be achieved before and/or after the exemplified transformations. These modifications can be such as the introduction of protecting groups, cleavage of protecting groups, reduction or oxidation of functional groups, halogenation, alkylation, acylation, metallation or substitution known to the person skilled in the art. These transformations include those which introduce a functionality which allows for further interconversion of substituents. Appropriate protecting groups and their introduction and cleavage are well-known to the person skilled in the art (see for example T.W. Greene and P.G.M. Wuts in Protective Groups in Organic Synthesis, 3^{rd} edition, Wiley 1999). Specific examples are described in the subsequent paragraphs.

As outlined in scheme 1 below compounds of general formula (I) can be prepared from starting materials of formula (IIa) or (IIb) which are commercially available or are prepared according to procedures disclosed in the experimental section or to common knowledge of the person with ordinary skill by reducing either the nitro group using a reduction agent such as e.g. SnCl₂, preferably with an access of the reagent, more preferably with an access of 4 equivalents, in methanol or ethanol, or transforming the halogenide of formula 2a whereby Hal is selected from bromine and chlorine into the tert.-butylcarbamate under basic conditions using e.g. sodium-tert.butanolate and palladium catalysis with e.g. Pd/charcoal, Pd(dba)₂/tert-Bu XPhos in toluol followed by acidic conditions such as e.g. hydrochloric acid in 1,4-dioxane, more specifically 4M hydrochloric acid, in order to obtain the respective amine of formula (III). Subsequently the so obtained amine is reacted with the compound of formula (IV) or (V) optionally having protected reactive substituents, and optionally by choosing an activation of the acid group of the compounds of formula (IV) or formula (V) using SOCl₂, POCl₃, or other reagents like DCC or DCCI, under addition of an organic base such as e.g. ethylamine, preferably an access of ethylamine, e.g. 4-5 equivalents, in tetrahydrofurane, subsequently optionally deprotecting any protecting group in order to obtain a compound of formula (Ia) having

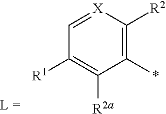

or a compound of formula (Ib) having

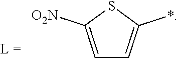

### Step 1:

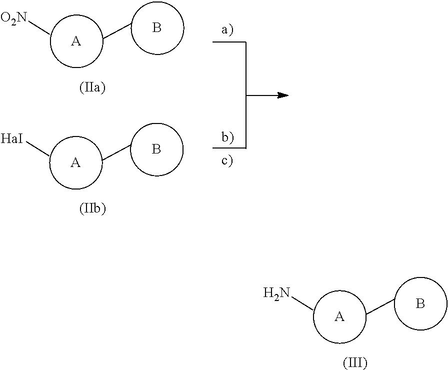

### Step 2:

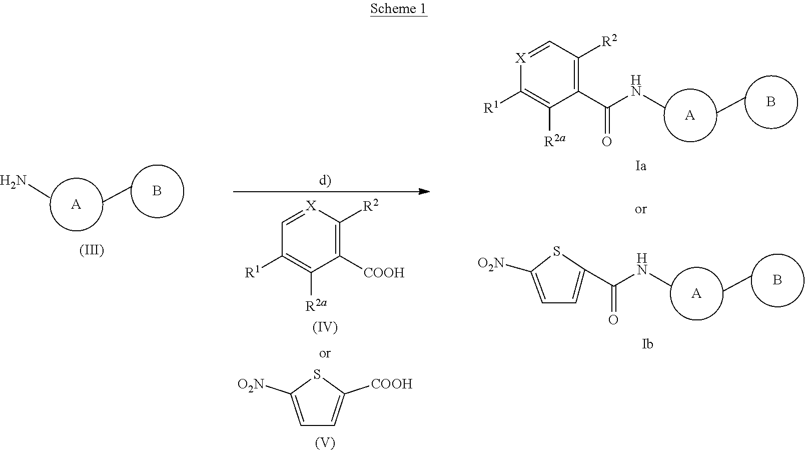

For L=ethenylene please be referred to the conditions used in example 24 and for the sulfonamide please be referred to example 14.

### Pharmaceutical Composition

In accordance with a further aspect, the present invention includes pharmaceutical compositions, in particular a medicament, comprising a compound of general formula (I), as described supra, or a stereoisomer, a tautomer, an N-oxide, a hydrate, a solvate, a salt thereof, particularly a pharmaceutically acceptable salt, or a mixture of same, and one or more excipients), in particular one or more pharmaceutically acceptable excipient(s). Conventional procedures for preparing such pharmaceutical compositions in appropriate dosage forms can be utilized.

The present invention furthermore includes pharmaceutical compositions, in particular medicaments, which comprise at least one compound according to the invention, conventionally together with one or more pharmaceutically suitable excipients, and to their use for the above mentioned purposes.

Furthermore in some embodiments the present invention includes the use of a compound of general formula (I), as described supra, or stereoisomers, tautomers, N-oxides, hydrates, solvates, and salts thereof, particularly pharmaceutically acceptable salts thereof, or mixtures of same, for the preparation of a pharmaceutical composition, preferably a medicament, for the prophylaxis or treatment of diseases, in particular hyperproliferative disorders, particularly cancer disorders.

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropyl-cellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropyl-methylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, polyacrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprise at least one compound according to the invention, conventionally together with one or more pharmaceutically suitable excipient(s), and to their use according to the present invention.

### Combinations

In accordance with another aspect, the present invention includes pharmaceutical combinations, in particular medicaments, comprising at least one compound of general formula (I) of the present invention and at least one or more further active ingredients, in particular for the treatment and/or prophylaxis of a hyperproliferative disorder, more specifically a cancer disorder.

Particularly, the present invention includes a pharmaceutical combination, which comprises:
one or more first active ingredients, in particular compounds of general formula (I) as defined supra, and
one or more further active ingredients, in particular chemotherapeutic agents, more particularly anti-cancer agents.

In one embodiment the compound of the present invention are combined with immune checkpoint inhibitors, which are selected from the group consisting of agents targeting PD1, PD-L1, CTLA4, or immune modulators such as Bacillus Calmette-Guérin (BCG) vaccine.

In an embodiment, the one or more chemotherapeutic agents are selected from the group consisting of an alkylating agent, an anti-metabolite, an anti-microtubule agent, and a topoisomerase inhibitor.

In an embodiment, the one or more chemotherapeutic agents are immune checkpoint inhibitors selected from the group consisting of an agents targeting PD1, PD-L1, CTLA4, or other immune modulators.

In an embodiment, the one or more chemotherapeutic agents are receptor tyrosine kinase inhibitors (RTK). RTK inhibitors are selected from the group consisting of an agents targeting FGFR1, FGFR2, FGFR3, EGFR, ERBB2, or ERBB3.

In an embodiment, the one or more chemotherapeutic agents are Mitogen-activated protein kinase (MAPK) pathway inhibitors selected from the group consisting of an agents targeting KRAS, NRAS, HRAS, BRAF, RAF1 (c-RAF), MAP2K1 (MEK1), MAP2K2 (MEK2), MAPK1 (ERK2), or MAPK3 (ERK1).

The term “combination” in the present invention is used as known to persons skilled in the art, it being possible for said combination to be a fixed combination, a non-fixed combination or a kit-of-parts.

A “fixed combination” in the present invention is used as known to persons skilled in the art and is defined as a combination wherein, for example, a first active ingredient, such as one or more compounds of general formula (I) of the present invention, and a further active ingredient are present together in one unit dosage or in one single entity. One example of a “fixed combination” is a pharmaceutical composition wherein a first active ingredient and a further active ingredient are present in admixture for simultaneous administration, such as in a formulation. Another example of a “fixed combination” is a pharmaceutical combination wherein a first active ingredient and a further active ingredient are present in one unit without being in admixture.

A non-fixed combination or “kit-of-parts” in the present invention is used as known to persons skilled in the art and is defined as a combination wherein a first active ingredient and a further active ingredient are present in more than one unit. One example of a non-fixed combination or kit-of-parts is a combination wherein the first active ingredient and the further active ingredient are present separately. It is possible for the components of the non-fixed combination or kit-of-parts to be administered separately, sequentially, simultaneously, concurrently or chronologically staggered.

The compounds of the present invention can be administered as the sole pharmaceutical agent or in combination with one or more other pharmaceutically active ingredients where the combination causes no unacceptable adverse effects. The present invention also includes such pharmaceutical combinations. For example, the compounds of the present invention can be combined with known anti-cancer.

Examples of anti-cancer agents include:

131I-chTNT, abarelix, abemaciclib, abiraterone, acalabrutinib, aclarubicin, adalimumab, ado-trastuzumab emtansine, afatinib, aflibercept, aldesleukin, alectinib, alemtuzumab, alendronic acid, alitretinoin, alpharadin, altretamine, amifostine, aminoglutethimide, hexyl aminolevulinate, amrubicin, amsacrine, anastrozole, ancestim, anethole dithiolethione, anetumab ravtansine, angiotensin II, antithrombin III, apalutamide, aprepitant, arcitumomab, arglabin, arsenic trioxide, asparaginase, atezolizumab, avelumab, axicabtagene ciloleucel, axitinib, azacitidine, Bacillus Calmette-Guerin (BCG) vaccine, basiliximab, belotecan, bendamustine, besilesomab, belinostat, bevacizumab, bexarotene, bicalutamide, bisantrene, bleomycin, blinatumomab, bortezomib, bosutinib, buserelin, brentuximab vedotin, brigatinib, busulfan, cabazitaxel, cabozantinib, calcitonine, calcium folinate, calcium levofolinate, capecitabine, capromab, carbamazepine carboplatin, carboquone, carfilzomib, carmofur, carmustine, catumaxomab, celecoxib, celmoleukin, cemiplimab, ceritinib, cetuximab, chlorambucil, chlormadinone, chlormethine, cidofovir, cinacalcet, cisplatin, cladribine, clodronic acid, clofarabine, cobimetinib, copanlisib, crisantaspase, crizotinib, cyclophosphamide, cyproterone, cytarabine, dacarbazine, dactinomycin, daratumumab, darbepoetin alfa, dabrafenib, dasatinib, daunorubicin, decitabine, degarelix, denileukin diftitox, denosumab, depreotide, deslorelin, dianhydrogalactitol, dexrazoxane, dibrospidium chloride, dianhydrogalactitol, diclofenac, dinutuximab, docetaxel, dolasetron, doxifluridine, doxorubicin, doxorubicin+estrone, dronabinol, durvalumab, eculizumab, edrecolomab, elliptinium acetate, elotuzumab, eltrombopag, enasidenib, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, ethinylestradiol, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, inotuzumab ozogamicin, interferon alfa, interferon beta, interferon gamma, iobitridol, iobenguane (123I), iomeprol, ipilimumab, irinotecan, Itraconazole, ixabepilone, ixazomib, Ianreotide, Iansoprazole, lapatinib, lasocholine, lenalidomide, lenvatinib, lenograstim, lentinan, letrozole, leuprorelin, levamisole, levonorgestrel, levothyroxine sodium, lisuride, lobaplatin, lomustine, Ionidamine, lutetium Lu 177 dotatate, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, midostaurin, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphinesulfate, mvasi, nabilone, nabiximols, nafarelin, naloxone+pentazocine, naltrexone, nartograstim, necitumumab, nedaplatin, nelarabine, neratinib, neridronic acid, netupitant/palonosetron, nivolumab, pentetreotide, nilotinib, nilutamide, nimorazole, nimotuzumab, nimustine, nintedanib, niraparib, nitracrine, nivolumab, obinutuzumab, octreotide, ofatumumab, olaparib, olaratumab, omacetaxine mepesuccinate, omeprazole, ondansetron, oprelvekin, orgotein, orilotimod, osimertinib, oxaliplatin, oxycodone, oxymetholone, ozogamicine, p53 gene therapy, paclitaxel, palbociclib, palifermin, palladium-103 seed, palonosetron, pamidronic acid, panitumumab, panobinostat, pantoprazole, pazopanib, pegaspargase, PEG-epoetin beta (methoxy PEG-epoetin beta), pembrolizumab, pegfilgrastim, peginterferon alfa-2b, pembrolizumab, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, ribociclib, risedronic acid, rhenium-186 etidronate, rituximab, rolapitant, romidepsin, romiplostim, romurtide, rucaparib, samarium (153Sm) lexidronam, sargramostim, sarilumab, satumomab, secretin, siltuximab, sipuleucel-T, sizofiran, sobuzoxane, sodium glycididazole, sonidegib, sorafenib, stanozolol, streptozocin, sunitinib, talaporfin, talimogene laherparepvec, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tisagenlecleucel, tislelizumab, tocilizumab, topotecan, toremifene, tositumomab, trabectedin, trametinib, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, zorubicin.

### Kits

Also provided herein are kits that can include the compound of formula (I) in form of a therapeutic composition containing an effective amount of said compound in e.g., a unit dosage form.

In some embodiments, the kit comprises a sterile container which includes a therapeutic or diagnostic composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

In particular embodiments, the instructions include at least one of the following: description of the therapeutic agent; dosage schedule and administration for treatment or prevention of malignancy or symptoms thereof; precautions; warnings; indications; counter-indications; over dosage information; adverse reactions; animal pharmacology; clinical studies; and/or references. The instructions may be printed directly on the container (when present), or as a label applied to the container, or as a separate sheet, pamphlet, card, or folder supplied in or with the container.

### Methods and Administration

Compounds of the present invention have surprisingly been found to be PPARG inverse agonists and it is possible therefore that said compounds be used for the treatment or prophylaxis of diseases, preferably hyperproliferative disorders in humans, especially cancer in humans and animals.

Compounds of the present invention can be utilized to inhibit, block, reduce, decrease, etc., cell proliferation and/or cell division. This method comprises administering to a mammal in need thereof, including a human, an amount of a compound of general formula (I) of the present invention, or a pharmaceutically acceptable salt, isomer, polymorph, metabolite, hydrate, solvate or ester thereof, which is effective to treat the hyperproliferative disorder.

Hyperproliferative disorders include, but are not limited to, for example: psoriasis, keloids, and other hyperplasias affecting the skin, benign prostate hyperplasia (BPH), solid tumours, such as cancers of the brain, breast, digestive tract, eye, head and neck, liver, respiratory tract, reproductive organs, skin, thyroid, parathyroid, urinary tract, and their distant metastases. Those disorders also include leukaemias, lymphomas, and sarcomas.

Examples of breast cancers include, but are not limited to, invasive ductal carcinoma, invasive lobular carcinoma, ductal carcinoma in situ, and lobular carcinoma in situ.

Examples of brain cancers include, but are not limited to, brain stem and hypophtalmic glioma, cerebellar and cerebral astrocytoma, medulloblastoma, ependymoma, as well as neuroectodermal and pineal tumour.

Tumours of the digestive tract include, but are not limited to, anal, colon, colorectal, oesophageal, gallbladder, gastric, pancreatic, rectal, small-intestine, and salivary gland cancers.

Eye cancers include, but are not limited to, intraocular melanoma and retinoblastoma.

Head-and-neck cancers include, but are not limited to, laryngeal, hypopharyngeal, nasopharyngeal, oropharyngeal cancer, lip and oral cavity cancer and squamous cell.

Examples of liver cancers include, but are not limited to, hepatocellular carcinoma (liver cell carcinomas with or without fibrolamellar variant), cholangiocarcinoma (intrahepatic bile duct carcinoma), and mixed hepatocellular cholangiocarcinoma.

Examples of cancers of the respiratory tract include, but are not limited to, small-cell and non-small-cell lung carcinoma, as well as bronchial adenoma and pleuropulmonary blastoma.

Tumours of the male reproductive organs include, but are not limited to, prostate and testicular cancer.

Tumours of the female reproductive organs include, but are not limited to, endometrial, cervical, ovarian, vaginal, and vulvar cancer, as well as sarcoma of the uterus.

Tumours of the urinary tract include, but are not limited to, bladder, penile, kidney, renal pelvis, ureter, urethral and human papillary renal cancers.

Bladder cancer includes luminal and non-luminal bladder cancer, basal bladder cancer, muscle-invasive bladder cancer, or non-muscle-invasive bladder cancer.

Skin cancers include, but are not limited to, squamous cell carcinoma, Kaposi's sarcoma, malignant melanoma, Merkel cell skin cancer, and non-melanoma skin cancer.

Leukemias include, but are not limited to, acute myeloid leukemia, acute lymphoblastic leukemia, chronic lymphocytic leukemia, chronic myelogenous leukemia, and hairy cell leukemia.

The hyperproliferative disorders have been well characterized in humans, but also exist with a similar etiology in other mammals, and can be treated by administering pharmaceutical compositions of the present invention.

The “subject” being treated is a human or non-human mammal (e.g., a bovine, a canine, an equine, a feline, an ovine, a primate, and the like).

The term “treating” or “treatment” as stated throughout this document is used conventionally, for example the management or care of a subject for the purpose of combating, alleviating, reducing, relieving, improving the condition of a disease or disorder, such as a carcinoma.

The compounds of the present invention can be used in particular in therapy and prevention, i.e. prophylaxis, of tumour growth and metastases, especially in solid tumours of many indications and stages with or without pre-treatment of the tumour growth.

Thus in some embodiments, the present invention includes a method of inhibiting proliferation of a cell and/or the induction of apoptosis in a cell, comprising contacting the cell with a compound of formula (I) according to any one of claims 1-7.

Particularly in some embodiments, the present invention includes a method of treating a hyperproliferative disorder, more particularly cancer, comprising administering an effective amount of at lest one compound of general formula (I) according to any one of claims 1-7.

A method of inhibiting PPARG activity in a cancer cell is also provided, wherein the method comprises contacting a cancer cell with a compound of general formula (I). The cancer cell may be in vitro or in vivo.

In accordance with a further aspect, the present invention includes a method of treatment or prophylaxis of diseases, in particular hyperproliferative disorders, particularly cancer disorders, using an effective amount of a compound of general formula (I), as described supra, or stereoisomers, tautomers, N-oxides, hydrates, solvates, and salts thereof, particularly pharmaceutically acceptable salts thereof, or mixtures of same.

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., breast cancer, esophageal cancer, pancreatic cancer, colorectal cancer, hepatocellular cancer, and bladder cancer etc.).

In some embodiments, the method of treatment and/or prophylaxis of a hyperproliferative disorder in a subject may comprise administering to the subject an effective amount of a compound of general formula (I). The hyperproliferative disorder may be, for example, cancer (such as e.g., bladder cancer, pancreatic cancer and colon cancer etc.).

Furthermore in some embodiments, the present invention includes a method of treating cancer, particularly breast cancer, esophageal cancer, pancreatic cancer, colorectal cancer, hepatocellular cancer, and bladder cancer comprising administering an effective amount of at least one compound of formula (I) according to any one of claims 1-3.

Furthermore in some embodiments, the present invention includes a method of treating cancer, particularly bladder cancer, pancreatic cancer or colon cancer comprising administering an effective amount of at least one compound of formula (I) according to any one of claims 1-3.

Furthermore in some embodiments, the present invention includes a method of treating cancer, particularly breast cancer, esophageal cancer, pancreatic cancer, colorectal cancer, hepatocellular cancer, and bladder cancer comprising administering an effective amount of at least one compound of formula (I) according to any one of claims 1-3.

Furthermore in some embodiments, the present invention includes a method of treating cancer, particularly bladder cancer, pancreatic cancer and colon cancer comprising administering an effective amount of at least one compound of formula (I) according to any one of claims 1-3.

In accordance with a further aspect, the present invention includes compounds of general formula (I), as described supra, or stereoisomers, tautomers, N-oxides, hydrates, solvates, and salts thereof, particularly pharmaceutically acceptable salts thereof, or mixtures of same, for use in the treatment or prophylaxis of diseases, in particular hyperproliferative disorders, more particularly cancer diseases effecting the following organs: brain, breast, digestive tract, head and neck, liver, respiratory tract, reproductive organs, skin, thyroid, urinary tract, and their distant metastases including leukaemias, lymphomas, and sarcomas.

In some embodiments, the present invention includes a method of using a compound of general formula (I) for the treatment of diseases.

In some embodiments, the present invention includes a compound of general formula (I) for use in a method of inhibiting proliferation of a cell and/or the induction of apoptosis in a cell, comprising contacting the cell with a compound of formula (I) according to any one of claims 1-7.

Particularly in some embodiments, the present invention includes compounds of general formula (I) for use in a method of treating a hyperproliferative disease, more particularly wherein the hyperproliferative disease is cancer, and yet even more particularly wherein the cancer disease is bladder cancer, pancreatic cancer or colon cancer.

In some embodiments the present invention provides for compounds of general formula (I) for use in a method of treating cancer, particularly where the cancer disease is bladder cancer or pancreatic cancer.

In an embodiment, the bladder cancer is luminal and non-luminal bladder cancer, basal bladder cancer, muscle-invasive bladder cancer, or non-muscle-invasive bladder cancer.

Based upon standard laboratory techniques known to evaluate compounds useful for the treatment of hyperproliferative disorders, by standard toxicity tests and by standard pharmacological assays for the determination of treatment of the conditions identified above in mammals, and by comparison of these results with the results of known active ingredients or medicaments that are used to treat these conditions, the effective dosage of the compounds of the present invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of the invention.

### EXPERIMENTAL SECTION

Chemical names were generated using the ACD/Name software from ACD/Labs. In some cases generally accepted names of commercially available reagents were used in place of ACD/Name generated names.

The following table 1 lists the abbreviations used in this paragraph and in the Examples section as far as they are not explained within the text body. Other abbreviations have their meanings customary per se to the skilled person.

TABLE 1
Abbreviations

| Abbreviation | Meaning |
|---|---|
| AcOH | Acetic acid |
| aq. | aqueous |
| ACN | Acetonitrile |
| BSA | Bovine serum albumin |
| br | broad signal (NMR) |
| d | doublet (NMR) |
| DAD | Diode Array Detector |
| DCM | Dichloromethane |
| dd | doublet of doublet (NMR) |
| DIPEA | Diisopropylethylamine |
| DMF | N,N-dimethylformamide |
| DMSO | Dimethylsulfoxide |
| DMEM | Dulbecco's Modified Eagle's Medium |
| DTT | Dithiothreitol |
| EDC•HCl | N-(3-Dimethylaminopropyl)-N′-ethylcarbodiimide hydrochloride salt |
| EDTA | Ethylenediamine tetraacetic acid |
| ESI | Eectrospray (ES) ionization |
| EtOAc | Ethyl acetate |
| EtOH | Ethanol |
| FBS | Fetal bovine serum |
| FRET | Fluorescence resonance energy transfer |
| h, hr (hrs) | hour(s) |
| HATU | (1-[Bis(dimethylamino)methylene]-1H-1,2,3-triazolo[4,5-b]pyridinium |
| | 3-oxide hexafluorophosphate |
| HCl | hydrogen chloride, hydrochloric acid |
| HEPES | 4-(2-hydroxyethyl)-1-piperazineethanesulfonic acid |
| HPLC | High performance liquid chromatography |
| LBD | Ligand binding domain |
| LC-MS | Liquid chromatography-mass spectrometry |
| m | multiplet (NMR) |
| MeCN | Acetonitrile |
| MeOH | Methanol |
| min | minute(s) |
| MS | Mass spectrometry |
| NMR | Nuclear Magnetic Resonance spectroscopy: chemical shifts (δ) |
| | are given in ppm. The chemical shifts were corrected by setting |
| | the DMSO signal to 2.50 ppm using unless otherwise stated. |
| PyBrop | |
| q | quartet (NMR) |
| Rt, rt or r.t. | Room temperature |
| Rₜ or tR or ^{t}R | Retention time |
| s | singulet (NMR) |
| sat. | saturated |
| t | triplet (NMR) |
| td | triplet of doublet (NMR) |
| TEA | triethylamine |
| TFA | Trifluoroacetic acid |
| THF | Tetrahydrofuran |
| T3P | Propanephosphonic acid anhydride |
| δ | Chemical shift |

Other abbreviations have their meanings customary per se to the skilled person.

The various aspects of the invention described in this application are illustrated by the following examples which are not meant to limit the invention in any way.

The example testing experiments described herein serve to illustrate the present invention and the invention is not limited to the examples given.

### Experimental Section—General Part

### General Methods and Materials.

All reagents, for which the synthesis is not described in the experimental part, are either commercially available, or are known compounds or may be formed from known compounds by known methods by a person skilled in the art.

Commercially available reagents and anhydrous solvents were used as supplied, without further purification.

All air- and moisture-sensitive reactions were carried out in oven-dried (at 120° C.) glassware under an inert atmosphere of argon.

### Microwave Method:

A Biotage Initiator Classic microwave reactor was used for reactions conducted in a microwave oven.

### TLC and UPLC Analysis

Reactions were monitored by TLC and UPLC analysis with a Waters Acquity UPLC-MS Single Quad system; column: Acquity UPLC BEH C18 1.7 μm, 50×2.1 mm; basic conditions: eluent A: H₂O+0.2 vol % aq NH₃ (32%), eluent B: MeCN; gradient: 0-1.6 min 1-99% B, 1.6-2.0 min 99% B; flow: 0.8 mL/min; acidic conditions: eluent A: H₂O+0.1 vol % formic acid (99%), eluent B: MeCN; gradient: 0-1.6 min 1-99% B, 1.6-2.0 min 99% B; flow: 0.8 mL/min; temperature: 60° C.; DAD scan: 210-400 nm.

Analytical TLC was carried out on aluminum-backed plates coated with Merck Kieselgel 60 F₂₅₄, with visualization under UV light at 254 nm.

### Flash Chromatography:

Flash chromatography was carried out using a Biotage Isolera One system with 200-400 nm variable detector.

### Preparative HPLC:

Preparative HPLC was carried out with a Waters AutoPurification MS Single Quad system; column: Waters XBridge C18 5 μm, 100×30 mm;
basic conditions: eluent A: H₂O+0.2 vol % aq NH₃ (32%), eluent B: MeCN; gradient: 0-0.5 min 5% B, flow: 25 mL/min; 0.51-5.50 min 10-100% B, flow: 70 mL/min; 5.51-6.5 min 100% B, flow: 70 mL/min;
acidic conditions: eluent A: H₂O+0.1 vol % formic acid (99%), eluent B: MeCN; gradient: 0-0.5 min 5% B, flow: 25 mL/min; 0.51-5.50 min 10-100% B, flow: 70 mL/min; 5.51-6.5 min 100% B, flow: 70 mL/min; temperature: 25° C.; DAD scan: 210-400 nm.

### Low-Resolution Mass Spectra, HPLC-MS:

Low-resolution mass spectra (electrospray ionization, ESI) were obtained via HPLC-MS (ESI) using a Waters Acquity UPLC system equipped with an SQ 3100 Mass Detector; column: Acquity UPLC BEH C18 1.7 μm, 50×2.1 mm; eluent A: H₂O+0.05% formic acid (99%), eluent B: MeCN+0.05% formic acid (99%); gradient: 0-0.5 min 5% B, 0.5-2.5 min 5-100% B, 2.5-4.5 min 100% B; total run time: 5 min; flow: 0.5 mL/min.

### Chiral SFC:

Analysis and separation of mixtures of regioisomers was performed using chiral SFC.

Instrument 1: Agilent: 1260, Aurora SFC-Modul; column: Chiralpak IA 5μ 100×4.6 mm; Eluent A: CO2; Eluent B: Methanol+0.2 Vol-% aqueous ammonia (32%); isocratic: 35% B; flow: 4 ml/min; temperature: 37.5° C.; BPR: 100 bar; UV: 280 nm.

Instrument 2: Sepiatec: Prep SFC100; column: Chiralpak IA 5μ 250×30 mm; eluent A: CO2; eluent B: methanol+0.2 vol % aqueous ammonia (32%); isocratic: 35% B; flow: 100 ml/min; temperature: 40° C.; BPR: 150 bar; UV: 280 nm.

The purity of all target compounds was at least 90%, as determined by UPLC-MS.

Compound names were generated using ICS software.

### NMR Spectra:

The multiplicities of proton signals in ¹H NMR spectra given in the following paragraphs reflect the observed signal form and do not take into account any higher-order signal phenomena. As a rule, the chemical shift data refers to the center of the signal in question. In the case of wide multiplets, a range is specified. Signals hidden by solvent or water were either assigned tentatively or are not listed. Strongly broadened signals—e.g. caused by rapid rotation of molecular moieties or by interchanging protons—have also been assigned tentatively (often referred to as a broad multiplet or broad singlet) or are not shown.

NMR spectra were recorded at rt (22±1° C.), unless otherwise noted, on Bruker Avance III HD spectrometers. ¹H NMR spectra were obtained at 400 or 500 MHz and referenced to the residual solvent signal (7.26 ppm for CDCl₃, 2.50 ppm for DMSO-d₆). ¹H NMR data are reported as follows: chemical shift (δ) in ppm, multiplicity (s=singlet, d=doublet, t=triplet, q=quartet, br=broad, m=multiplet), integration, and assignment.

### Experimental Section—Chemical synthesis

### Synthesis of Intermediate Compounds

### Intermediate 1

### 2-(3-Methylphenyl)-5-nitro-1,3-benzoxazole

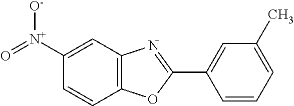 [SMILES: Cc1cccc(-c2nc3cc([N+](=O)[O-])ccc3o2)c1]

2-Amino-4-nitrophenol (1.80 g, 11.7 mmol) was dissolved in 90 toluene, then 1.0 eq. 3-methylbenzoyl chloride (1.5 ml, 12 mmol) was added slowly. This mixture was refluxed for 20 hours. Then 0.25 eq. p-toluenesulfonic acid (555 mg, 2.92 mmol) was added and the mixture was refluxed for 6 more hours. The reaction mixture was allowed to cool to r.t. A dark precipitate had been formed which was collected by filtration and washed with toluene. The filtrate was evaporated under reduced pressure to give 3.54 g of the title compound (83% yield) as crude material which was used directly in the next step.

¹H NMR (400 MHz, DMSO-d₆) δ ppm 2.45 (s, 3H) 7.49-7.58 (m, 2H) 8.01-8.10 (m, 3H) 8.35 (dd, J=8.87, 2.28 Hz, 1H) 8.67 (d, J=2.28 Hz, 1H).

### Intermediate 2

### 2-(3-Methylphenyl)-1,3-benzoxazol-5-amine

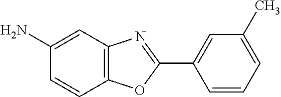 [SMILES: Cc1cccc(-c2nc3cc(N)ccc3o2)c1]

2-(3-Methylphenyl)-5-nitro-1,3-benzoxazole CAS-RN: [103382-61-2] (3.54 g, 13.9 mmol) was dissolved in 130 ml ethanol, then 4.0 eq. tin(II) chloride dihydrate (12.6 g, 55.6 mmol) was added. This mixture was refluxed for 2 hours. The mixture was adjusted with sodium carbonate solution (w=10%) to pH 9 and afterwards extracted 3 times with DCM, washed with water, dried over sodium sulfate, filtered, and evaporated to give 2.48 g (64% yield) crude material of the title compound CAS-RN: [69657-63-2] which was directly used in the next step.

UPLC-MS (acidic conditions): ^{t}R.=0.98 min. MS (ESI+): m/z=225.2 [M+H]+.

### Intermediate 3

### tert.-Butyl [2-(2-methylphenyl)-1,3-benzoxazol-5-yl]carbamate

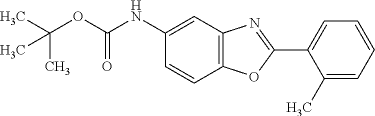 [SMILES: Cc1ccccc1-c1nc2cc(NC(=O)OC(C)(C)C)ccc2o1]

A mixture of 5-bromo-2-(2-methylphenyl)-1,3-benzoxazole (1.02 g, 3.54 mmol), 1.2 eq. tert.-butyl carbamate (498 mg, 4.25 mmol), 0.1 eq. t-Bu-X-Phos (150 mg, 354 μmol), 0.03 eq. Bis(dibenzylidenaceton)palladium(0) (61 mg, 110 μmol) and 2.0 eq. sodium tert.-butylate (680 mg, 7.08 mmol) in 180 ml toluene was stirred at 60° C. for 8 days. Water and DCM were added, the phases were separated, and the organic phase evaporated to dryness to give 1.28 g crude material. Purification by flash chromatography (silica gel, hexane/ethyl acetate gradient) gave 140 mg of the impure title compound (5% yield) alongside 983 mg recovered starting material. The crude material was used without further purification in the next step.

UPLC-MS (acidic conditions): ^{t}R=1.48 min. MS (ESI+): m/z=325.5 [M+H]⁺.

### Intermediate 4

### 2-(2-Methylphenyl)-1,3-benzoxazol-5-amine

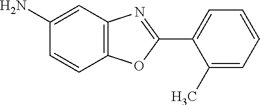 [SMILES: Cc1ccccc1-c1nc2cc(N)ccc2o1]

A mixture of tert.-butyl [2-(2-methylphenyl)-1,3-benzoxazol-5-yl]carbamate (156 mg, 480 μmol, Intermediate 3) and 1.2 ml 4M HCl in dioxane (10 eq., 4.8 mmol) was stirred at r.t. for 18 h. The reaction mixture was evaporated to dryness. To give 155.8 mg (36% yield) of the title compound which was directly used in the next step.

UPLC-MS (acidic conditions): ^{t}R=0.95 min. MS (ESI+): m/z=225.1 [M+H]⁺.

### Intermediate 5

### 5-Bromo-2-(4-ethylphenyl)-1,3-benzoxazole

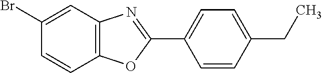 [SMILES: CCc1ccc(-c2nc3cc(Br)ccc3o2)cc1]

4-Ethylbenzoyl chloride (18 ml, 120 mmol) was dissolved in 200 ml toluene and 2.0 eq. sodium bicarbonate dissolved in 200 ml water were added. Under vigorous stirring, 1.0 eq. 2-amino-4-bromophenol (23.4 g, 125 mmol) were added portion-wise, and the mixture stirred over night at r.t. Ethyl acetate was added, and the mixture extracted with water three times. The organic phase was evaporated to dryness and taken up in 200 ml toluene again. 1.0 eq. p-Toluenesulfonic acid (21.4 g, 125 mmol) was added, and the reaction mixture stirred over night at 95° C. After cooling to r.t., MTBE was added, and the reaction mixture washed 3-times with aqueous sodium bicarbonate solution (w=10%). The organic phase was evaporated to dryness and the resulting crude material by flash chromatography (silica gel, hexane/ethyl acetate gradient) to give 23.0 g (61% yield) of the title compound. 1H NMR (400 MHz, DMSO-d6) δ ppm 1.23 (s, 3H) 2.62-2.87 (m, 2H) 7.33-7.50 (m, 2H) 7.51-7.62 (m, 1H) 7.68-7.85 (m, 1H) 7.95-8.05 (m, 1H) 8.05-8.28 (m, 2H).

### Intermediate 6

### tert.-Butyl [2-(4-ethylphenyl)-1,3-benzoxazol-5-yl]carbamate

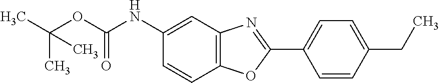 [SMILES: CCc1ccc(-c2nc3cc(NC(=O)OC(C)(C)C)ccc3o2)cc1]

A mixture of 5-bromo-2-(4-ethylphenyl)-1,3-benzoxazole (22.7 g, 75.1 mmol, Intermediate 5) and 1.2 eq. tert.-butyl carbamate (13.2 g, 113 mmol) in toluene was purged with nitrogen gas. 0.1 eq. Bis(dibenzylidenaceton)palladium(0) (4.32 g, 7.5 mmol), 0.3 eq. t-Bu-X-Phos (9.57 g, 22.5 mmol), and 3.0 eq. sodium tert.-butylate (21.7 g, 225 mmol) were added and the reaction mixture stirred at 80° C. for 6 hours. The reaction mixture was evaporated to dryness, water and DCM were added and the phases separated. The combined organic phases were evaporated to dryness again and the resulting crude material purified by flash chromatography (silica gel, hexane/ethyl acetate gradient) to give 19.6 g (77% yield) of the title compound. 1H NMR (400 MHz, DMSO-d6) δ ppm 1.19-1.26 (m, 3H) 1.50 (s, 9H) 2.63-2.77 (m, 2H) 7.38-7.52 (m, 3H) 7.59-7.69 (m, 1H) 7.84-7.97 (m, 1H) 8.03-8.17 (m, 2H) 9.40-9.58 (m, 1H).

### Intermediate 7

### 2-(4-Ethylphenyl)-1,3-benzoxazol-5-amine

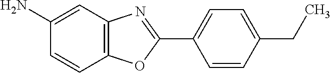 [SMILES: CCc1ccc(-c2nc3cc(N)ccc3o2)cc1]

A mixture of tert.-butyl [2-(4-ethylphenyl)-1,3-benzoxazol-5-yl]carbamate (7.20 g, 21.3 mmol, Intermediate 6) and 53 ml HCl-solution (4 N in dioxane, 10.0 eq.) was stirred at r.t. for 16 h. The reaction was evaporated to dryness. Diluted aqueous NaOH-solution and DCM were added, the phases separated, and the aqueous phase extracted multiple time with DCM. The combined organic phases were again evaporated to dryness to give 4.90 g (97% yield) of the title compound.

UPLC-MS (acidic conditions): ^{t}R=1.10 min. MS (ESI+): m/z=239.3 [M+H]⁺.

### Intermediate 8

### 5-Bromo-2-(3-chlorophenyl)-1,3-benzoxazole

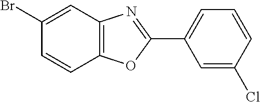 [SMILES: Clc1cccc(-c2nc3cc(Br)ccc3o2)c1]

A mixture of 2-amino-4-bromophenol (2.00 g, 10.6 mmol), 1.2 eq. 3-chlorobenzoyl chloride (2.05 g, 12 mmol) in 40 ml toluene was stirred at 80° C. for 3 days. 1.0 eq. p-toluenesulfonic acid (1.83 g, 10.6 mmol) were added, and the reaction mixture stirred another 3 days at 80° C. The mixture was evaporated to dryness, water and ethyl acetate added and the phases separated. The aqueous phase was extracted multiple times with ethyl acetate, the combined organic phases washed with diluted aqueous sodium hydroxide solution, dried with sodium sulfate and evaporated to dryness again. The remaining crude material was purified by flash chromatography (silica gel, hexane/ethyl acetate gradient) to give 3.0 g (91% yield) of the title compound.

UPLC-MS (acidic conditions): ^{t}R=1.61 min. MS (ESI+): m/z=310.0 [M+H]⁺.

### Intermediate 9

### tert.-Butyl [2-(3-chlorophenyl)-1,3-benzoxazol-5-yl]carbamate

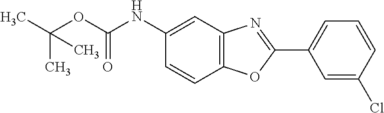 [SMILES: CC(C)(C)OC(=O)Nc1ccc2oc(-c3cccc(Cl)c3)nc2c1]

A mixture of 5-bromo-2-(3-chlorophenyl)-1,3-benzoxazole (3.00 g, 9.72 mmol, Intermediate 8), 2.0 eq. ter.t.-butyl carbamate (2.28 g, 19.4 mmol), 0.1 eq. Bis(dibenzylidenaceton)palladium(0) (0.56 g, 0.97 mmol), 0.1 eq. t-Bu-X-Phos (413 mg, 0.97 mmol) and 2.0 eq. sodium ter.t.-butylate (1.87 g, 19.4 mmol) in 220 ml toluene was stirred at 90° C. for 6 days. The reaction mixture was evaporated to dryness, water and ethyl actetate were added and the phases separated. The aqueous layer was extracted with ethyl acetate. The combined organic phases were washed with saturated sodium chloride solution, dried over sodium sulfate and evaporated to dryness. The resulting crude material was purified by flash chromatography (silica gel, hexane/ethyl acetate gradient) to give 1.66 g (50% yield) of the title compound.

UPLC-MS (acidic conditions): ^{t}R=1.51 min. MS (ESI+): m/z=345.2 [M+H]⁺.

### Intermediate 10

### 2-(3-Chlorophenyl)-1,3-benzoxazol-5-amine

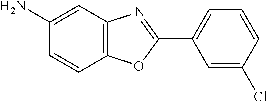 [SMILES: Nc1ccc2oc(-c3cccc(Cl)c3)nc2c1]

A mixture of tert.-butyl [2-(3-chlorophenyl)-1,3-benzoxazol-5-yl]carbamate (1.66 g, 4.81 mmol, Intermediate 9) and 30 ml HCl-solution (4 N in dioxane, 25.0 eq.) was stirred at r.t. for 24 h. The reaction was evaporated to dryness to give the title compound which was used in the next step without further purification.

UPLC-MS (acidic conditions): ^{t}R=1.06 min. MS (ESI+): m/z=246.0 [M+H]⁺.

### Intermediate 11

### 5-Bromo-2-(3-fluoro-4-methoxyphenyl)-1,3-benzoxazole

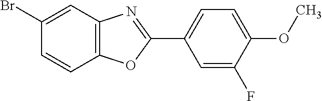 [SMILES: COc1ccc(-c2nc3cc(Br)ccc3o2)cc1F]

A mixture of 3-fluoro-4-methoxybenzoic acid (2.17 g, 12.8 mmol) and 10 eq. SOCl₂ (9.3 ml, 130 mmol) was stirred at 60° C. for 3 h. The mixture was evaporated to dryness and the resulting acid chloride used directly in the next step. A mixture of 2-amino-4-bromophenol (2.39 g, 12.7 mmol), 1.0 eq. 3-fluoro-4-methoxybenzoyl chloride (2.40 g, 12.7 mmol) in 100 ml toluene was stirred at 80° C. for 16 h. 0.6 eq. p-toluenesulfonic acid (1.31 g, 7.64 mmol) were added, and the reaction mixture stirred overnight at 80° C. The mixture was evaporated to dryness, and the remaining crude material was purified by flash chromatography (silica gel, hexane/ethyl acetate gradient) to give 2.13 g (52% yield) of the title compound.

UPLC-MS (acidic conditions): Rₜ=1.48 min. MS (ESI+): m/z=324.0 [M+H]⁺.

### Intermediate 12

### tert.-Butyl [2-(3-fluoro-4-methoxyphenyl)-1,3-benzoxazol-5-yl]carbamate

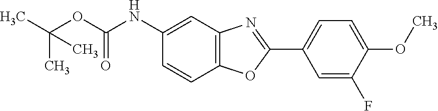 [SMILES: COc1ccc(-c2nc3cc(NC(=O)OC(C)(C)C)ccc3o2)cc1F]

A mixture of 5-bromo-2-(3-fluoro-4-methoxyphenyl)-1,3-benzoxazole (4.00 g, 12.4 mmol, Intermediate 11), 2.0 eq. tert.-butyl carbamate (2.91 g, 24.8 mmol), 0.1 eq. Bis(dibenzylidenaceton)palladium(0) (0.71 g, 1.24 mmol), 0.1 eq. t-Bu-X-Phos (527 mg, 1.24 mmol) and 2.0 eq. sodium ter.t.-butylate (2.39 g, 24.8 mmol) in 280 ml toluene was stirred at 90° C. for 6 days. The reaction mixture was evaporated to dryness, water and ethyl actetate were added and the phases separated. The aqueous layer was extracted with ethyl acetate. The combined organic phases were washed with saturated NaCl-solution, dried over Na₂SO₄ and evaporated to dryness. The resulting crude material was purified by flash chromatography (silica gel, hexane/ethyl acetate gradient) to give 2.14 g (48% yield) of the title compound.

UPLC-MS (acidic conditions): Rₜ=1.37 min. MS (ESI+): m/z=359.4 [M+H]⁺.

### Intermediate 13

### 2-(3-Fluoro-4-methoxyphenyl)-1,3-benzoxazol-5-amine

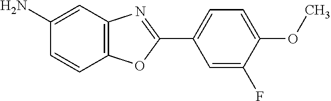 [SMILES: COc1ccc(-c2nc3cc(N)ccc3o2)cc1F]

A mixture of tert.-butyl [2-(3-fluoro-4-methoxyphenyl)-1,3-benzoxazol-5-yl]carbamate (611 mg, 1.70 mmol, Intermediate 12) and 15 ml HCl-solution (4 N in dioxane, 35.0 eq.) was stirred at r.t. until complete conversion. The reaction was evaporated to dryness. 2M aqueous NaOH-solution and DCM were added, the phases separated, and the aqueous phase extracted multiple time with DCM. The combined organic phases were again evaporated to dryness to give 464 mg of the title compound.

LC-MS (acidic conditions): ^{t}R=0.76 min. MS (ESI+): m/z=259.2 [M+H]⁺.

¹H NMR (400 MHz, DMSO-d₆) δ ppm 3.94 (s, 3H) 5.11 (s, 2H) 6.65 (dd, J=8.62, 2.28 Hz, 1H) 6.84 (d, J=2.03 Hz, 1H) 7.34-7.41 (m, 2H) 7.88 (dd, J=12.04, 2.15 Hz, 1H) 7.91-7.97 (m, 1H).

### Intermediate 14

### 5-bromo-2-(5-methylpyridin-3-yl)-1,3-benzoxazole

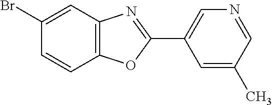 [SMILES: Cc1cncc(-c2nc3cc(Br)ccc3o2)c1]

A mixture of 5-methylpyridine-3-carboxylic acid (3.91 g, 28.5 mmol) and 10 eq. (21 ml, 290 mmol) was stirred at 60° C. for 2 days. The mixture was evaporated to dryness and the resulting acid chloride used directly in the next step. A mixture of 2-amino-4-bromophenol (5.30 g, 28.2 mmol), 1.0 eq. 5-methylpyridine-3-carbonyl chloride (4.39 g, 28.2 mmol) in 110 ml toluene was stirred at 80° C. for 6 days. 0.6 eq. p-toluenesulfonic acid (2.91 g, 16.9 mmol) were added, and the reaction mixture stirred for 3 days at 80° C. The mixture was evaporated to dryness. Water, 31 mL 2N NaOH-solution and DCM were added and the phases separated.

The organic phase was evaporated to dryness and the remaining crude material was purified by flash chromatography (silica gel, hexane/ethyl acetate gradient) to give 200 mg (2.5% yield) of the title compound alongside 2.5 g recovered aminophenol starting material.

UPLC-MS (acidic conditions): ^{t}R=1.27 min. MS (ESI+): m/z=291.0 [M+H]⁺.

### Intermediate 15

### tert.-Butyl [2-(5-methylpyridin-3-yl)-1,3-benzoxazol-5-yl]carbamate

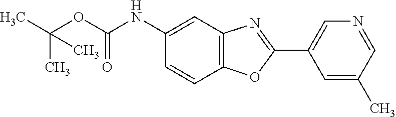 [SMILES: Cc1cncc(-c2nc3cc(NC(=O)OC(C)(C)C)ccc3o2)c1]

A mixture of 5-bromo-2-(5-methylpyridin-3-yl)-1,3-benzoxazole (550 mg, 1.90 mmol, Intermediate 14), 1.2 eq. tert.-butyl carbamate (267 mg, 2.28 mmol), 0.03 eq. Bis(dibenzylidenaceton)palladium(0) (33 mg, 57 μmol), 0.1 eq. t-Bu-X-Phos (80.8 mg, 190 μmol) and 2.0 eq. sodium ter.t.-butylate ((366 mg, 3.80 mmol) in 94 ml toluene was stirred at 90° C. for 4 hours. The reaction mixture was evaporated to dryness, water and DCM were added and the phases separated. The aqueous layer was extracted multiple times with DCM. The combined organic phases were evaporated to dryness. The resulting crude material was purified by flash chromatography (silica gel, hexane/ethyl acetate gradient) to give 500 mg (81% yield) of the title compound.

UPLC-MS (acidic conditions): ^{t}R=1.20 min. MS (ESI+): m/z=326.5 [M+H]⁺.

### Intermediate 16

### 2-(5-Methylpyridin-3-yl)-1,3-benzoxazol-5-amine

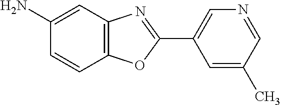 [SMILES: Cc1cncc(-c2nc3cc(N)ccc3o2)c1]

A mixture tert.-butyl [2-(5-methylpyridin-3-yl)-1,3-benzoxazol-5-yl]carbamate (500 mg, 1.54 mmol, Intermediate 15) and 3.8 ml HCl-solution (4 N in dioxane, 10.0 eq.) was stirred at r.t. until complete conversion. Water and DCM were added, and the phases separated. The aqueous phase extracted multiple time with DCM. The combined organic phases were again evaporated to dryness to give 464 mg of the title compound to give 300 mg (87% yield) of the title compound.

UPLC-MS (acidic conditions): ^{t}R=0.66 min. MS (ESI+): m/z=226.2 [M+H]⁺.

Intermediate 16 N-(5-Bromo-2-hydroxyphenyl)-6-ethylpyridine-3-carboxamide

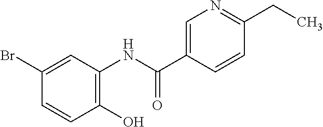 [SMILES: CCc1ccc(C(=O)Nc2cc(Br)ccc2O)cn1]

A mixture of 2-amino-4-bromophenol (2.00 g, 10.6 mmol), 1.2 eq. 6-ethylpyridine-3-carboxylic acid (1.93 g, 12.8 mmol), 1.5 eq. HATU (6.07 g, 16.0 mmol), 5.0 eq. TEA (7.4 ml, 53 mmol) in 50 ml DMF was stirred at r.t. for 2 h. DMF was evaporated under reduced pressure. Water and DCM were added, and the phases separated.

The aqueous layer was extracted multiple times with DCM. The combined organic phases were evaporated to dryness and the remaining crude material purified by flash chromatography (silica gel, hexane/ethyl acetate gradient) to give 1.80 g (53% yield) of the title compound.

LC-MS (acidic conditions): ^{t}R=0.86 min. MS (ESI+): m/z=321.1 [M+H]⁺. ¹H NMR (400 MHz, DMSO-d₆) δ ppm 1.25 (t, J=7.60 Hz, 3H) 2.83 (q, J=7.60 Hz, 2H) 6.88 (d, J=8.62 Hz, 1H) 7.21 (dd, J=8.62, 2.53 Hz, 1H) 7.42 (d, J=8.11 Hz, 1H) 7.88 (d, J=2.28 Hz, 1H) 8.21 (dd, J=8.11, 2.28 Hz, 1H) 9.01 (d, J=1.77 Hz, 1H) 9.70 (s, 1H) 10.13 (s, 1H).

### Intermediate 17

### 5-Bromo-2-(6-ethylpyridin-3-yl)-1,3-benzoxazole

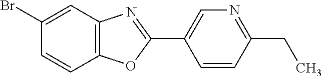 [SMILES: CCc1ccc(-c2nc3cc(Br)ccc3o2)cn1]

A mixture of N-(5-bromo-2-hydroxyphenyl)-6-ethylpyridine-3-carboxamide (1.8 g, 5.60 mmol, Intermediate 16) and 15 ml polyphosphoric acid was stirred at 200° C. for 2 h. After cooling to room temperature, ice water and DCM were added, and the phases separated. The aqueous layer was extracted multiple times with DCM. The combined organic phases were evaporated to dryness to give 1.40 g (82% yield) of the title compound which was used without further purification in the next step.

LC-MS (acidic conditions): ^{t}R=1.27 min. MS (ESI+): m/z=303.1 [M+H]⁺.

¹H NMR (400 MHz, DMSO-d₆) δ ppm 1.28 (t, J=7.60 Hz, 3H) 2.87 (q, J=7.60 Hz, 2H) 7.53 (d, J=7.86 Hz, 1H) 7.61 (dd, J=8.74, 1.90 Hz, 1H) 7.81 (d, J=8.62 Hz, 1H) 8.08 (d, J=2.03 Hz, 1H) 8.43 (dd, J=8.11, 2.28 Hz, 1H) 9.24 (dd, J=2.28, 0.76 Hz, 1H).

### Intermediate 18

### tert.-Butyl [2-(6-ethylpyridin-3-yl)-1,3-benzoxazol-5-yl]carbamate

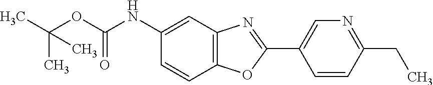 [SMILES: CCc1ccc(-c2nc3cc(NC(=O)OC(C)(C)C)ccc3o2)cn1]

A mixture of 5-bromo-2-(6-ethylpyridin-3-yl)-1,3-benzoxazole (1400 mg, 4.62 mmol, Intermediate 17), 1.5 eq. tert.-butyl carbamate (812 mg, 6.92 mmol), 0.1 eq.

Bis(dibenzylidenaceton)palladium(0) (266 mg, 462 μmol), 0.3 eq. t-Bu-X-Phos (588 mg, 1.39 mmol) and 3.0 eq. sodium ter.t.-butylate (1.33 g, 13.86 mmol) in 38 ml toluene was stirred at 90° C. for 3 hours. The reaction mixture was evaporated to dryness, water and DCM were added and the phases separated. The aqueous layer was extracted multiple times with DCM. The combined organic phases were evaporated to dryness. The resulting crude material was purified by flash chromatography (silica gel, hexane/ethyl acetate gradient) to give 1.30 g (83% yield) of the title compound.

UPLC-MS (acidic conditions): ^{t}R=1.27 min. MS (ESI+): m/z=340.6 [M+H]⁺.

### Intermediate 19

### 2-(6-Ethylpyridin-3-yl)-1,3-benzoxazol-5-amine

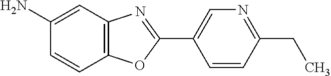 [SMILES: CCc1ccc(-c2nc3cc(N)ccc3o2)cn1]

A mixture tert.-butyl [2-(6-ethylpyridin-3-yl)-1,3-benzoxazol-5-yl]carbamate (1.30 g, 3.83 mmol, Intermediate 18) and 9.6 ml HCl-solution (4 N in dioxane, 10.0 eq.) was stirred at r.t. until complete conversion. Water, saturated NaHCO₃-solution and DCM were added, and the phases separated. The aqueous phase extracted multiple time with DCM. The combined organic phases were again evaporated to dryness to give 464 mg of the title compound to give the title compound which was used without further purification in the next step.

UPLC-MS (acidic conditions): ^{t}R=0.76 min. MS (ESI+): m/z=240.3 [M+H]⁺.

### Intermediate 20

### 5-Bromo-2-(4-ethylphenyl)-1H-benzimidazole

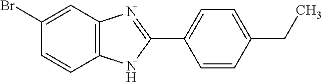 [SMILES: CCc1ccc(-c2nc3cc(Br)ccc3[nH]2)cc1]

A mixture of 4-bromobenzene-1,2-diamine (5.44 g, 29.1 mmol), 1.1 eq. 4-ethylbenzaldehyde (4.29 g, 32.0 mmol), 1.2 eq. oxone (10.7 g, 34.9 mmol) in 2.0 ml water and 100 ml DMF was stirred at r.t. for 17 h. A precipitate had been formed and was filtered of to give 8.0 g (91% yield) of the title compound which was used without further purification in the next step.

UPLC-MS (acidic conditions): ^{t}R=1.11 min. MS (ESI+): m/z=302.4 [M+H]⁺.

### Intermediate 21

### 5-Bromo-2-(4-ethylphenyl)-1-methyl-1H-benzimidazole & 6-Bromo-2-(4-ethylphenyl)-1-methyl-1H-benzimidazole

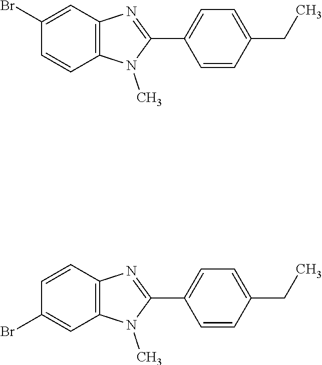 [SMILES: CCc1ccc(-c2nc3cc(Br)ccc3n2C)cc1 ; CCc1ccc(-c2nc3ccc(Br)cc3n2C)cc1]

A mixture of 5-bromo-2-(4-ethylphenyl)-1H-benzimidazole (2.00 g, 6.64 mmol, Intermediate 20), 1.1 eq. iodomethane (455 μl, 7.3 mmol, 1.2 eq. Cs₂CO₃ (2.60 g, 7.97 mmol) in 30 ml acetonitrile was stirred at r.t. until complete conversion. The reaction mixture was filtrated off and evaporated to dryness. The remaining crude material was purified by flash chromatography (silical gel, hexane, ethyl acetate gradient) to give 1.00 g (48% yield) of the title compound as mixture of two regioisomers which were not separated at this step.

UPLC-MS (acidic conditions): ^{t}R=1.20 & 1.21 min. MS (ESI+): m/z=316.4 [M+H]⁺.

LC-MS (acidic conditions): ^{t}R=1.02 min. MS (ESI+): m/z=315.1 [M+H]⁺.

¹H NMR (400 MHz, DMSO-d₆) δ ppm 1.25 (t, J=7.60 Hz, 2×3H) 2.71 (d, J=7.35 Hz, 2×2H) 3.87 (2×s, 2×3H) 3.87-3.90 (m, 1H) 7.37 (dd, J=8.49, 1.90 Hz, 1H) 7.40-7.44 (m, 5H) 7.59-7.63 (m, 2H) 7.75-7.80 (m, 4H) 7.87 (d, J=1.52 Hz, 1H) 7.92 (d, J=2.03 Hz, 1H).

### Intermediate 22

### tert.-Butyl [2-(4-ethylphenyl)-1-methyl-1H-benzimidazol-5-yl]carbamate & tert.-Butyl [2-(4-ethylphenyl)-1-methyl-1H-benzimidazol-6-yl]carbamate

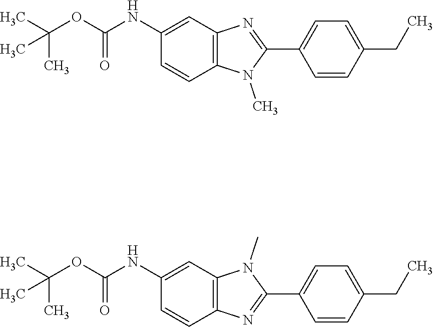

A mixture of 5-bromo-2-(4-ethylphenyl)-1-methyl-1H-benzimidazole and 6-bromo-2-(4-ethylphenyl)-1-methyl-1H-benzimidazole (700 mg, 2.22 mmol, Intermediates 21), 1.2 eq. tert.-butyl carbamate (312 mg, 2.66 mmol), 0.03 eq. Bis(dibenzylidenaceton)palladium(0) (38 mg, 67 μmol), 0.1 eq. t-Bu-X-Phos (94.3 mg, 222 μmol) and 2.0 eq. sodium ter.t.-butylate (427 mg, 4.44 mmol) in 110 ml toluene was stirred at 60° C. for 3 days. The reaction mixture was evaporated to dryness and the resulting crude material was purified by flash chromatography (silica gel, hexane/ethyl acetate gradient) to give 260 g (33% yield) of the title compound as mixture of regioisomers which were not separated at this step.

UPLC-MS (acidic conditions): ^{t}R=1.01 & 1.04 min. MS (ESI+): m/z=352.7 [M+H]⁺.

### Intermediate 23

### 2-(4-Ethylphenyl)-1-methyl-1H-benzimidazol-5-amine & 2-(4-Ethylphenyl)-1-methyl-1H-benzimidazol-6-amine

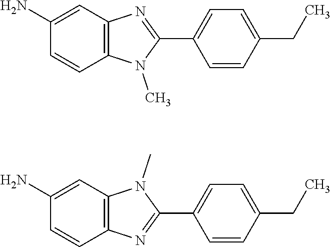 [SMILES: CCc1ccc(-c2nc3cc(N)ccc3n2C)cc1 ; CCc1ccc(-c2nc3ccc(N)cc3n2C)cc1]

A mixture tert.-butyl [2-(4-ethylphenyl)-1-methyl-1H-benzimidazol-5-yl]carbamate & tert.-butyl [2-(4-ethylphenyl)-1-methyl-1H-benzimidazol-6-yl]carbamate (260 mg, 740 μmol, Intermediates 22) and 1.8 ml HCl-solution (4 N in dioxane, 10.0 eq.) was stirred at r.t. until complete conversion. The reaction mixture was evaporated to dryness to give 190 mg of the title compound to give the title compound as mixture of regioisomers which were not separated at this step.

UPLC-MS (acidic conditions): ^{t}R=0.75 & 0.77 min. MS (ESI+): m/z=252.2 [M+H]⁺.

### Intermediate 24

### 2-Phenylimidazo[1,2-a]pyridin-6-amine

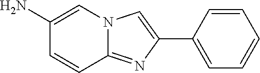 [SMILES: Nc1ccc2nc(-c3ccccc3)cn2c1]

A mixture of 2-(2-chlorophenyl)-6-nitroimidazo[1,2-a]pyridine (5.38 g, 19.7 mmol) made in analogy to the synthesis of 6-Nitro-2-phenylimidazo[1,2-a]pyridine described in RCS Advances (2022), 12(10), 5919-27), compound 3ai and Pd/charcoal in 120 ml MeOH was stirred under H₂-atmosphere at r.t. for 6 hours. The reaction mixture was filtrated over celite and evaporated to dryness to give 2.0 g (49% yield) of the title compound which was used without further purification in the next step.

UPLC-MS (acidic conditions): ^{t}R=0.59 min. MS (ESI+): m/z=210.4 [M+H]⁺.

### Intermediate 25

### 4-(5,6-Dimethyl-1,3-benzoxazol-2-yl)aniline

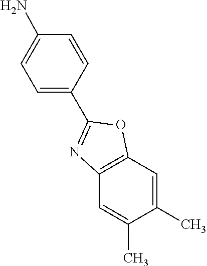 [SMILES: Cc1cc2nc(-c3ccc(N)cc3)oc2cc1C]

Polyphoshoric acid (33 ml, 290 mmol) was heated 180° C. and 1.0 g 4-aminobenzoic acid (7.29 mmol) were added under vigorous stirring. The resulting mixture was stirred at 180° C. for 10 min and 1.0 g 2-amino-4,5-dimethylphenol (7.29 mmol) were added in portions. The resulting mixture was stirred at 180° C. for an additional 2 hours. The mixture was added to ice water. KOH was added and the pH-value adjust to pH: 10. The aqueous phase was extracted multiple times with DCM. The combined organic phases were evaporated to dryness to give 1.1 g crude material of the title compound which was used in the next step without further purification.

UPLC-MS (acidic conditions): R.=1.23 min. MS (ESI+): m/z=239.4 [M+H]⁺.

### Intermediate 26

### tert.-Butyl 2-chloro-5-(methylcarbamoyl)benzoate

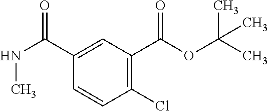 [SMILES: CNC(=O)c1ccc(Cl)c(C(=O)OC(C)(C)C)c1]

A mixture of 3-(ter.t.-butoxycarbonyl)-4-chlorobenzoic acid CAS RN: [862112-39-8] (1.23 g, 4.79 mmol), 2.0 eq. methylamine (4.8 ml, 2.0 M, 9.6 mmol), 2.5 eq. DIPEA (2.1 ml, 12 mmol) and 3.0 eq. HATU (5.47 g, 14.4 mmol) in 289 ml DMF was stirred at r.t. until complete conversion. Water and DCM were added, and the phases separated. The aqueous phase was extracted multiple times with DCM. The combined organic phases were evaporated to dryness and the remaining crude material purified by flash chromatography (silice gel, hexane/ethyl acetate gradient) to give 900 mg (70% yield) of the title compound.

UPLC-MS (acidic conditions): ^{t}R=1.11 min. MS (ESI+): m/z=270.1 [M+H]⁺.

### Intermediate 27

### 2-Chloro-5-(methylcarbamoyl)benzoic acid

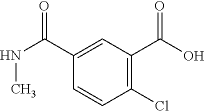

A mixture of tert.-butyl 2-chloro-5-(methylcarbamoyl)benzoate (900 mg, 3.34 mmol, Intermediate 26) and 8.3 ml HCl-solution (4N in dioxane, 10 eq.) was stirred at r.t. until complete conversion. DCM and water were added, and the phases separated. The aqueous phase was extracted multiple times with DCM. The aqueous phase was evaporated to dryness to give 700 mg (98% yield) of the title compound.

UPLC-MS (acidic conditions): ^{t}R=0.59 min. MS (ESI+): m/z=214.0 [M+H]⁺.

### Synthesis of Example Compounds

### Example 1

### 2-Chloro-N-[2-(3-methylphenyl)-1,3-benzoxazol-5-yl]-5-nitrobenzamide

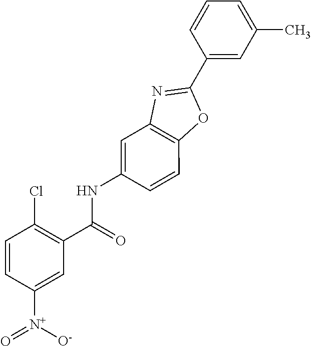 [SMILES: Cc1cccc(-c2nc3cc(NC(=O)c4cc([N+](=O)[O-])ccc4Cl)ccc3o2)c1]

2-(3-Methylphenyl)-1,3-benzoxazol-5-amine (200 mg, 892 μmol, Intermediate 2), 1.2 eq. 2-chloro-5-nitrobenzoic acid (216 mg, 1.07 mmol) and 1.3 eq. propanephosphonic acid anhydride (w=50% in DMF) were dissolved in 12 ml DMF under argon atmosphere. The mixture was stirred at r.t. for 18h. DMF was evaporated under reduced pressure. Aqueous NaHCO₃-solution and ethyl acetate were added to the mixture and the layers were separated. The aqueous layer was extracted multiple times with ethyl acetate. The combined organic layers were washed with saturated NaCl-solution, dried over sodium sulfate, filtered, and evaporated to give the title compound as crude material. Purification by flash chromatography (silica gel, hexane/ethyl acetate gradient) gave 187 mg raw material. This material was suspended in DCM/MeOH (9:1), filtered and washed again with DCM/MeOH (9:1) to give 31.8 mg (9% yield) of the title compound 6a.

LC-MS (acidic conditions): ^{t}R=1.34 min. MS (ESI+): m/z=408.1 [M+H]⁺.

¹H NMR (400 MHz, DMSO-d₆) δ ppm 2.44 (s, 3H) 7.44-7.55 (m, 2H) 7.66 (dd, J=8.87, 2.03 Hz, 1H) 7.80 (d, J=8.87 Hz, 1H) 7.92 (d, J=8.87 Hz, 1H) 7.98-8.03 (m, 1H) 8.05 (s, 1H) 8.24 (d, J=1.77 Hz, 1H) 8.36 (dd, J=8.87, 2.79 Hz, 1H) 8.53 (d, J=2.79 Hz, 1H) 10.91 (s, 1H).

### Example 2

### 2-Chloro-N-[2-(2-methylphenyl)-1,3-benzoxazol-5-yl]-5-nitrobenzamide

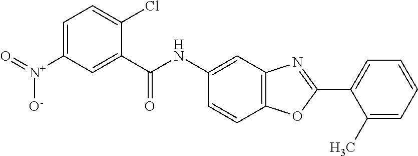 [SMILES: Cc1ccccc1-c1nc2cc(NC(=O)c3cc([N+](=O)[O-])ccc3Cl)ccc2o1]

A mixture of 2-chloro-5-nitrobenzoic acid (145 mg, 719 μmol) and 20 eq. thionyl chloride (1.0 ml, 14 mmol) was stirred at 80° C. for 2.5 hours. The reaction mixture was evaporated to dryness and immediately used in the next step. A mixture of 2-(2-methylphenyl)-1,3-benzoxazol-5-amine (156 mg, 695 μmol, Intermediate 4), 1 eq. 2-chloro-5-nitrobenzoyl chloride (153 mg, 695 μmol) and 5.0 eq. triethylamine (480 μl, 3.5 mmol) in 3.2 ml THF was stirred at r.t. for 4 days. The mixture was evaporated to dryness and the remaining material purified by preparative HPLC (acidic conditions) to give 21.0 mg (7% yield) of the title compound.

LC-MS (acidic conditions): ^{t}R=1.34 min. MS (ESI+): m/z=408.2 [M+H]⁺.

¹H NMR (400 MHz, DMSO-d₆) δ ppm 2.77 (s, 3H) 7.41-7.56 (m, 3H) 7.66 (dd, J=8.87, 2.03 Hz, 1H) 7.81 (d, J=8.87 Hz, 1H) 7.92 (d, J=8.87 Hz, 1H) 8.15 (dd, J=7.86, 1.27 Hz, 1H) 8.27 (d, J=2.03 Hz, 1H) 8.37 (dd, J=8.87, 2.79 Hz, 1H) 8.53 (d, J=2.79 Hz, 1H) 10.91 (s, 1H).

### Example 3

### 2-Chloro-N-[2-(4-ethylphenyl)-1,3-benzoxazol-5-yl]-5-nitrobenzamide

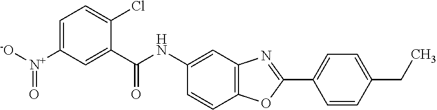

A mixture of 2-(4-ethylphenyl)-1,3-benzoxazol-5-amine (250 mg, 1.05 mmol, Intermediate 7), 1.5 eq. 2-chloro-5-nitrobenzoic acid (317 mg, 1.57 mmol), 2 eq. HATU (798 mg, 2.10 mmol), 5.0 eq. DIPEA (910 μl, 5.2 mmol) in 4.6 ml DMF was stirred at r.t. ° C. for 12 h. Water and saturated sodium bicarbonate solution were added, the mixture extracted with ethyl acetate, washed with brine, dried via sodium sulfate and evaporated to dryness. The remaining crude material was purified by flash chromatography (silica gel, hexane/ethyl acetate gradient) to give 190 mg (43% yield) of the title compound.

LC-MS (acidic conditions): ^{t}R=1.42 min. MS (ESI+): m/z=422.2 [M+H]⁺.

¹H NMR (400 MHz, DMSO-d₆) δ ppm 1.24 (t, J=7.60 Hz, 3H) 2.72 (q, J=7.60 Hz, 2H) 7.47 (d, J=8.62 Hz, 2H) 7.65 (dd, J=8.87, 2.03 Hz, 1H) 7.79 (d, J=8.87 Hz, 1H) 7.92 (d, J=8.87 Hz, 1H) 8.09-8.16 (m, 2H) 8.22 (d, J=1.77 Hz, 1H) 8.36 (dd, J=8.87, 2.79 Hz, 1H) 8.53 (d, J=2.79 Hz, 1H) 10.90 (s, 1H).

### Example 4

### 2-Chloro-N-[2-(3-chlorophenyl)-1,3-benzoxazol-5-yl]-5-nitrobenzamide

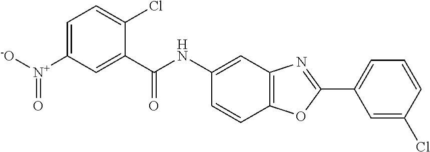

A mixture of 2-(3-chlorophenyl)-1,3-benzoxazol-5-amine (100 mg, 409 μmol, Intermediate 10), 1.4 eq. 2-chloro-5-nitrobenzoic acid (115 mg, 572 μmol), 1.3 eq. HATU (202 mg, 531 μmol), 3.0 eq. DIPEA (210 μl, 1.2 mmol) in 1.8 ml DMF was stirred at r.t. until complete conversion. Water and ethyl acetate were added, and the phases separated. The aqueous phase was extracted with multiple times with ethyl acetate, washed with saturated sodium chloride solution, dried with sodium sulfate and evaporated to dryness. The remaining crude material was purified by preparative HPLC (acidic conditions) to give 29 mg (16% yield) of the title compound.

LC-MS (acidic conditions): ^{t}R=1.37 min. MS (ESI+): m/z=428.0 [M+H]⁺.

¹H NMR (400 MHz, DMSO-d₆) δ ppm 7.64-7.76 (m, 3H) 7.83 (d, J=8.87 Hz, 1H) 7.92 (d, J=8.87 Hz, 1H) 8.15-8.21 (m, 2H) 8.27 (d, J=1.77 Hz, 1H) 8.37 (dd, J=8.87, 2.79 Hz, 1H) 8.53 (d, J=2.79 Hz, 1H) 10.94 (s, 1H).

### Example 5

### N-[2-(2-Bromophenyl)-1,3-benzoxazol-5-yl]-2-chloro-5-nitrobenzamide

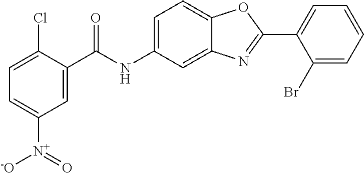

Commercially available, CAS-RN: [312717-21-8]

UPLC-MS (acidic conditions): Rₜ=1.34 min. MS (ESI+): m/z=474.2 [M+H]⁺.

¹H NMR (400 MHz, DMSO-d₆) δ ppm 7.54-7.66 (m, 2H) 7.70 (dd, J=8.87, 2.03 Hz, 1H) 7.85 (d, J=8.87 Hz, 1H) 7.89-7.95 (m, 2H) 8.10 (dd, J=7.73, 1.65 Hz, 1H) 8.30 (d, J=1.77 Hz, 1H) 8.37 (dd, J=8.62, 2.79 Hz, 1H) 8.54 (d, J=2.53 Hz, 1H) 10.95 (s, 1H).

### Example 6

### 2-Chloro-N-[2-(3-fluoro-4-methoxyphenyl)-1,3-benzoxazol-5-yl]-5-nitrobenzamide

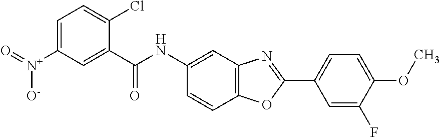 [SMILES: COc1ccc(-c2nc3cc(NC(=O)c4cc([N+](=O)[O-])ccc4Cl)ccc3o2)cc1F]

A mixture of 2-(3-fluoro-4-methoxyphenyl)-1,3-benzoxazol-5-amine (92.0 mg, 356 μmol, Intermediate 13), 1.3 eq. 2-chloro-5-nitrobenzoic acid (93.3 mg, 463 μmol), 2.0 eq. PyBrop (332 mg, 712 μmol), 5.0 eq. DIPEA (310 μl, 1.8 mmol) in 2.9 ml DMF was stirred at r.t. for 3d. Water and DCM were added, the phases separated, and the aqueous phase extracted multiple times with DCM. The combined organic phases were evaporated to dryness. The remaining crude material was purified by preparative HPLC (basic conditions) to give 50 mg (32% yield) of the title compound.

LC-MS (acidic conditions): ^{t}R=1.27 min. MS (ESI+): m/z=442.2 [M+H]⁺.

¹H NMR (400 MHz, DMSO-d₆) δ ppm 3.96 (s, 3H) 7.42 (t, J=8.74 Hz, 1H) 7.65 (dd, J=8.87, 2.03 Hz, 1H) 7.78 (d, J=8.87 Hz, 1H) 7.92 (d, J=8.87 Hz, 1H) 7.95-8.05 (m, 2H) 8.21 (d, J=1.77 Hz, 1H) 8.36 (dd, J=8.62, 2.79 Hz, 1H) 8.53 (d, J=2.79 Hz, 1H) 10.90 (s, 1H).

### Example 7

### 2-Chloro-N-[2-(5-methylpyridin-3-yl)-1,3-benzoxazol-5-yl]-5-nitrobenzamide

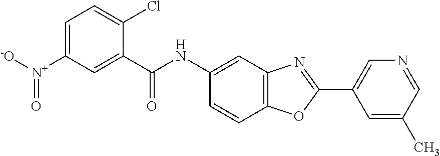

A mixture of 2-(5-methylpyridin-3-yl)-1,3-benzoxazol-5-amine (41.0 mg, 182 μmol, Intermediate 17), 1.3 eq. 2-chloro-5-nitrobenzoic acid (47.7 mg, 237 μmol), 2.0 eq. PyBrop (170 mg, 364 μmol), 4.0 eq. DIPEA (130 μl, 730 μmol) and 0.05 eq. 4-dimethylaminopyridine (1.11 mg, 9.10 μmol) in 0.8 ml DMF was stirred at r.t. until complete conversion. The reaction mixture was evaporated to dryness. The remaining crude material was purified by flash chromatography (silica gel, hexane/ethyl actetate gradient) to give 30 mg (36% yield) of the title compound. LC-MS (acidic conditions): ^{t}R=1.05 min. MS (ESI+): m/z=409.1 [M+H]⁺.

¹H NMR (400 MHz, DMSO-d₆) δ ppm 2.45 (s, 3H) 7.69 (dd, J=8.87, 2.03 Hz, 1H) 7.84 (d, J=8.87 Hz, 1H) 7.92 (d, J=8.87 Hz, 1H) 8.28 (d, J=1.77 Hz, 1H) 8.33-8.42 (m, 2H) 8.54 (d, J=2.79 Hz, 1H) 8.66 (d, J=1.27 Hz, 1H) 9.17 (d, J=1.77 Hz, 1H) 10.94 (s, 1H).

### Example 8

### 2-Chloro-N-[2-(6-ethylpyridin-3-yl)-1,3-benzoxazol-5-yl]-5-nitrobenzamide

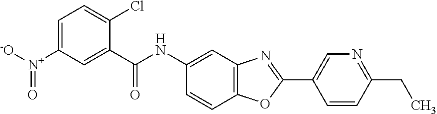

A mixture of 2-(6-ethylpyridin-3-yl)-1,3-benzoxazol-5-amine (88.0 mg, 368 μmol, Intermediate 19), 1.1 eq. 2-chloro-5-nitrobenzoic acid (81.5 mg, 405 μmol), 5.0 eq. TEA (260 μl, 1.8 mmol), 2.0 eq. HATU (280 mg, 0.74 mmol) in 4.4 ml DMF was stirred at r.t. until complete conversion. The reaction mixture was evaporated to dryness and purified by preparative HPLC (acidic conditions) to give 50.0 mg (29% yield) of the title compound.

LC-MS (acidic conditions): ^{t}R=1.14 min. MS (ESI+): m/z=423.1 [M+H]⁺.

¹H NMR (400 MHz, DMSO-d₆) δ ppm 1.29 (t, J=7.60 Hz, 3H) 2.88 (q, J=7.60 Hz, 2H) 7.54 (d, J=8.36 Hz, 1H) 7.69 (dd, J=8.87, 2.03 Hz, 1H) 7.83 (d, J=8.87 Hz, 1H) 7.92 (d, J=8.62 Hz, 1H) 8.26 (d, J=1.77 Hz, 1H) 8.36 (dd, J=8.87, 2.79 Hz, 1H) 8.45 (dd, J=8.24, 2.41 Hz, 1H) 8.53 (d, J=2.79 Hz, 1H) 9.23-9.29 (m, 1H) 10.93 (s, 1H).

### Example 9

### 2-Chloro-N-[2-(4-methylphenyl)-1,3-benzoxazol-6-yl]-5-nitrobenzamide

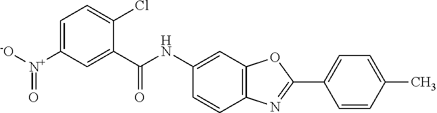

A mixture of 2-(4-methylphenyl)-1,3-benzoxazol-6-amine CAS RN: [69657-63-2] (80.0 mg, 357 μmol,), 1.3 eq. 2-chloro-5-nitrobenzoic acid (93.5 mg, 464 μmol), 2.0 eq. PyBrop (333 mg, 713 μmol) and 5.0 eq. DIPEA (310 μl, 1.8 mmol) in 2.7 ml DMF was stirred at r.t. until complete conversion. The reaction mixture was evaporated to dryness. The remaining crude material was purified by preparative HPLC (acidic conditions) to give 38 mg (25% yield) of the title compound.

LC-MS (acidic conditions): ^{t}R=1.36 min. MS (ESI+): m/z=407.9 [M+H]⁺.

¹H NMR (400 MHz, DMSO-d₆) δ ppm 2.42 (s, 3H) 7.44 (d, J=8.11 Hz, 2H) 7.56 (dd, J=8.74, 1.90 Hz, 1H) 7.79 (d, J=8.87 Hz, 1H) 7.92 (d, J=8.87 Hz, 1H) 8.10 (d, J=8.11 Hz, 2H) 8.32 (d, J=1.77 Hz, 1H) 8.37 (dd, J=8.87, 2.79 Hz, 1H) 8.53 (d, J=2.79 Hz, 1H) 11.02 (s, 1H).

### Example 10

### 2-Chloro-N-[2-(4-ethylphenyl)-1-methyl-1H-benzimidazol-5-yl]-5-nitrobenzamide

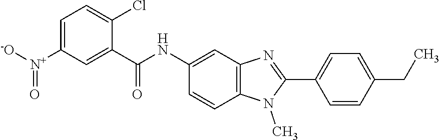

A mixture of 2-chloro-5-nitrobenzoic acid (200 mg, 992 μmol) and 20 eq. thionyl chloride (1.4 ml, 20 mmol) was stirred at 80° C. for 2 hours. The mixture was evaporated to dryness and used immediately in the next step. A mixture of 2-(4-ethylphenyl)-1-methyl-1H-benzimidazol-5-amine and 2-(4-ethylphenyl)-1-methyl-1H-benzimidazol-6-amine (190 mg, 756 μmol), 1.3 eq. 2-chloro-5-nitrobenzoyl chloride (216 mg, 983 μmol), 3.0 eq. TEA (320 μl, 2.3 mmol) in 3.5 ml THF was stirred at r.t. until complete conversion. DCM and water were added, and the phases separated.

The aqueous phase was extracted multiple times with DCM. The combined organic phases were evaporated to dryness and the remaining crude material purified by flash chromatography to give 290 mg of a mixture of regioisomers which were separated by chiral preparative SFC. Separation of regioisomers gave 90 mg (27% yield) of the regioisomer indicated above.

Analytical SFC: ^{t}R=4.58 min.

Preparative SFC: ^{t}R=14.7-19.3 min (99% purity).

LC-MS (acidic conditions): ^{t}R=0.90 min. MS (ESI+): m/z=435.2 [M+H]⁺.

¹H NMR (500 MHz, DMSO-d₆) δ ppm 1.26 (t, J=7.63 Hz, 3H) 2.72 (q, J=7.63 Hz, 2H) 3.89 (s, 3H) 7.43 (d, J=8.27 Hz, 2H) 7.54-7.62 (m, 2H) 7.79 (d, J=8.27 Hz, 2H) 7.91 (d, J=8.90 Hz, 1H) 8.11 (d, J=1.59 Hz, 1H) 8.33-8.38 (m, 1H) 8.50 (d, J=2.86 Hz, 1H) 10.71 (s, 1H).

### Example 11

### 2-Chloro-N-[2-(4-ethylphenyl)-1-methyl-1H-benzimidazol-6-yl]-5-nitrobenzamide

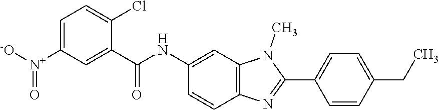

Separation of regioisomers gave 128 mg (39% yield) of the other regioisomer obtained in example 10.

Analytical SFC: ^{t}R=2.98 min.

Preparative SFC: ^{t}R=9.8-12.8 min (98% purity).

LC-MS (acidic conditions): ^{t}R=0.94 min. MS (ESI+): m/z=435.2 [M+H]⁺.

¹H NMR (500 MHz, DMSO-d₆) δ ppm 1.26 (t, J=7.63 Hz, 3H) 2.72 (q, J=7.63 Hz, 2H) 3.86 (s, 3H) 7.37 (dd, J=8.58, 1.91 Hz, 1H) 7.42 (d, J=8.27 Hz, 2H) 7.66 (d, J=8.58 Hz, 1H) 7.75-7.79 (m, 2H) 7.91 (d, J=8.58 Hz, 1H) 8.18 (d, J=1.91 Hz, 1H) 8.36 (dd, J=8.90, 2.86 Hz, 1H) 8.49 (d, J=2.86 Hz, 1H) 10.83 (s, 1H).

### Example 12

### 2-Chloro-5-nitro-N-(2-phenylimidazo[1,2-a]pyridin-6-yl)benzamide

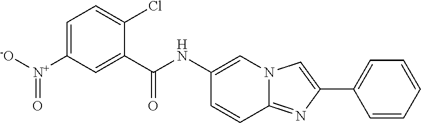

A mixture of 2-phenylimidazo[1,2-a]pyridin-6-amine (100 mg, 478 μmol, Intermediate 24), 1.3 eq. 2-chloro-5-nitrobenzoic acid (125 mg, 621 μmol), 2.0 eq. PyBrop (446 mg, 956 μmol) and 5.0 eq. DIPEA (420 μl, 2.4 mmol) in 3.9 ml DMF was stirred at r.t. until complete conversion. Water and DCM were added, and the phases separated. The aqueous phase was extracted multiple times with DCM. The combined organic phases were evaporated to dryness. The remaining crude material was purified by preparative HPLC (acidic conditions) to give 20 mg 11% yield, 95% purity) of the title compound.

LC-MS (acidic conditions): ^{t}R=0.78 min. MS (ESI+): m/z=393.1 [M+H]⁺.

¹H NMR (400 MHz, DMSO-d₆) δ ppm 7.25-7.35 (m, 2H) 7.41-7.48 (m, 2H) 7.63 (d, J=9.63 Hz, 1H) 7.89-7.98 (m, 3H) 8.37 (dd, J=8.87, 2.79 Hz, 1H) 8.54-8.58 (m, 2H) 9.34 (dd, J=2.03, 1.01 Hz, 1H) 10.91 (s, 1H).

### Example 13

### 2-Chloro-N-[2-(4-methylphenyl)-2H-benzotriazol-5-yl]-5-nitrobenzamide (Xe)

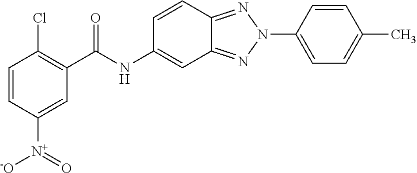

A mixture of 2-(4-methylphenyl)-2H-benzotriazol-5-amine CAS RN: [6659-91-2] (353 mg, 1.57 mmol), 1.5 eq. 2-chloro-5-nitrobenzoic acid (476 mg, 2.36 mmol, 2.0 eq. HATU (1.20 g, 3.15 mmol) and 5.0 eq. DIPEA (1.4 ml, 7.9 mmol) in 6.9 ml DMF was stirred at r.t. until complete conversion. The remaining crude material was purified by flash chromatography (silica gel, hexane/ethyl acetate gradient) to give 227 mg (36% yield, 100% purity) of the title compound.

UPLC-MS (acidic conditions): ^{t}R=1.40 min. MS (ESI+): m/z=408.3 [M+H]⁺.

LC-MS (acidic conditions): ^{t}R=1.40 min. MS (ESI+): m/z=408.1 [M+H]⁺.

¹H NMR (400 MHz, DMSO-d₆) δ ppm 2.42 (s, 3H) 7.46 (d, J=8.36 Hz, 2H) 7.61 (dd, J=9.25, 1.90 Hz, 1H) 7.93 (d, J=8.87 Hz, 1H) 8.05 (d, J=8.62 Hz, 1H) 8.20 (d, J=8.62 Hz, 2H) 8.38 (dd, J=8.87, 2.79 Hz, 1H) 8.57 (d, J=2.53 Hz, 2H) 11.06 (s, 1H).

### Example 14

### 2-Chloro-N-[2-(4-ethylphenyl)-1,3-benzoxazol-5-yl]-5-nitrobenzene-1-sulfonamide

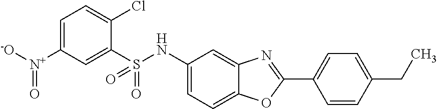

A mixture of 2-(4-ethylphenyl)-1,3-benzoxazol-5-amine (98.0 mg, 411 μmol, Intermediate 7), 1.0 eq. 2-chloro-5-nitrobenzene-1-sulfonyl chloride (105 mg, 411 μmol), 1.1 eq. TEA (63 μl, 450 μmol) in 2.0 ml DCM was stirred at r.t. for 17 h. Water and DCM were added, and the phases separated. The aqueous phase was extracted multiple times with DCM. The combined organic phases were evaporated to dryness. The remaining crude material was purified by preparative HPLC (acidic conditions) to give 95.0 mg (50% yield) of the title compound.

LC-MS (acidic conditions): ^{t}R=1.42 min. MS (ESI+): m/z=458.1 [M+H]⁺.

¹H NMR (400 MHz, DMSO-d₆) δ ppm 1.21 (t, J=7.60 Hz, 3H) 2.69 (q, J=7.60 Hz, 2H) 7.18 (dd, J=8.74, 2.15 Hz, 1H) 7.43 (d, J=8.36 Hz, 2H) 7.49 (d, J=2.28 Hz, 1H) 7.69 (d, J=8.62 Hz, 1H) 7.97 (d, J=8.87 Hz, 1H) 8.01-8.08 (m, 2H) 8.40 (dd, J=8.62, 2.79 Hz, 1H) 8.65 (d, J=2.53 Hz, 1H) 11.06 (s, 1H).

### Example 15

### 2-chloro-N-[4-(5,6-dimethyl-1,3-benzoxazol-2-yl)phenyl]-5-nitrobenzamide

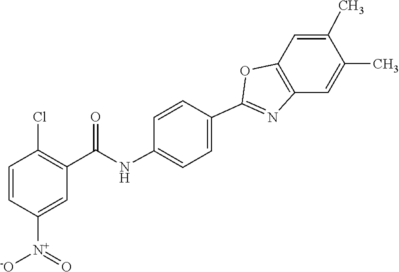

A mixture of 4-(5,6-dimethyl-1,3-benzoxazol-2-yl)aniline (750 mg, 3.15 mmol, Intermediate 25), 1.3 eq. 2-chloro-5-nitrobenzoic acid (825 mg, 4.09 mmol), 1.5 eq. HATU (1.80 g, 4.72 mmol) and 5.0 eq. TEA (2.2 ml, 16 mmol) in 14 ml DMF was stirred at r.t. until complete conversion. DCM and water were added, and the phases separated. The aqueous phase was extracted multiple times with DCM. The combined organic phases were evaporated to dryness and the remaining crude material purified by flash chromatography (silica gel, hexane/ethyl acetate gradient) followed by preparative HPLC (acidic conditions) to give 190 mg (100% purity, 14% yield) of the title compound.

LC-MS (acidic conditions): ^{t}R=1.43 min. MS (ESI+): m/z=422.1 [M+H]⁺.

UPLC-MS (acidic conditions): ^{t}R=1.43 min. MS (ESI+): m/z=422.3 [M+H]⁺.

¹H NMR (400 MHz, DMSO-d₆) δ ppm 2.34 (s, 3H) 2.36 (s, 3H) 7.57 (d, J=4.56 Hz, 2H) 7.90-7.96 (m, 3H) 8.16-8.22 (m, 2H) 8.37 (dd, J=8.74, 2.66 Hz, 1H) 8.55 (d, J=2.79 Hz, 1H) 11.07 (s, 1H).

### Example 16

### 2-Chloro-N-[3-(5-ethyl-1,3-benzoxazol-2-yl)phenyl]-5-nitrobenzamide

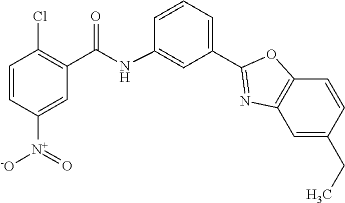

Commercially available, CAS-RN: [420830-10-0]

UPLC-MS (acidic conditions): ^{t}R=1.44 min. MS (ESI+): m/z=422.3 [M+H]⁺.

¹H NMR (400 MHz, DMSO-d₆) δ ppm 1.25 (t, J=7.60 Hz, 3H) 2.71-2.79 (m, 2H) 7.30 (dd, J=8.49, 1.65 Hz, 1H) 7.59-7.68 (m, 2H) 7.72 (d, J=8.36 Hz, 1H) 7.80-7.86 (m, 1H) 7.93 (d, J=8.87 Hz, 1H) 7.95-8.00 (m, 1H) 8.37 (dd, J=8.87, 2.79 Hz, 1H) 8.56 (d, J=2.53 Hz, 1H) 8.74 (t, J=1.77 Hz, 1H) 11.02 (s, 1H).

### Example 17

### 2-Chloro-N-[2-(4-ethylphenyl)-1,3-benzoxazol-5-yl]-5-(trifluoromethyl)benzamide

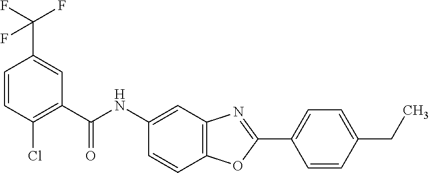 [SMILES: CCc1ccc(-c2nc3cc(NC(=O)c4cc(C(F)(F)F)ccc4Cl)ccc3o2)cc1]

A mixture of 2-(4-ethylphenyl)-1,3-benzoxazol-5-amine (80.0 mg, 336 μmol, Intermediate 7), 1.3 eq. 2-chloro-5-(trifluoromethyl)benzoic acid (98.0 mg, 436 μmol), 0.05 eq. DMAP (2.05 mg, 16.8 μmol), 4.0 eq. DIPEA (230 μl, 1.3 mmol) and 2.0 eq. PyBrop (313 mg, 671 μmol) in 1.5 ml DMF was stirred at r.t. until complete conversion. The reaction mixture was evaporated to dryness and the remaining crude material purified by preparative HPLC (acidic conditions) to give 45.0 mg (30% yield) of the title compound.

LC-MS (acidic conditions): ^{t}R=1.51 min. MS (ESI+): m/z=445.2 [M+H]⁺.

¹H NMR (400 MHz, DMSO-d₆) δ ppm 1.24 (t, J=7.60 Hz, 3H) 2.72 (d, J=7.60 Hz, 2H) 7.47 (d, J=8.62 Hz, 2H) 7.65 (dd, J=8.74, 2.15 Hz, 1H) 7.78 (d, J=8.62 Hz, 1H) 7.83-7.89 (m, 1H) 7.89-7.94 (m, 1H) 8.09 (d, J=2.28 Hz, 1H) 8.11-8.15 (m, 2H) 8.23 (d, J=1.77 Hz, 1H) 10.77-10.88 (m, 1H).

### Example 18

### 4-Chloro-N³-[2-(4-ethylphenyl)-1,3-benzoxazol-5-yl]-N¹-methylbenzene-1,3-dicarboxamide

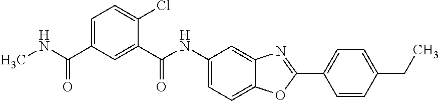 [SMILES: CCc1ccc(-c2nc3cc(NC(=O)c4cc(C(=O)NC)ccc4Cl)ccc3o2)cc1]

A mixture of 2-chloro-5-(methylcarbamoyl)benzoic acid (300 mg, 1.40 mmol, Intermediate 27) and 20 eq. thionyl chloride (2.0 ml, 28 mmol) was stirred at 80° C. for 2.5 hours. The mixture was evaporated to dryness and used immediately in the next step. A mixture of 2-(4-ethylphenyl)-1,3-benzoxazol-5-amine (156 mg, 657 μmol) and 1.05 eq. 2-chloro-5-(methylcarbamoyl)benzoyl chloride (160 mg, 689 μmol) and 3.0 eq. TEA (270 μl, 2.0 mmol) in 4.0 ml THF at r.t. for 12 hours and evaporated to dryness. The crude material was taken up in DCM, stirred at r.t., filtered and evaporated to dryness again to give 150 mg (52% yield) of the title compound.

LC-MS (acidic conditions): ^{t}R=1.24 min. MS (ESI+): m/z=434.1 [M+H]⁺.

¹H NMR (400 MHz, DMSO-d₆) δ ppm 1.23 (t, J=7.60 Hz, 3H) 2.72 (d, J=7.60 Hz, 2H) 2.79 (d, J=4.56 Hz, 3H) 7.47 (d, J=8.36 Hz, 2H) 7.66-7.74 (m, 2H) 7.75-7.80 (m, 1H) 7.99 (dd, J=8.36, 2.28 Hz, 1H) 8.09-8.16 (m, 3H) 8.27 (d, J=1.77 Hz, 1H) 8.82 (br d, J=4.82 Hz, 1H) 10.87 (s, 1H).

### Example 19

### 2-Chloro-N-[2-(4-ethylphenyl)-1,3-benzoxazol-5-yl]pyridine-3-carboxamide

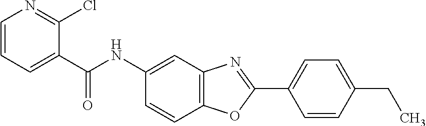 [SMILES: CCc1ccc(-c2nc3cc(NC(=O)c4cccnc4Cl)ccc3o2)cc1]

A mixture of 2-(4-ethylphenyl)-1,3-benzoxazol-5-amine (100 mg, 420 μmol, Intermediate 7), 1.0 eq. 2-chloropyridine-3-carboxylic acid (66.1 mg, 420 μmol), 5.0 eq. TEA (290 μl, 2.1 mmol) and 2.0 eq. HATU (319 mg, 839 μmol) in 6.0 ml THF was stirred at r.t. until complete conversion and evaporated to dryness. Water and DCM were added, and the phases separated. The organic phase was evaporated to dryness and the remaining crude material purified by preparative HPLC (acidic conditions) to give 14 mg (9% yield) of the title compound.

LC-MS (acidic conditions): ^{t}R=1.27 min. MS (ESI+): m/z=378.2 [M+H]⁺.

¹H NMR (400 MHz, DMSO-d₆) δ ppm 1.24 (t, J=7.60 Hz, 3H) 2.68-2.76 (m, 2H) 7.47 (d, J=8.62 Hz, 2H) 7.59 (dd, J=7.60, 4.82 Hz, 1H) 7.65 (dd, J=8.87, 2.03 Hz, 1H) 7.78 (d, J=8.87 Hz, 1H) 8.10-8.16 (m, 3H) 8.22 (d, J=2.03 Hz, 1H) 8.56 (dd, J=4.82, 1.77 Hz, 1H) 10.78-10.91 (m, 1H).

### Example 20

### 2-chloro-5-cyano-N-[2-(4-ethylphenyl)-1,3-benzoxazol-5-yl]benzamide

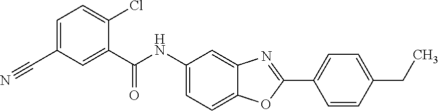 [SMILES: CCc1ccc(-c2nc3cc(NC(=O)c4cc(C#N)ccc4Cl)ccc3o2)cc1]

A mixture of 2-(4-ethylphenyl)-1,3-benzoxazol-5-amine (100 mg, 420 μmol, Intermediate 7), 1.5 eq. 2-chloro-5-cyanobenzoic acid (114 mg, 629 μmol, 5.0 eq. DIPEA (370 μl, 2.1 mmol) and 1.5 eq. HATU (239 mg, 629 μmol) in 2.4 ml DMF was stirred at r.t. until complete conversion and evaporated to dryness. The remaining crude material purified by preparative HPLC (acidic conditions) to give 68 mg (38% yield) of the title compound.

LC-MS (acidic conditions): ^{t}R=1.38 min. MS (ESI+): m/z=402.2 [M+H]⁺.

¹H NMR (400 MHz, METHANOL-d₄) δ ppm 1.29 (t, J=7.60 Hz, 3H) 2.76 (q, J=7.60 Hz, 2H) 7.43 (d, J=8.62 Hz, 2H) 7.59-7.64 (m, 1H) 7.65-7.69 (m, 1H) 7.74 (d, J=8.36 Hz, 1H) 7.85 (dd, J=8.36, 2.03 Hz, 1H) 8.03 (d, J=2.03 Hz, 1H) 8.13-8.18 (m, 2H) 8.21 (d, J=1.52 Hz, 1H).

### Example 21

### N-[2-(4-Ethylphenyl)-1,3-benzoxazol-5-yl]-3-nitrobenzamide

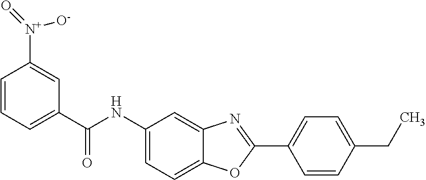 [SMILES: CCc1ccc(-c2nc3cc(NC(=O)c4cccc([N+](=O)[O-])c4)ccc3o2)cc1]

A mixture of 2-(4-ethylphenyl)-1,3-benzoxazol-5-amine (90.0 mg, 378 μmol, Intermediate 7), 1.3 eq. 3-nitrobenzoic acid (82.1 mg, 491 μmol), 2 eq. PyBrop (352 mg, 755 μmol) and 5.0 eq. DIPEA (330 μl, 1.9 mmol) in 3.1 ml DMF was stirred at r.t. until complete conversion. The mixture was extracted multiple times with DCM. The combined organic phases were washed with water and evaporated to dryness. The remaining crude material purified by preparative HPLC (acidic conditions) to give 20 mg (14% yield) of the title compound.

LC-MS (acidic conditions): ^{t}R=1.39 min. MS (ESI+): m/z=388.2 [M+H]⁺.

¹H NMR (400 MHz, DMSO-d₆) δ ppm 1.24 (t, J=7.60 Hz, 3H) 2.68-2.77 (m, 2H) 7.47 (d, J=8.62 Hz, 2H) 7.72-7.82 (m, 2H) 7.87 (t, J=7.98 Hz, 1H) 8.09-8.16 (m, 2H) 8.27 (d, J=1.77 Hz, 1H) 8.42-8.49 (m, 2H) 8.83 (t, J=2.03 Hz, 1H) 10.77 (s, 1H).

### Example 22

### 2-Chloro-N-[2-(3-methylphenyl)-1,3-benzoxazol-5-yl]-3-nitrobenzamide

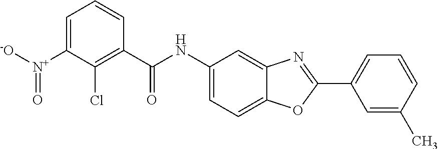

A mixture of 2-(3-methylphenyl)-1,3-benzoxazol-5-amine (99.0 mg, 441 μmol, Intermediate 2), 1.2 eq. 2-chloro-3-nitrobenzoic acid (107 mg, 530 μmol), 5.0 eq. TEA (310 μl, 2.2 mmol) and 1.2 eq. HATU (201 mg, 530 μmol) in 2.0 ml DMF was stirred at r.t. until complete conversion and evaporated to dryness. The remaining crude material purified by flash chromatography (silica gel, hexane/ethyl acetate gradient) to give 83 mg (46% yield) of the title compound.

UPLC-MS (basic conditions): ^{t}R=1.34 min. MS (ESI+): m/z=408.2 [M+H]⁺.

¹H NMR (400 MHz, DMSO-d₆) δ ppm 2.44 (s, 3H) 7.44-7.55 (m, 2H) 7.66 (dd, J=8.74, 2.15 Hz, 1H) 7.72-7.83 (m, 2H) 7.95-8.07 (m, 3H) 8.18 (dd, J=8.11, 1.52 Hz, 1H) 8.24 (d, J=1.77 Hz, 1H) 10.91 (s, 1H),

### Example 23

### N-[2-(4-Ethylphenyl)-1,3-benzoxazol-5-yl]-5-nitrothiophene-2-carboxamide

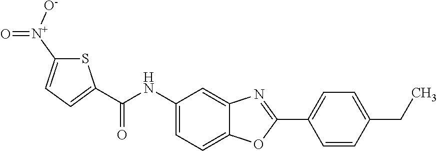 [SMILES: CCc1ccc(-c2nc3cc(NC(=O)c4ccc([N+](=O)[O-])s4)ccc3o2)cc1]

A mixture of 2-(4-ethylphenyl)-1,3-benzoxazol-5-amine (90.0 mg, 378 μmol, Intermediate 7), 1.3 eq. 5-nitrothiophene-2-carboxylic acid (85.0 mg, 491 μmol), 2 eq. PyBrop (352 mg, 755 μmol) and 5.0 eq. DIPEA (330 μl, 1.9 mmol) in 3.1 ml DMF was stirred at r.t. until complete conversion. DCM and water were added, and the phases separated. The aqueous phase was extracted multiple times with DCM. The combined organic phases were evaporated to dryness. Methanol was added to the remaining crude material and the solid material filtered off to give 15 mg (10% yield) of the title compound.

LC-MS (acidic conditions): ^{t}R=1.42 min. MS (ESI+): m/z=394.1 [M+H]⁺.

¹H NMR (400 MHz, DMSO-d₆) δ ppm 1.24 (t, J=7.60 Hz, 3H) 2.72 (q, J=7.60 Hz, 2H) 7.47 (d, J=8.36 Hz, 2H) 7.69 (dd, J=8.87, 2.03 Hz, 1H) 7.80 (d, J=8.87 Hz, 1H) 8.07-8.15 (m, 3H) 8.20 (d, J=2.03 Hz, 1H) 8.24 (d, J=4.31 Hz, 1H) 10.81 (s, 1H).

### Example 24

### N-[2-(4-Ethylphenyl)-1,3-benzoxazol-5-yl]prop-2-enamide

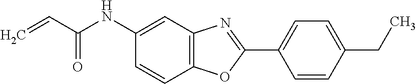 [SMILES: C=CC(=O)Nc1ccc2oc(-c3ccc(CC)cc3)nc2c1]

A mixture of 2-(4-ethylphenyl)-1,3-benzoxazol-5-amine (99.0 mg, 415 μmol, Intermediate 7), 1.3 eq. prop-2-enoic acid (38.9 mg, 540 μmol), 4.0 eq. DIPEA (290 μl, 1.7 mmol) and 1.2 eq. HATU (190 mg, 499 μmol) in 1.8 ml DMF was stirred at r.t. until complete conversion. DCM and water were added, and the phases separated. The aqueous phase was extracted multiple times with DCM. The combined organic phases were evaporated to dryness. The remaining crude material purified by preparative HPLC (acidic conditions) to give 26 mg (20% yield) of the title compound LC-MS (acidic conditions): ^{t}R=1.21 min. MS (ESI+): m/z=293.1 [M+H]⁺.

¹H NMR (400 MHz, DMSO-d₆) δ ppm 1.21-1.26 (m, 3H) 2.72 (q, J=7.60 Hz, 2H) 5.79 (dd, J=9.89, 2.03 Hz, 1H) 6.26-6.34 (m, 1H) 6.41-6.52 (m, 1H) 7.46 (d, J=8.36 Hz, 2H) 7.58 (dd, J=8.87, 2.03 Hz, 1H) 7.73 (d, J=8.62 Hz, 1H) 8.09-8.14 (m, 2H) 8.23 (d, J=2.03 Hz, 1H) 10.34 (s, 1H)

### Example 25

### N-[2-(4-Ethylphenyl)-1,3-benzoxazol-5-yl]-2-fluoro-5-nitrobenzamide

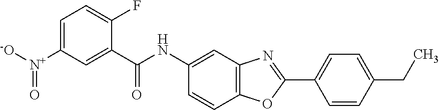

A mixture of 2-(4-ethylphenyl)-1,3-benzoxazol-5-amine (80.0 mg, 336 μmol, Intermediate 7), 1.3 eq. 2-fluoro-5-nitrobenzoic acid (80.8 mg, 436 μmol), 2 eq. PyBrop (313 mg, 671 μmol), 0.05 eq.

DMAP (2.05 mg, 16.8 μmol) and 4.0 eq. DIPEA (230 μl, 1.3 mmol) in 1.5 ml DMF was stirred at r.t. until complete conversion. DCM and water were added, and the phases separated. The aqueous phase was extracted multiple times with DCM. The combined organic phases were evaporated to dryness. The remaining crude material was purified by preparative HPLC (acidic conditions) to give 30 mg (22% yield) of the title compound.

LC-MS (acidic conditions): ^{t}R=1.40 min. MS (ESI+): m/z=406.2 [M+H]⁺.

¹H NMR (400 MHz, DMSO-d₆) δ ppm 1.24 (t, J=7.60 Hz, 3H) 2.68-2.76 (m, 2H) 7.47 (d, J=8.36 Hz, 2H) 7.63-7.75 (m, 2H) 7.79 (d, J=8.62 Hz, 1H) 8.10-8.16 (m, 2H) 8.23 (d, J=2.03 Hz, 1H) 8.45-8.52 (m, 1H) 8.59 (dd, J=5.96, 2.91 Hz, 1H) 10.70-10.95 (m, 1H).

### Example 26

### 2-Bromo-N-[2-(4-ethylphenyl)-1,3-benzoxazol-5-yl]-5-nitrobenzamide

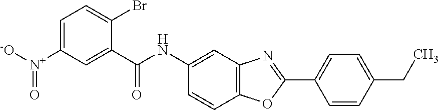

A mixture of 2-(4-ethylphenyl)-1,3-benzoxazol-5-amine (200 mg, 839 μmol, Intermediate 7), 1.05 eq. 2-bromo-5-nitrobenzoic acid (217 mg, 881 μmol), 2 eq. PyBrop (783 mg, 1.68 mmol), 0.05 eq. DMAP (5.13 mg, 42.0 μmol) and 4.0 eq. DIPEA (580 μl, 3.4 mmol) in 3.7 ml DMF was stirred at r.t. until complete conversion. DCM and water were added, and the phases separated. The aqueous phase was extracted multiple times with ethyl acetate. The combined organic phases were evaporated to dryness. The remaining crude material was purified by preparative HPLC (acidic conditions) to give 65 mg (16% yield) of the title compound.

LC-MS (acidic conditions): ^{t}R=1.43 min. MS (ESI+): m/z=468.2 [M+H]⁺.

¹H NMR (400 MHz, DMSO-d₆) δ ppm 1.24 (t, J=7.60 Hz, 3H) 2.72 (q, J=7.60 Hz, 2H) 7.47 (d, J=8.62 Hz, 2H) 7.65 (dd, J=8.74, 2.15 Hz, 1H) 7.79 (d, J=8.87 Hz, 1H) 8.07 (d, J=8.87 Hz, 1H) 8.11-8.16 (m, 2H) 8.22 (d, J=2.03 Hz, 1H) 8.26 (dd, J=8.87, 2.79 Hz, 1H) 8.47 (d, J=2.53 Hz, 1H) 10.88 (s, 1H).

### Example 27

### N-[2-(4-ethylphenyl)-1,3-benzoxazol-5-yl]-2-iodo-5-nitrobenzamide

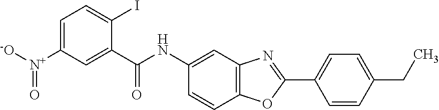

A mixture of 2-(4-ethylphenyl)-1,3-benzoxazol-5-amine (70.0 mg, 294 μmol), 1.2 eq2-iodo-5-nitrobenzoic acid (103 mg, 353 μmol), 2.4 eq. DIPEA (120 μl, 710 μmol) and 1.2 eq. HATU (134 mg, 353 μmol) in 2.7 ml DMF was stirred at r.t. until complete conversion. The reaction mixture was evaporated to dryness and the remaining crude material purified by preparative HPLC (acidic conditions) to give 37 mg (24% yield, 10% purity) of the title compound.

LC-MS (acidic conditions): ^{t}R=1.42 min. MS (ESI+): m/z=514.0 [M+H]⁺.

¹H NMR (400 MHz, DMSO-d₆) δ ppm 1.24 (t, J=7.60 Hz, 3H) 2.69-2.77 (m, 2H) 7.48 (d, J=8.62 Hz, 2H) 7.66 (dd, J=8.87, 2.03 Hz, 1H) 7.80 (d, J=8.87 Hz, 1H) 8.04 (dd, J=8.62, 2.79 Hz, 1H) 8.13 (d, J=8.11 Hz, 2H) 8.21 (d, J=1.77 Hz, 1H) 8.28 (d, J=8.62 Hz, 1H) 8.34 (d, J=2.79 Hz, 1H) 10.81 (s, 1H).

### Experimental Section—Biological Assays

The pharmacological activity of the compounds according to the invention can be assessed using in vitro- and/or in vivo-assays, as known to the person skilled in the art. The following examples describe the biological activity of the compounds according to the invention.

Example compounds according to the invention were tested in selected biological assays one or more times. When tested more than once, data are reported as either average values or as median values, wherein
the average value, also referred to as the arithmetic mean value, represents the sum of the values obtained divided by the number of times tested, and
the median value represents the middle number of the group of values when ranked in ascending or descending order. If the number of values in the data set is odd, the median is the middle value. If the number of values in the data set is even, the median is the arithmetic mean of the two middle values.

Examples were synthesized one or more times. When synthesized more than once, data from biological assays represent average values or median values calculated utilizing data sets obtained from testing of one or more synthetic batches.

The in vitro activity of the compounds of the present invention can be demonstrated in the following assays:

### Cellular In Vitro Assay for Determining Inverse Agonist Activity of Compounds

The identification and quantification of inverse agonism on the PPARG receptors was carried out using the bladder cancer cell line RT-112/84. The gene of NanoLuc Luciferase was engineered into the 3′-UTR of FABP4 in RT-112/84 cells using Cas9-guided homology dependent repair, leading to PPARG dependent expression of the NanoLuc® Luciferase (Goldstein, J. T.; Berger, A. C.; Shih, J.; Duke, F. F.; Furst, L.; Kwiatkowski, D. J.; Cherniack, A. D.; Meyerson, M.; Strathdee, C. A., Genomic Activation of PPARG Reveals a Candidate Therapeutic Axis in Bladder Cancer. Cancer Res 2017, 77 (24), 6987-6998). Inverse agonists reduce the basal expression of the Luciferase resulting in a decrease of the Luciferase signal after addition of substrate and cell lysis. To exclude toxic effects of the compounds a parallel measurement of cell viability was performed based on the CellTiter Glo Assay (Promega).

### Test Procedure:

5000 cells per well were plated out in 30 μl culture medium (DMEM/F12 (PAN Biotech), 10% FBS (Sigma), 2 mM glutamine (Gibco), 10 mM HEPES (Gibco)) in 384-well microtiter plates and kept in a cell incubator (96% humidity, 5% v/v CO2, 37° C.). On the next day 10 μl of test compounds dissolved in DMEM/F12 2% FBS and 0.01% BSA are placed in the wells of the microtiter plates. Final concentrations ranged from 50 μM to 0.03 nM. Plates were incubated over night in a cell incubator (96% humidity, 5% v/v CO2, 37° C.). On the next day the NanoLuc Luciferase activity was measured by addition of 40 μl of NanoLuc substrate and lysis buffer (Promega, Madison, Wisconsin) diluted in 2 mM Ca-tyrode buffer (20 mM HEPES, 130 mM NaCl, 5 mM KCl, 5 mM NaHCO₃, 2 mM MgCl2, pH 7.4). The resulting light signal was measured for 60 seconds in a luminometer. Every sample was tested in quadruples and mean values were used to calculate based on a Marquardt-Levenberg-algorithm the IC50 values and efficacies of the compounds. A model of nonlinear regression curve fit was used for calculation.

TABLE 1

| | PPARG RT- | PPARG_RT- |
|---|---|---|
| | 112_FABP4_NLuc_inverse | 112_FABP4_NLuc_inverse |
| Example | agonist | agonist |
| No. | IC50 - [mol/l] | Efficacy - [%] |
| 1 | 1.30E−6 | 87 |
| 2 | 1.95E−7 | 52 |
| 3 | 1.70e−10 | 94 |
| 4 | 5.15E−8 | 93 |
| 5 | 4.80E−7 | 133 |
| 6 | 2.30E−10 | 82 |
| 7 | 1.60E−10 | 72 |
| 8 | 2.50E−10 | 107 |
| 9 | 1.80E−6 | 68 |
| 10 | 4.35E−9 | 54 |
| 11 | 3.25E−9 | 68 |
| 12 | 5.80E−10 | 89 |
| 13 | 3.20E−10 | 93 |
| 14 | >50000 | 0 |
| 15 | 1.60E−9 | 95 |
| 16 | >5.00E−5 | 0 |
| 17 | >5.00E−5 | 0 |
| 18 | >3.30E−5 | 0 |
| 19 | >5.00E−5 | 0 |
| 20 | 8.55E−9 | 113 |
| 21 | >5.00E−5 | 0 |
| 22 | >5.00E−5 | 0 |
| 23 | 9.35e−6 | 97 |
| 24 | 5.75E−6 | 94 |
| 25 | 4.00E−9 | 77 |
| 26 | 2.20E−10 | 88 |
| 27 | 2.85E−10 | 115 |

### Biochemical Assay for Determining Inverse Agonist Activity of Compounds

A biochemical interaction assay measuring the ligand-dependent changes in interactions between the PPARG-LBD and a fluorescent peptide from the corepressor, NCOR2 (Smrt ID2, ThermoFisher), was performed to evaluate inverse agonist activity.

### Test Procedure:

This assay was performed according to the manufacturers protocol (LanthaScreen TR-FRET PPAR gamma Corepressor Assay, ThermoFisher). PPARG-LBD, corepressor peptide NCOR2, LanthaScreen™ Tb-anti-GST antibody and compounds in 10 concentrations ranging from 50 μM to 0.03 nM were incubated in 15 μl per well of a 384 small volume black non-binding microtiter plate (Greiner) in Tris buffer (50 mM Tris, 50 mM NaCl, 20 mM MgCl2, 1 mM EDTA, 0.01% Casein (Sigma), 0,0003% Tween 20 (Sigma), 1 mM OTT (Sigma), pH 7.5) for 2 hours at room temperature. After incubation the FRET signal was measured using an PHERAstar Plus Plate Reader (BMG). Every sample was tested in duplicate and mean values were used to calculate the EC50 values and efficacies of the compounds. A model of nonlinear regression curve fit was used for calculation.

TABLE 2

| Example No. | IC50 - [mol/l] | Efficacy - [%] |
|---|---|---|
| 1 | 1.76E−9 | 345 |
| 2 | 5.20E−9 | 99 |
| 3 | 5.81E−9 | 619 |
| 4 | 7.15E−9 | 509 |
| 5 | 3.93E−9 | 235 |
| 6 | 2.18E−9 | 534 |
| 7 | 6.48E−10 | 437 |
| 8 | 2.00E−9 | 580 |
| 9 | 3.95E−9 | 524 |
| 10 | 3.70E−9 | 95 |
| 11 | 1.90E−8 | 424 |
| 12 | 2.57E−9 | 647 |
| 13 | 4.36E−9 | 562 |
| 14 | >5.00E−5 | 0 |
| 15 | 1.01E−8 | 288 |
| 16 | 5.35E−8 | 331 |
| 17 | >5.00E−5 | 0 |
| 18 | >5.00E−5 | 0 |
| 19 | >5.00E−5 | 0 |
| 20 | 8.65E−8 | 335 |
| 21 | 8.05E−6 | 554 |
| 22 | >5.00E−5 | 0 |
| 23 | 7.95E−6 | 86 |
| 24 | >5.00E−5 | 0 |
| 25 | 3.30E−9 | 638 |
| 26 | 9.56E−9 | 621 |
| 27 | 2.80E−8 | 690 |

### Proliferation UM-UC-9 H2B-GFP Assay

Instrument: Incucyte® (Sartorius).

### Description:

Incucyte® Proliferation Assay measures cell proliferation based on nuclei count using live-cell time-lapse imaging. UMUC9 cells (Sigma—ECaCC 08090505) transduced with H2B-eGFP were seeded in 384-well plates (Corning #353962), at a density of 500 cells/well, in 40 μl/well MEM-alpha phenol-red free medium (Gibco Cat #41061-029) supplemented with 10% fetal calf serum. Next day cells were treated with compounds at indicated concentration with max 0.25% DMSO using an HP dispenser instrument. Measurements were performed on the day of treatment (Day 1 reading) and after 7 days of treatment (Day 8 reading). Green fluorescence in cell nuclei is measured using the instrument (Image Channels: Phase, Green; Acquisition time: 300 ms; Magnification: 4×).

### Analysis:

Selected metrics for analysis: Phase Object Confluence (Percent), Nuclei Count (1/Well) and Total Nuclei Area (μm²/Well). Day 1 measurements were subtracted from each Day 8 measurement and expressed as percentage of the vehicle (DMSO) control. Percentage proliferation values were then plotted against compound concentration using GraphPad PRISM software. The Sigmoidal 4PL X is log(concentration) model of linear regression was used. Relative IC50 values and % efficacy (span) is reported below.

TABLE 3

| Example | UM-UC-9 GFP proliferation | UM-UC-9 GFP proliferation |
|---|---|---|
| No. | IC50 - [mol/l] | Efficacy - [%] |
| 1 | >1.00E−5 | 0 |
| 2 | 6.64E−6 | |
| 3 | 3.39E−9 | 79 |
| 4 | n.d. | n.d. |
| 5 | n.d. | n.d. |
| 6 | 3.14e−7 | 67 |
| 7 | 1.92E−9 | 34 |
| 8 | 3.12E−9 | 86 |
| 9 | 3.71E−6 | 105 |
| 10 | 9.13E−7 | n.d. |
| 11 | 9.19E−9 | 36 |
| 12 | 5.18E−8 | 55 |
| 13 | 2.75E−9 | 36 |
| 14 | n.d. | n.d. |
| 15 | 8.80E−9 | 43 |
| 16 | >1.00E−5 | 0 |
| 17 | n.d. | n.d. |
| 18 | n.d | n.d. |
| 19 | n.d. | n.d. |
| 20 | 3.58E−7 | 84 |
| 21 | n.d. | n.d. |
| 22 | 1.22E−6 | 22 |
| 23 | n.d. | n.d. |
| 24 | n.d. | n.d. |
| 25 | 3.30E−7 | 91 |
| 26 | 2.54E−9 | 72 |
| 27 | 5.25E−9 | 78 |

### UM-UC-9-Proliferation Assay (Basis for FIG. 1):

UM-UC-9 cells were purchased from European Collection of Authenticated Cell Cultures (EcACC) and grown in MEM alpha media (Gibco) containing 10% heat-inactivated fetal bovine serum (Sigma). The cells were stably transduced with a lentiviral expression vector encoding the TagGFP-Histone-2B protein (pTagGFP2-H2B, Evrogen). Cells were plated at 500 cells per ACTIVE 68996420201 well in a 384-well view-plate and allowed to attach at 37 C for 2 hours. Plates were dosed with the compound of example 3 at indicated concentration in single replicate in 14 point dose-response curve. 5 days after addition of compound, plates were imaged using an IncuCyte S3 Live Cell Imager and nuclei were counted. Cell counts were normalized to vehicle control and data reported as percent of vehicle control. The compound of example 3 reported was also evaluated in an independent experiment read out at day 7 with similar result.
Open box □: Rosiglitazone
Closed triangle ▴: SR10221
Open Triangle Δ: T007907
Open circle ∘: compound of example 26
Closed circle •: compound of example 3

The order of endpoints of the curves is from top to bottom: open box curve, open triangle curve, closed triangle curve, and closed circle curve almost identical with open circle curve.

### Colony Formation Assay and Crystal Violet Staining (Basis for FIG. 2)

Cells were plated in triplicate in 12-well plates in 1 mL of media per well and allowed to adhere prior to treatment with vehicle or compound. All cell lines were grown in MEMα containing 10% FBS, with the exception of PaCaDD-188 and PaCaDD-161, which were grown in Dresden media (2:1 ratio DMEM (Gibco) with 20% FBS and Keratinocyte SFM (serum-free medium) (Gibco). Plating density was determined prior in order to achieve approximately 80% confluency in 7-14 days with vehicle treatment. For the experimental plates, cells were treated with DMSO vehicle, neutral antagonist GW9662 (100 nM), T0070907 (100 nM), or the compound of example 3 (100 nM) using the HP-D300e Digital Dispenser (Tecan). Every 3-4 days, media and compounds were replenished. When vehicle-treated cells reached approximately 80% confluency, cells were stained with crystal violet. Briefly, media was removed and cells were washed with PBS. Cells were fixed using 4% formaldehyde in PBS for 30 min at room temperature. The formaldehyde solution was removed and the cells were stained with crystal violet staining solution (0.1% crystal violet in solution 10% ethanol and 90% deionized water) for another 30 min, after which the stain was removed and washed out with deionized water. The plates were allowed to dry inverted overnight and imaged the following day using an Epson Perfection 600 scanner.

1. A compound of formula (I) 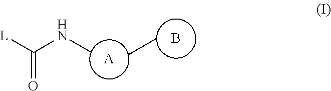
wherein,
L is selected from 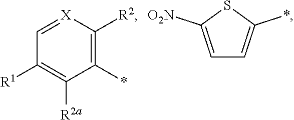
and an ethenylene group
R¹ is selected from a hydrogen atom, a NO₂ group, a cyano group, a —C═(O)NH—(C₁-C₃-alkyl) group, a —C═(O)NH—(C₂-C₃-alkenyl) group and C₁-C₃-haloalkyl group;
R² is selected from a hydrogen atom and a halogen atom;
R^{2a} is selected from a hydrogen atom or a halogen atom;
X is selected from a CR³ group and a nitrogen atom
R³ is a hydrogen atom;
Ring A is selected from the groups (a) to (h) 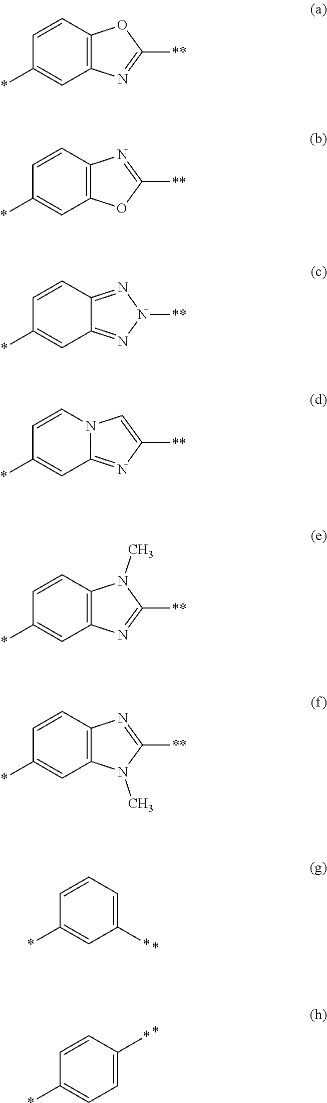
whereby * marks the position where ring A is bound to the nitrogen atom of the amide and ** marks the position where ring A is bound to ring B;
B is selected from the groups (i) to (l) 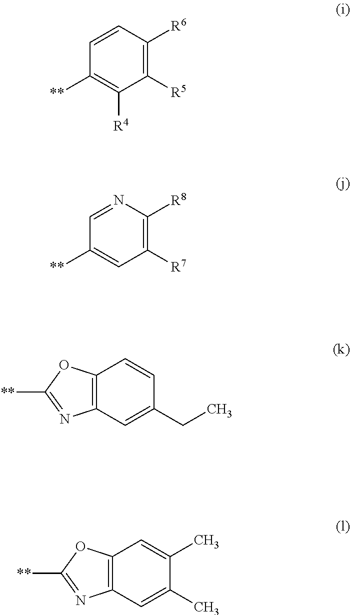
whereby ** is the point of attachment to the A ring,
R⁴ is selected from the group consisting of a hydrogen atom, a halogen atom and a C₁-C₃-alkyl group;
R⁵ is selected from the group consisting of a hydrogen atom, a halogen atom, a C₁-C₃-alkyl group;
R⁶ is selected from the group consisting of a hydrogen atom, a C₁-C₃-alkyl group, a C₁-C₃-alkoxy group;
R⁷ is a hydrogen atom or a C₁-C₃-alkyl group;
R⁸ is a hydrogen atom or a C₁-C₃-alkyl group;
with the proviso, that those compounds are excluded for which
R¹ is a nitro group,
R² is a chlorine atom,
R^{2a} is a hydrogen atom,
X is a CH group,
ring A is a benzoxazole (a),
R⁴ is selected from the group consisting of a hydrogen atom, a halogen atom and a C₁-C₃-alkyl group,
R⁵ is selected from the group consisting of a hydrogen atom, a halogen atom, a C₁-C₃-alkyl group and
R⁶ is selected from the group consisting of a hydrogen atom, a C₁-C₃-alkyl group, a C₁-C₃-alkoxy group,
or a stereoisomer, a tautomer, an N-oxide, a hydrate, a solvate, or a salt thereof, or a mixture of same.

2. The compound of claim 1,
wherein, independently from each occurrence
L is selected from 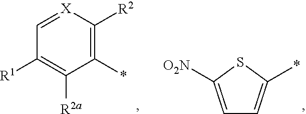
R¹ is selected from a hydrogen atom, a NO₂ group, a cyano group, a —C═(O)NH—CH₃ group, a —C(═O)NH—CH═CH₂ group, and a CF₃ group;
R² is selected from a hydrogen atom, a fluorine atom, a chlorine atom, a bromine atom and an iodine atom;
R^{2a} is a hydrogen atom;
X is a CR³ group and a nitrogen atom;
R³ is a hydrogen atom;
A is selected from the groups (a) to (h) 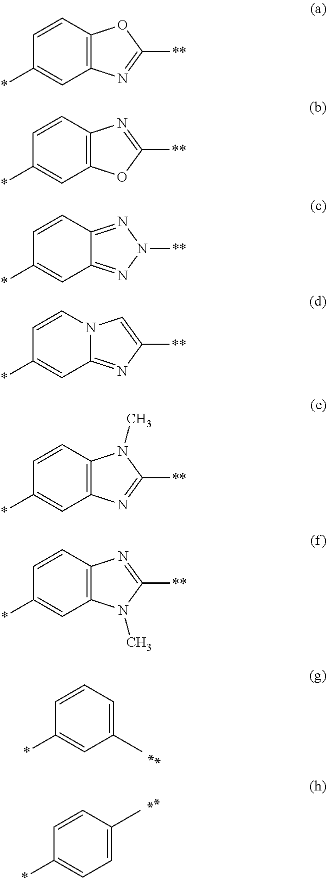
whereby * marks the position where ring A is bound to the nitrogen atom of the amide and ** marks the position where ring A is bound to ring B;
B is selected from the groups (i) to (l) 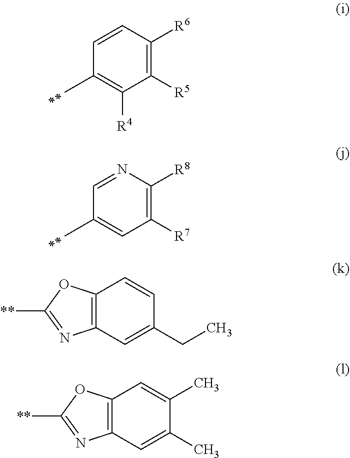
whereby ** is the point of attachment to the A ring;
R⁴ is selected from the group consisting of a hydrogen atom, a bromine atom and a methyl group;
R⁵ is selected from the group consisting of a hydrogen atom, a fluorine atom, a chlorine atom, and a methyl group;
R⁶ is selected from the group consisting of a hydrogen atom, a methyl group, an ethyl group, and a methoxy group;
R⁷ is a hydrogen atom or a methyl group;
R⁸ is a hydrogen atom or an ethyl group;
with the proviso, that those compounds are excluded for which
R¹ is a nitro group,
R² is a chlorine atom,
R^{2a} is a hydrogen atom,
X is a CH group,
ring A is a benzoxazole (a),
R⁴ is selected from the group consisting of a hydrogen atom, a halogen atom and a methyl group,
R⁵ is selected from the group consisting of a hydrogen atom, a fluorine atom, a chlorine atom, a methyl group and
R⁶ is selected from the group consisting of a hydrogen atom, a methyl group, a methoxy group,
or a stereoisomer, a tautomer, an N-oxide, a hydrate, a solvate, or a salt thereof, or a mixture of same.

3. The compound of claim 1,
wherein, independently from each occurrence
L is selected from 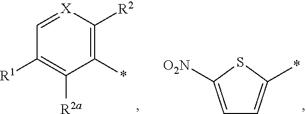
R¹ is selected from a hydrogen atom, a NO₂ group, a cyano group, a —C═(O)NH—CH₃ group, a —C(═O)NH—CH═CH₂ group, and a CF₃ group;
R² is selected from a hydrogen atom, a fluorine atom, a chlorine atom, a bromine atom and an iodine atom;
R^{2a} is a hydrogen atom;
X is a CR³ group and a nitrogen atom;
R³ is a hydrogen atom;
A is selected from the groups (a) to (h) 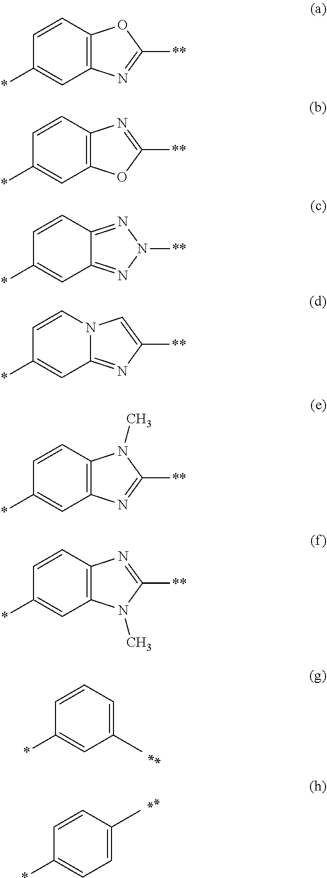
whereby * marks the position where ring A is bound to the nitrogen atom of the amide and ** marks the position where ring A is bound to ring B;
B is selected from the groups (i) to (j) 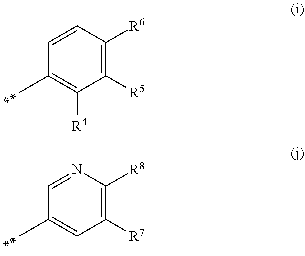
whereby ** is the point of attachment to the A ring;
R⁴ is selected from the group consisting of a hydrogen atom, a bromine atom and a methyl group;
R⁵ is selected from the group consisting of a hydrogen atom, a fluorine atom, a chlorine atom, and a methyl group;
R⁶ is selected from the group consisting of a hydrogen atom, a methyl group, an ethyl group, and a methoxy group;
R⁷ is a hydrogen atom or a methyl group;
R⁸ is a hydrogen atom or an ethyl group;
with the proviso, that those compounds are excluded for which
R¹ is a nitro group,
R² is a chlorine atom,
R^{2a} is a hydrogen atom,
X is a CH group,
ring A is a benzoxazole (a),
R⁴ is selected from the group consisting of a hydrogen atom, a halogen atom and a methyl group,
R⁵ is selected from the group consisting of a hydrogen atom, a fluorine atom, a chlorine atom, a methyl group and
R⁶ is selected from the group consisting of a hydrogen atom, a methyl group, a methoxy group,
or a stereoisomer, a tautomer, an N-oxide, a hydrate, a solvate, or a salt thereof, or a mixture of same.

4. The compound according to claim 1 selected from the group consisting of
2-Chloro-N-[2-(3-methylphenyl)-1,3-benzoxazol-5-yl]-5-nitrobenzamide
2-Chloro-N-[2-(2-methylphenyl)-1,3-benzoxazol-5-yl]-5-nitrobenzamide
2-Chloro-N-[2-(4-ethylphenyl)-1,3-benzoxazol-5-yl]-5-nitrobenzamide
2-Chloro-N-[2-(3-chlorophenyl)-1,3-benzoxazol-5-yl]-5-nitrobenzamide
2-Chloro-N-[2-(3-fluoro-4-methoxyphenyl)-1,3-benzoxazol-5-yl]-5-nitrobenzamide
2-Chloro-N-[2-(5-methylpyridin-3-yl)-1, 3-benzoxazol-5-yl]-5-nitrobenzamide
2-Chloro-N-[2-(6-ethylpyridin-3-yl)-1, 3-benzoxazol-5-yl]-5-nitrobenzamide
2-Chloro-N-[2-(4-methylphenyl)-1,3-benzoxazol-6-yl]-5-nitrobenzamide
2-Chloro-N-[2-(4-ethylphenyl)-1-methyl-1H-benzimidazol-5-yl]-5-nitrobenzamide
2-Chloro-N-[2-(4-ethylphenyl)-1-methyl-1H-benzimidazol-6-yl]-5-nitrobenzamide
2-Chloro-5-nitro-N-(2-phenylimidazo[1,2-a]pyridin-6-yl)benzamide
2-Chloro-N-[2-(4-methylphenyl)-2H-benzotriazol-5-yl]-5-nitrobenzamide
2-Chloro-N-[2-(4-ethylphenyl)-1,3-benzoxazol-5-yl]-5-nitrobenzene-1-sulfonamide
2-chloro-N-[4-(5, 6-dimethyl-1,3-benzoxazol-2-yl)phenyl]-5-nitrobenzamide
2-Chloro-N-[2-(4-ethylphenyl)-1,3-benzoxazol-5-yl]-5-(trifluoromethyl)benzamide
4-Chloro-N³-[2-(4-ethylphenyl)-1,3-benzoxazol-5-yl]-N¹-methylbenzene-1,3-dicarboxamide
2-Chloro-N-[2-(4-ethylphenyl)-1,3-benzoxazol-5-yl]pyridine-3-carboxamide
2-chloro-5-cyano-N-[2-(4-ethylphenyl)-1,3-benzoxazol-5-yl]benzamide
N-[2-(4-Ethylphenyl)-1,3-benzoxazol-5-yl]-3-nitrobenzamide
2-Chloro-N-[2-(3-methylphenyl)-1,3-benzoxazol-5-yl]-3-nitrobenzamide
N-[2-(4-Ethylphenyl)-1,3-benzoxazol-5-yl]-5-nitrothiophene-2-carboxamide
N-[2-(4-Ethylphenyl)-1,3-benzoxazol-5-yl]prop-2-enamide
N-[2-(4-Ethylphenyl)-1,3-benzoxazol-5-yl]-2-fluoro-5-nitrobenzamide
2-Bromo-N-[2-(4-ethylphenyl)-1,3-benzoxazol-5-yl]-5-nitrobenzamide
N-[2-(4-ethylphenyl)-1,3-benzoxazol-5-yl]-2-iodo-5-nitrobenzamide
or a stereoisomer, a tautomer, an N-oxide, a hydrate, a solvate, or a salt thereof, or a mixture of same.

5. A method of preparing a compound of general formula (I) according to claim 1, said method comprising
reacting a compound of formula (IIa) 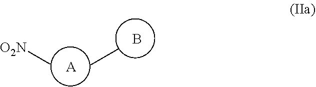
wherein
Ring A is selected from the groups (a) to (h) 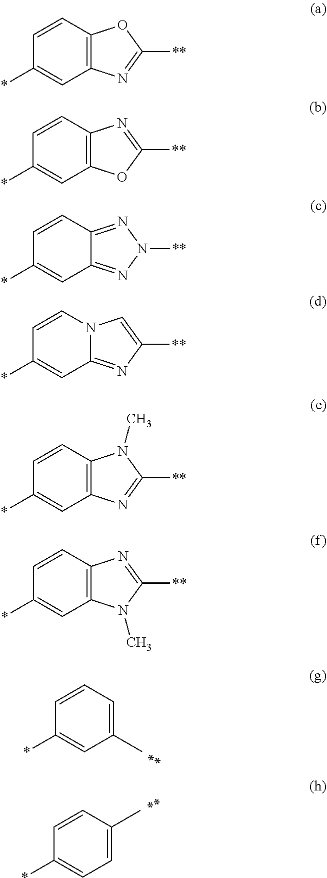
whereby * marks the position where ring A is bound to the nitrogen atom of the amide and ** marks the position where ring A is bound to ring B;
B is selected from the groups 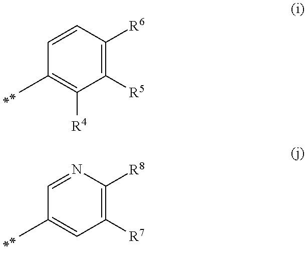
whereby ** is the point of attachment to the A ring,
by reducing the nitro group of the compound of formula (IIa) with a reducing agent in an organic alcohol solution or transforming the halogen atom into an amine obtaining a compound of formula (III) 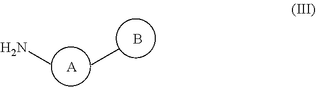
and subsequently forming an amide by reacting the compound of formula (III) with an organic acid of formula (IV) or (V), optionally having protected reactive substituents, 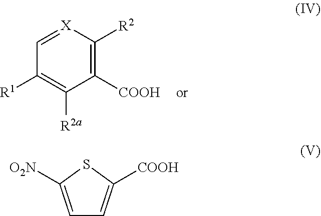
and optionally activating the acid with an activating agent under addition of an organic base in order to obtain a compound of formula

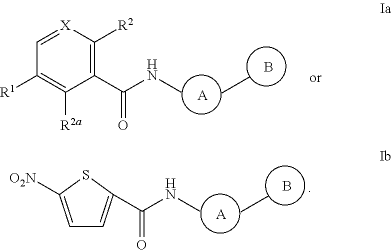

6. A method of preparing a compound of general formula (I) according to claim 1, said method comprising
transforming a compound of formula (IIb) 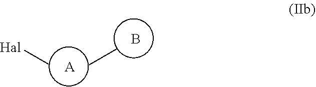
wherein Hal is a chlorine atom or a bromine atom and ring A and Ring B are as defined above,
into the tert.-butylcarbamate under basic conditions by reacting with sodium- or potassium-ter.butanolate followed by a palladium catalysed reduction in order to obtain the amine of formula (III) 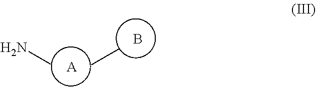
and subsequently forming an amide by reacting the compound of formula (III) with an organic acid of formula (IV) or (V), optionally having protected reactive substituents, 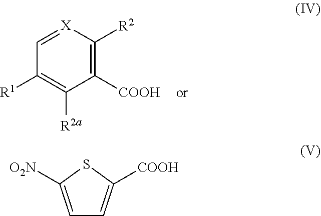
and optionally activating the acid with an activating agent under addition of an organic base in order to obtain a compound of formula (Ia) or formula (Ib)

7. A pharmaceutical composition comprising a compound of general formula (I) according to claim 1 and one or more pharmaceutically acceptable excipients.

8. A pharmaceutical combination comprising:
one or more first active ingredients, in particular compounds of general formula (I) according to claim 1, and
one or more further active ingredients.

9. A method for the preparation of a medicament for the treatment or prophylaxis of a disease comprising combining the compound of general formula (I) with one or more pharmaceutically acceptable excipients.

10. A method for the treatment or prophylaxis of a disease in a subject in need thereof comprising administering the compound of general formula (I) according to claim 1 to the subject.

11. The method according to claim 10, wherein the disease is a hyperproliferative disease.

12. The method according to claim 11, wherein the hyperproliferative disease is a cancer disease.

13. The method according to claim 12, wherein the cancer disease is selected from a cancer appearing in an organ selected from the anus, the brain, the breast, the bones, the central and peripheral nervous system, the colon, the eye, the kidney, the endocrine glands, the endometrium, the esophagus, the gastrointestinal tract the germ cells, the head and the neck, the kidney, the liver, the larynx and hypopharynx, the lung, the mesothelioma, the pancreas, the prostate, the rectum, the reproductive organs, the respiratory tract, the small intestine, the skin, the soft tissue, the stomach, the testis, the thyroid gland, the parathyroid gland, ureter, the urogenital tract, vagina and vulva and the connective tissue and metastases of these tumors.

14. The method according to claim 12, wherein the cancer disease is selected from breast cancer, esophageal cancer, pancreatic cancer, colorectal cancer, hepatocellular cancer, bladder cancer.

15. The method according to claim 12, wherein the cancer disease is selected from bladder cancer, pancreatic cancer and colon cancer.

16. A method of treating cancer in a subject, the method comprising administering to the subject the compound of claim 1, thereby treating the cancer.
